SEQUENCE LISTING (141 sequences)

SEQ ID NO: 1 - protein, 163 aa, Chlamydomonas reinhardtii
MNTKYNKEFLLYLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 2 - DNA, 22 bp, Artificial
  C1221 target
caaaacgtcgtacgacgttttg

SEQ ID NO: 3 - protein, 167 aa, Artificial
  I-CreI  N75
MANTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSPAAD

SEQ ID NO: 4 - DNA, 24 bp, Artificial
  DMD21 target
gaaacctcaagtaccaaatgtaaa

SEQ ID NO: 5 - DNA, 24 bp, Artificial
  10AAC_P target
tcaacacgtcgtacgacgtgttga

SEQ ID NO: 6 - DNA, 24 bp, Artificial
  5CAA_P target
tcaaaaccaagtacttggttttga

SEQ ID NO: 7 - DNA, 24 bp, Artificial
  10TAC_P target
tctacacgtcgtacgacgtgtaga

SEQ ID NO: 8 - DNA, 24 bp, Artificial
  5TTG_P target
tcaaaacttggtaccaagttttga

SEQ ID NO: 9 - DNA, 24 bp, Artificial
  DMD21.3 target
gaaacctcaagtacttgaggtttc

SEQ ID NO: 10 - DNA, 24 bp, Artificial
  DMD21.4 target
tttacatttggtaccaaatgtaaa

SEQ ID NO: 11 - DNA, 24 bp, Artificial
  DMD24 target
tttacctattttaagtcagataca

SEQ ID NO: 12 - DNA, 24 bp, Artificial
  10TAC_P target
tctacacgtcgtacgacgtgtaga

SEQ ID NO: 13 - DNA, 24 bp, Artificial
  5ATT_P target
tcaaaacattgtacaatgttttga

SEQ ID NO: 14 - DNA, 24 bp, Artificial
  10TAT_P target
tctatacgtcgtacgacgtataga

SEQ ID NO: 15 - DNA, 24 bp, Artificial
  5GAC_P target
tcaaaacgacgtacgtcgttttga

SEQ ID NO: 16 - DNA, 24 bp, Artificial
  DMD24.2 target
tttacctattgtacgtcagataca

SEQ ID NO: 17 - DNA, 24 bp, Artificial
  DMD24.3 target
tttacctattgtacaataggtaaa

SEQ ID NO: 18 - DNA, 24 bp, Artificial
  DMD24.4 target
tgtatctgacgtacgtcagataca

SEQ ID NO: 19 - DNA, 24 bp, Artificial
  DMD24.5 target
tttacctattttaaaataggtaaa

SEQ ID NO: 20 - DNA, 24 bp, Artificial
  DMD24.6 target
tgtatctgacttaagtcagataca

SEQ ID NO: 21 - DNA, 24 bp, Artificial
  DMD31 target
aatgtctgatgttcaatgtgttga

SEQ ID NO: 22 - DNA, 24 bp, Artificial
  10TGT_P target
tctgtacgtcgtacgacgtacaga

SEQ ID NO: 23 - DNA, 24 bp, Artificial
  5GAT_P target
tcaaaacgatgtacatcgttttga

SEQ ID NO: 24 - DNA, 24 bp, Artificial
  10AAC_P target
tcaacacgtcgtacgacgtgttga

SEQ ID NO: 25 - DNA, 24 bp, Artificial
  5ATT_P target
tcaaaacattgtacaatgttttga

SEQ ID NO: 26 - DNA, 24 bp, Artificial
  DMD31.2 target
aatgtctgatgtacaatgtgttga

SEQ ID NO: 27 - DNA, 24 bp, Artificial
  DMD31.3 target
aatgtctgatgtacatcagacatt

SEQ ID NO: 28 - DNA, 24 bp, Artificial
  DMD31.4 target
tcaacacattgtacaatgtgttga

SEQ ID NO: 29 - DNA, 24 bp, Artificial
  DMD31.5 target
aatgtctgatgttcatcagacatt

SEQ ID NO: 30 - DNA, 24 bp, Artificial
  DMD31.6 target
tcaacacattgttcaatgtgttga

SEQ ID NO: 31 - DNA, 24 bp, Artificial
  DMD33 target
aaatcctgccttaaagtatctcat

SEQ ID NO: 32 - DNA, 24 bp, Artificial
  10ATC_P target
tcatcacgtcgtacgacgtgatga

SEQ ID NO: 33 - DNA, 24 bp, Artificial
  5GCC_P target
tcaaaacgccgtacggcgttttga

SEQ ID NO: 34 - DNA, 24 bp, Artificial
  10GAG_P target
tcgagacgtcgtacgacgtctcga

SEQ ID NO: 35 - DNA, 24 bp, Artificial
  5ACT_P target
tcaaaacactgtacagtgttttga

SEQ ID NO: 36 - DNA, 24 bp, Artificial
  DMD33.2 target
aaatcctgccgtacagtatctcat

SEQ ID NO: 37 - DNA, 24 bp, Artificial
  DMD33.3 target
aaatcctgccgtacggcaggattt

SEQ ID NO: 38 - DNA, 24 bp, Artificial
  DMD33.4 target
atgagatactgtacagtatctcat

SEQ ID NO: 39 - DNA, 24 bp, Artificial
  DMD33.5 target
aaatcctgccttaaggcaggattt

SEQ ID NO: 40 - DNA, 24 bp, Artificial
  DMD33.6 target
atgagatactttaaagtatctcat

SEQ ID NO: 41 - DNA, 24 bp, Artificial
  DMD35 target
tctttatgttttaaagtatattcc

SEQ ID NO: 42 - DNA, 24 bp, Artificial
  10TTT_P target
tctttacgtcgtacgacgtaaaga

SEQ ID NO: 43 - DNA, 24 bp, Artificial
  5GTT_P target
tcaaaacgttgtacaacgttttga

SEQ ID NO: 44 - DNA, 24 bp, Artificial
  10AAT_P target
tcaatacgtcgtacgacgtattga

SEQ ID NO: 45 - DNA, 24 bp, Artificial
  5ACT_P target
tcaaaacactgtacagtgttttga

SEQ ID NO: 46 - DNA, 24 bp, Artificial
  DMD35.2 target
tctttatgttgtacagtatattcc

SEQ ID NO: 47 - DNA, 24 bp, Artificial
  DMD35.3 target
tctttatgttgtacaacataaaga

SEQ ID NO: 48 - DNA, 24 bp, Artificial
  DMD35.4 target
ggaatatactgtacagtatattcc

SEQ ID NO: 49 - DNA, 24 bp, Artificial
  DMD35.5 target
tctttatgttttaaaacataaaga

SEQ ID NO: 50 - DNA, 24 bp, Artificial
  DMD35.6 target
ggaatatactttaaagtatattcc

SEQ ID NO: 51 - DNA, 24 bp, Artificial
  DMD37.1 target
gaatcctgttgttcatcatcctag

SEQ ID NO: 52 - DNA, 24 bp, Artificial
  10ATC_P target
tcatcacgtcgtacgacgtgatga

SEQ ID NO: 53 - DNA, 24 bp, Artificial
  5GTT_P target
tcaaaacgttgtacaacgttttga

SEQ ID NO: 54 - DNA, 24 bp, Artificial
  10AGG_P target
tcaggacgtcgtacgacgtcctga

SEQ ID NO: 55 - DNA, 24 bp, Artificial
  5GAT_P target
tcaaaacgatgtacatcgttttga

SEQ ID NO: 56 - DNA, 24 bp, Artificial
  DMD37.2 target
gaatcctgttgtacatcatcctag

SEQ ID NO: 57 - DNA, 24 bp, Artificial
  DMD37.3 target
gaatcctgttgtacaacaggattc

SEQ ID NO: 58 - DNA, 24 bp, Artificial
  DMD37.4 target
ctaggatgatgtacatcatcctag

SEQ ID NO: 59 - DNA, 24 bp, Artificial
  DMD37.5 target
gaatcctgttgttcaacaggattc

SEQ ID NO: 60 - DNA, 24 bp, Artificial
  DMD37.6 target
ctaggatgatgttcatcatcctag

SEQ ID NO: 61 - protein, 32 aa, Artificial
  RM2 peptidic linker
AAGGSDKYNQALSKYNQALSKYNQALSGGGGS

SEQ ID NO: 62 - protein, 354 aa, artificial sequence
  SCOH-DMD21-pCLS2872
MANTKYNEEFLLYLAGFVDGDGSIIAQIKPRQSYKFKHQLELTFTVGQKTQRRWFLDKLVDEIGVGYVTDSGSMSAYRLSKIKPLHNFLTQLQPFLELKQKQANLVLKIIEQLPSAKESPDKFLEVCTWVDQIAALNDSKTRKTTSETVRAVLDNLSEKKKSSPAAGGSDKYNQALSKYNQALSKYNQALSGGGGSNKKFLLYLAGFVDSDGSIIAQIRPNQSAKFKHYLQLTFQVTQKTQRRWFLDKLVDRIGVGYVRDSGSVSDYKLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 63 - protein, 354 aa, artificial sequence
  SCOH-DMD21-pCLS2873
MANTKYNEEFLLYLAGFVDGDGSIIAQIKPRQSYKFKHQLELTFTVGQKTQRRWFLDKLVDEIGVGYVTDSGSMSAYRLSKIKPLHNFLTQLQPFLELKQKQANLVLKIIEQLPSAKESPDKFLEVCTWVDQVAALNDSKTRKTTSETVRAVLDNLSEKKKSSPAAGGSDKYNQALSKYNQALSKYNQALSGGGGSNKKFLLYLAGFVDSDGSIIAQIRPNQSAKFKHYLQLTFQVTQKTQRRWFLDKLVDRIGVGYVRDSGSVSDYKLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 64 - protein, 354 aa, artificial sequence
  SCOH-DMD21-pCLS2874
MANTKYNEEFLLYLAGFVDGDGSIIAQIKPRQSYKFKHQLELTFTVGQKTQRRWFLDKLVDEIGVGYVTDSGSMSAYRLSKIKPLHNFLTQLQPFLELKQKQANLVLKIIEQLPSAKESPDKFLEVCTWVDQVAALNDSKTRKTTSETVRAVLDNLSEKKKSSPAAGGSDKYNQALSKYNQALSKYNQALSGGGGSNKKFLLYLAGFVDSDGSIIAQIRPNQSAKFKHYLQLTFQVTQKTQRRWFLDKLVDRIGVGYVRDSGSVSDYKLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 65 - protein, 354 aa, artificial sequence
  SCOH-DMD21-pCLS2875
MANTKYNEEFLLYLAGFVDGDGSIIAQIKPRQSYKFKHQLELTFTVGQKTQRRWFLDKLVDEIGVGYVTDSGSMSAYRLSKIKPLHNFLTQLQPFLELKQKQANLVLKIIEQLPSAKESPDKFLEVCTWVDQIAALNDSKTRKTTSETVRAVLDNLSEKKKSSPAAGGSDKYNQALSKYNQALSKYNQALSGGGGSNKKFLLYLAGFVDSDGSIIAQIRPNQSAKFKHYLQLTFQVTQKTQRRWFLDKLVDRIGVGYVRDSGSVSDYKLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 66 - protein, 354 aa, artificial sequence
  SCOH-DMD21-pCLS3385
MANTKYNEEFLLYLAGFVDGDGSIIAQIKPRQSYKFKHQLELTFTVGQKTQRRWFLDKLVDEIGVGYVTDSGSMSAYRLSKIKPLHNFLTQLQPFLELKQKQANLALKIIEQLPSAKESPDKFLEVCTWVDQVAALNDSKTRKTTSETVRAVLDNLSEKKKSSPAAGGSDKYNQALSKYNQALSKYNQALSGGGGSNKKFLLYLAGFVDSDGSIIAQIRPNQSAKFKHYLQLTFQVTQKTQRRWFLDKLVDRIGVGYVRDSGSVSDYKLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 67 - protein, 354 aa, artificial sequence
  SCOH-DMD21-pCLS3387
MANTKYNEEFLLYLAGFVDGDGSIIAQIKPRQSYKFKHQLELTFTVGQKTQRRWFLDKLVDEIGVGYVTDSGSMSAYRLSKIKPLHNFLTQLQPFLELKQKQANLVLKIIEQLPSAKESPDKFLEVCTWVDQVAALNDSKTRKTTSETVRAVLDNLSEKKKSSPAAGGSDKYNQALSKYNQALSKYNQALSGGGGSNKKFLLYLAGFVDSDGSIIAQIRPNQSAKFKHYLQLTFQVTQKTQRRWFLDKLVDRIGVGHVRDSGSVSDYKLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 68 - protein, 354 aa, artificial sequence
  SCOH-DMD21-pCLS3388
MANTKYNEEFLLYLAGFVDGDGSIIAQIKPRQSYKFKHQLELTFTVGQKTQRRWFLDKLVDEIGVGYVTDSGSMSAYRLSKIKPLHNFLTQLQPFLELKQKQANLALKIIEQLPSAKESPDKFLEVCTWVDQVAALNDSKTRKTTSETVRAVLDNLSEKKKSSPAAGGSDKYNQALSKYNQALSKYNQALSGGGGSNKKFLLYLAGFVDSDGSIIAQIRPNQSAKFKHYLQLTFQVTQKTQRRWFLDKLVDRIGVGYVRDSGSVSDYKLSKIKPLHNFLTQLQPFLKLKQKQANLALKIIEQLPSAKESPDKFLEVCTWVDQVAALNDSKTRKTTSETVRAVLDSLSEKKKSSP

SEQ ID NO: 69 - protein, 354 aa, artificial sequence
  SCOH-DMD24-pCLS3397
MANTKYNEEFLLYLAGFVDGDGSIVAQIKPNQGCKFKHQLSLTFQVTQKTQRRWFLDKLVDEIGVGYVADSGSVSNYRLGKIKPLHNFLTQLQPFLELKQKQANLVLKIIEQLPSAKESPDKFLEVCTWVDQVAALNDSKTRKTTSETVRAVLGSLSEKKKSSPAAGGSDKYNQALSKYNQALSKYNQALSGGGGSNKKFLLYLAGFVDSDGSIIAQIKPNQHCKFKHQLALTFYVTQKTQRRWFLDKLVDRIGVGYVYDSGSVSRYVLSEIKPLHNFLTQLQPFLKLKQKQANLALKIIEQLPSAKESPDKFLEVCTWVDQVAALNDSKTRKTTSETVRAVLDSLSEKKKSSP

SEQ ID NO: 70 - protein, 354 aa, artificial sequence
  SCOH-DMD24-pCLS3399
MANTKYNEEFLLYLAGFVDGDGSIVAQIKPNQGCKFKHQLSLTFQVTQKTQRRWFLDKLVDEIGVGYVADSGSVSNYRLGKIKPLHNFLTQLQPFLELKQKQANLALKIIEQLPSAKESPDKFLEVCTWVDQVAALNDSKTRKTTSETVRAVLGSLSEKKKSSPAAGGSDKYNQALSKYNQALSKYNQALSGGGGSNKKFLLYLAGFVDSDGSIIAQIKPNQHCKFKHQLALTFYVTQKTQRRWFLDKLVDRIGVGYVYDSGSVSRYVLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 71 - protein, 354 aa, artificial sequence
  SCOH-DMD24-pCLS3400
MANTKYNEEFLLYLAGFVDGDGSIVAQIKPNQGCKFKHQLCLTFQVTQKTQRRWFLDKLVDEIGVGYVADSGSVSNYRLSEIKPLHNFLTQLQPFLELKQKQANLVLKIIEQLPSAKESPDKFLEVCTWVDQIAALNDSKTRKTTSETVRAVLDSLSEKKKSSPAAGGSDKYNQALSKYNQALSKYNQALSGGGGSNKKFLLYLAGFVDSDGSIIAQIKPNQHCKFKHQLALTFYVTQKTQRRWFLDKLVDRIGVGYVYDSGSVSRYVLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 72 - protein, 354 aa, artificial sequence
  SCOH-DMD24-pCLS3401
MANTKYNEEFLLYLAGFVDGDGSIVAQIKPNQGCKFKHQLCLTFQVTQKTQRRWFLDKLVDEIGVGYVADSGSVSNYRLSEIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQHCKFKHQLALTFYVTQKTQRRWFLDKLVDRIGVGYVYDSGSVSRYVLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 73 - protein, 354 aa, artificial sequence
  SCOH-DMD24-pCLS3402
MANTKYNEEFLLYLAGFVDGDGSIVAQIKPNQGCKFKHQLCLTFQVTQKTQRRWFLDKLVDEIGVGYVADSGSVSNYRLSKIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQHCKFKHQLALTFYVTQKTQRRWFLDKLVDRIGVGYVYDSGSVSRYVLSEIKPLHNFLTQLQPFLKLKQKQANLALKIIEQLPSAKESPDKFLEVCTWVDQVAALNDSKTRKTTSETVRAVLDSLSEKKKSSP

SEQ ID NO: 74 - protein, 354 aa, artificial sequence
  SCOH-DMD24-pCLS4713
MANTKYNEEFLLYLAGFVDGDGSIVAQIKPNQGCKFKHQLCLTFQVTQKTQRRWFLDKLVDEIGVGYVADSGSVSNYRLSKIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQHCKFKHQLALTFYVTQKTQRRWFLDKLVDRIGVGYVYDRGSVSDYVLSEIKPLHNFLTQLQPFLKLKQKQANLALKIIEQLPSAKESPDKFLEVCTWVDQVAALNDSKTRKTTSETVRAVLDSLSEKKKSSP

SEQ ID NO: 75 - protein, 354 aa, artificial sequence
  SCOH-DMD24-pCLS3403
MANTKYNEEFLLYLAGFVDGDGSIVAQIKPNQGCKFKHQLCLTFQVTQKTQRRWFLDKLVDEIGVGYVADSGSVSNYRLSKIKPLHNFLTQLQPFLELKQKQANLALKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQHCKFKHQLALTFYVTQKTQRRWFLDKLVDRIGVGYVYDSGSVSRYVLSKIKPLHNFLTQLQPFLKLKQKQANLALKIIEQLPSAKESPDKFLEVCTWVDQVAALNDSKTRKTTSETVRAVLDSLSEKKKSSP

SEQ ID NO: 76 - protein, 354 aa, artificial sequence
  SCOH-DMD24-pCLS3404
MANTKYNEEFLLYLAGFVDGDGSIVAQIKPNQGCKFKHQLCLTFQVTQKTQRRWFLDKLVDEIGVGYVADSGSVSNYRLSKIKPLHNFLTQLQPFLELKQKQANLALKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQHCKFKHQLALTFYVTQKTQRRWFLDKLVDRIGVGYVYDSGSVSRYVLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 77 - protein, 354 aa, artificial sequence
  SCOH-DMD24-pCLS4327
MANTKYNEEFLLYLAGFVDGDGSIVAQIKPNQGCKFKHQLCLTFQVTQKTQRRWFLDKLVDEIGVGYVADSGSVSNYRLSKIKPLHNFLTQLQPFLELKQKQANLALKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQHCKFKHQLALTFYVTQKTQRRWFLDKLVDRIGVGYVYDRGSVSDYVLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 78 - protein, 354 aa, artificial sequence
  SCOH-DMD31-pCLS3627
MANTKYNEEFLLYLAGFVDGDGSIIAQIKPHQTCKFKHRLSLTFAVTQKTRRRWFLDKLVDEIGVGYVRDSGSVSYYTLSEIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIVAQIKPNQSRKFKHQLELTFIVTQKTQRRWFLDKLVGRIGVGYVRDSGSVSNYRLSEIKPLHNFLTQLQPFLKLKQKQANLVLKIIEQLPSAKESPDKFLEVCTWADQVAALNDSKTRKTTSETVRAVLDSLGEKKKSSP

SEQ ID NO: 79 - protein, 354 aa, artificial sequence
  SCOH-DMD31-pCLS3628
MANTKYNEEFLLYLAGFVDGDGSIIAQIKPHQTCKFKHRLSLTFAVTQKTRRRWFLDKLVDEIGVGYVRDSGSVSYYTLSKIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIVAQIKPNQSRKFKHQLELTFIVTQKTQRRWFLDKLVGRIGVGYVRDSGSVSNYRLSEIKPLHNFLTQLQPFLKLKQKQANLALKIIEQLPSAKESPDKFLEVCTWADQVAALNDSKTRKTTSETVRAVLDSLGEKKKSSP

SEQ ID NO: 80 - protein, 354 aa, artificial sequence
  SCOH-DMD31-pCLS3629
MANTKYNEEFLLYLAGFVDGDGSIIAQIKPHQTCKFKHRLSLTFAVTQKTRRRWFLDKLVDEIGVGYVRDSGSVSYYTLSKIKPLHNFLTQLQPFLELKQKQANLALKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIVAQIKPNQSRKFKHQLELTFIVTQKTQRRWFLDKLVGRIGVGYVRDSGSVSNYRLSKIKPLHNFLTQLQPFLKLKQKQANLALKIIEQLPSAKESPDKFLEVCTWADQVAALNDSKTRKTTSETVRAVLDSLGEKKKSSP

SEQ ID NO: 81 - protein, 354 aa, artificial sequence
  SCOH-DMD31-pCLS3630
MANTKYNEEFLLYLAGFVDGDGSIIAQIKPHQTCKFKHRLSLTFAVTQKTQRRWFLDKLVDEIGVGYVRDSGSVSYYTLSKIKPLHNFLTQLQPFLELKQKQANLVLKIIEQLPSAKESPDKFLEVCTWVDQIAALNDSKTRKTTSETVRAVLDSLSEKKKSSPAAGGSDKYNQALSKYNQALSKYNQALSGGGGSNKKFLLYLAGFVDSDGSIIAQIKPNQSRKFKHQLELTFLVTQKTQRRWFLDKLADRVGVGYVRDAGSVSNYRLSKIKPLHNFLTQLQPFLKLKQKQANLVLKIIEQLPSAKESPDKFLEVCTWADQIAALNDSKTRKTTSETVRAVLDSLGEKKKSSP

SEQ ID NO: 82 - protein, 354 aa, artificial sequence
  SCOH-DMD31-pCLS3631
MANTKYNEEFLLYLAGFVDGDGSIIAQIKPHQTCKFKHRLSLTFAVTQKTQRRWFLDKLVDEIGVGYVRDSGSVSYYTLSKIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQSRKFKHQLELTFLVTQKTQRRWFLDKLADRVGVGYVRDAGSVSNYRLSKIKPLHNFLTQLQPFLKLKQKQANLVLKIIEQLPSAKESPDKFLEVCTWADQVAALNDSKTRKTTSETVRAVLDSLGEKKKSSP

SEQ ID NO: 83 - protein, 354 aa, artificial sequence
  SCOH-DMD31-pCLS3632
MANTKYNEEFLLYLAGFVDGDGSIIAQIKPHQTCKFKHRLSLTFAVTQKTQRRWFLDKLVDEIGVGYVRDSGSVSYYTLSKIKPLHNFLTQLQPFLELKQKQANLALKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQSRKFKHQLELTFLVTQKTQRRWFLDKLADRVGVGYVRDAGSVSNYRLSKIKPLHNFLTQLQPFLKLKQKQANLALKIIEQLPSAKESPDKFLEVCTWADQVAALNDSKTRKTTSETVRAVLDSLGEKKKSSP

SEQ ID NO: 84 - protein, 354 aa, artificial sequence
  SCOH-DMD31-pCLS3633
MANTKYNEEFLLYLAGFVDGDGSIIAQIKPHQTCKFKHRLSLTFAVTQKTQRRWFLDKLVDEIGVGYVRDSGSVSYYTLSKIKPLHNFLTQLQPFLELKQKQANLALKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQSRKFKHQLELTFLVTQKTQRRWFLDKLADRVGVGYVRDAGSVSNYRLSKIKPLHNFLTQLQPFLKLKQKQANLVLKIIEQLPSAKESPDKFLEVCTWADQVAALNDSKTRKTTSETVRAVLDSLGEKKKSSP

SEQ ID NO: 85 - protein, 354 aa, artificial sequence
  SCOH-DMD33-pCLS3326
MANTKYNEEFLLYLAGFVDGDGSIIAQIKPRQANKFKHQLELTFTVTQKTQRRWFLDKLVDEIGVGYVYDSGSVSKYQLSKIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQGHKFKHQLSLTFKVTQKTQRRWFLDKLVDRIGVGYVYDSGSVSYYNLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 86 - protein, 354 aa, artificial sequence
  SCOH-DMD33-pCLS3327
MANTKYNEEFLLYLAGFVDGDGSIIAQIKPRQANKFKHQLELTFTVTQKTQRRWFLDKLVDEIGVGYVYDSGSVSKYQLSKIKPLHNFLTQLQPFLELKQKQANLVLKIIEQLPSAKESPDKFLEVCTWVDQIAALNDSKTRKTTSETVRAVLDSLSEKKKSSPAAGGSDKYNQALSKYNQALSKYNQALSGGGGSNKKFLLYLAGFVDSDGSIIAQIKPNQGHKFKHQLSLTFKVTQKTQRRWFLDKLVDRIGVGYVYDSGSVSYYNLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 87 - protein, 354 aa, artificial sequence
  SCOH-DMD33-pCLS3328
MANTKYNEEFLLYLAGFVDGDGSIIAQIKPRQANKFKHQLELTFTVTQKTQRRWFLDKLVDEIGVGYVYDSGSVSKYQLSKIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQGHKFKHQLSLTFKVTQKTQRRWFLDKLVDRIGVGYVYDSGSVSYYNLSEIKPLHNFLTQLQPFLKLKQKQANLALKIIEQLPSAKESPDKFLEVCTWVDQVAALNDSKTRKTTSETVRAVLDSLSEKKKSSP

SEQ ID NO: 88 - protein, 354 aa, artificial sequence
  SCOH-DMD33-pCLS3329
MANTKYNEEFLLYLAGFVDSDGSIIAQIKPRQANKFKHQLELTFQVTQKTQRRWFLDKLVDEIGVGYVRDSGSVSRYTLSKIKPLHNFLTQLQPFLELKQKQANLVLKIIEQLPSAKESPDKFLEVCTWVDQIAALNDSKTRKTTSETVRAVLDSLSEKKKSSPAAGGSDKYNQALSKYNQALSKYNQALSGGGGSNKKFLLYLAGFVDGDGSIIAQIKPNQGHKFKHQLSLTFKVTQKTQRRWFLDKLVDRIGVGYVYDSGSVSDYKLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 89 - protein, 354 aa, artificial sequence
  SCOH-DMD33-pCLS3330
MANTKYNEEFLLYLAGFVDSDGSIIAQIKPRQANKFKHQLELTFQVTQKTQRRWFLDKLVDEIGVGYVRDSGSVSRYTLSKIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDGDGSIIAQIKPNQGHKFKHQLSLTFKVTQKTQRRWFLDKLVDRIGVGYVYDSGSVSDYKLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 90 - protein, 354 aa, artificial sequence
  SCOH-DMD33-pCLS3331
MANTKYNEEFLLYLAGFVDSDGSIIAQIKPRQANKFKHQLELTFQVTQKTQRRWFLDKLVDEIGVGYVRDSGSVSRYTLSKIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDGDGSIIAQIKPNQGHKFKHQLSLTFKVTQKTQRRWFLDKLVDRIGVGYVYDSGSVSDYKLSEIKPLHNFLTQLQPFLKLKQKQANLALKIIEQLPSAKESPDKFLEVCTWVDQVAALNDSKTRKTTSETVRAVLDSLSEKKKSSP

SEQ ID NO: 91 - protein, 354 aa, artificial sequence
  SCOH-DMD33-pCLS3332
MANTKYNEEFLLYLAGFVDSDGSIIAQIKPRQANKFKHQLELTFQVTQKTQRRWFLDKLVDEIGVGYVRDSGSVSRYTLSKIKPLHNFLTQLQPFLELKQKQANLALKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDGDGSIIAQIKPNQGHKFKHQLSLTFKVTQKTQRRWFLDKLVDRIGVGYVYDSGSVSDYKLSKIKPLHNFLTQLQPFLKLKQKQANLALKIIEQLPSAKESPDKFLEVCTWVDQVAALNDSKTRKTTSETVRAVLDSLSEKKKSSP

SEQ ID NO: 92 - protein, 354 aa, artificial sequence
  SCOH-DMD33-pCLS3333
MANTKYNEEFLLYLAGFVDGDGSIIAQIKPRQANKFKHQLELTFQVTQKTQRRWFLDKLVDEIGVGYVRDSGSVSRYTLSKIKPLHNFLTQLQPFLELKQKQANLVLKIIEQLPSAKESPDKFLEVCTWVDQIAALNDSKTRKTTSETVRAVLDSLSEKKKSSPAAGGSDKYNQALSKYNQALSKYNQALSGGGGSNKKFLLYLAGFVDSDGSIIAQIKPNQGHKFKHQLSLTFKVTQKTQRRWFLDKLVDRIGVGYVYDSGSVSDYKLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 93 - protein, 354 aa, artificial sequence
  SCOH-DMD33-pCLS3335
MANTKYNEEFLLYLAGFADSDGSIIAQIKPRQANKFKHQLELTFQVTQKTQRRWFLDKLVDEIGVGYVRDSGSVSRYTLSKIKPLHNFLTQLQPFLELKQKQANLVLKIIEQLPSAKESPDKFLEVCTWVDQIAALNDSKTRKTTSETVRAVLDSLSEKKKSSPAAGGSDKYNQALSKYNQALSKYNQALSGGGGSNKKFLLYLAGFVDGDGSIIAQIKPNQGHKFKHQLSLTFKVTQKTQRRWFLDELVDRIGVGYVYDSGSVSYYNLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 94 - protein, 354 aa, artificial sequence
  SCOH-DMD33-pCLS3336
MANTKYNEEFLLYLAGFADSDGSIIAQIKPRQANKFKHQLELTFQVTQKTQRRWFLDKLVDEIGVGYVRDSGSVSRYTLSKIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDGDGSIIAQIKPNQGHKFKHQLSLTFKVTQKTQRRWFLDELVDRIGVGYVYDSGSVSYYNLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 95 - protein, 354 aa, artificial sequence
  SCOH-DMD33-pCLS3340
MANTKYNEEFLLYLAGFADGDGSIIAQIKPRQANKFKHQLELTFQVTQKTQRRWFLDKLVDEIGVGYVRDSGSVSRYTLSKIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQGHKFKHQLSLTFKVTQKTQRRWFLDELVDRIGVGYVYDSGSVSYYNLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 96 - protein, 354 aa, artificial sequence
  SCOH-DMD35-pCLS4901
MANTKYNEEFLLYLAGFVDGDGSIIAQIKPNQSCKFKHALSLTFQVTQKTQRRWLLDKLVDEIGVGYVRDSGSVSHYYLSEIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIVAQIKPNQKYKFKHQLSLTFRVAQKTQRRWFLDKLVDRIGVGHVYDSGSVSYYNLSEIKPLHNFLTQLQPFLKLKQKQANLVLKIIEQLPSTKESPDKFLEVCTWVDQVAALNDSKTRKTTSETVRAVLDSLSEKKKSSP

SEQ ID NO: 97 - protein, 354 aa, artificial sequence
  SCOH-DMD35-pCLS4902
MANTKYNEEFLLYLAGFVDGDGSIIAQIKPNQSCKFKHALSLTFQVTQKTQRRWLLDKLVDEIGVGYVRDSGSVSHYYLSKIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIVAQIKPNQKYKFKHQLSLTFRVAQKTQRRWLLDKLVDRIGVGHVYDSGSVSYYNLSEIKPLHNFLTQLQPFLKLKQKQANLVLKIIERLPSAKESPDRFLEVCTWVDQVAALNDSRTRKTTSETVRAVLDSLSEKKKSSP

SEQ ID NO: 98 - protein, 354 aa, artificial sequence
  SCOH-DMD35-pCLS4903
MANTKYNEEFLLYLAGFVDGDGSIIAQIKPNQSCKFKHALSLTFQVTQKTQRRWLLDKLVDEIGVGYVRDSGSVSHYYLSKIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIVAQIKPNQKYKFKHQLSLTFRVAQKTQRRWFLDKLVDRIGVGHVYDSGSVSYYNLSEVKPLHNFLTQLQPFLKLKQKQANLVLKIIEQLPSAKESPDKFLEVCTWVDQVAALNDSKTRKTTSETVRAVLDSLSEKKKSSP

SEQ ID NO: 99 - protein, 354 aa, artificial sequence
  SCOH-DMD35-pCLS4904
MANTKYNEEFLLYLAGFVDGDGSIIAQIKPNQSCKFKHALSLTFQVTQKTQRRWLLDKLVDEIGVGYVRDSGSVSHYYLSKIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIVAQIKPNQKYKFKHQLSLTFRVAQKTQRRWFLDKLVDRIGVGHVYDSGSVSYYNLSEIKPLHNFLTQLQPFLKLKQKQANLVLKIIEQLPSTKESPDKFLEVCTWVDQVAALNDSKTRKTTSETVRAVLDSLSEKKKSSP

SEQ ID NO: 100 - protein, 354 aa, artificial sequence
  SCOH-DMD37-pCLS4606
MANTKYNEEFLLYLAGLVDGDGSIIAQIKPTQSYKFKHQLRLTFQVTQKTQRRWLLDKLVDEIGVGYVRDSGSVSHYYLSEIKPLHNFLTQLQPFLELKQKQANLVLKIIEQLPSAKESPDKFLEVCTWVDQIAALNDSKTRKTTSETVRAVLDSLSEKKKSSPAAGGSDKYNQALSKYNQALSKYNQALSGGGGSNKKFLLYLAGFVDSDGSIIAQIKPGQSYKFKHRLSLTFTVTQKTQRRWFLDKLVDRIGVGYVRDQGSVSYYILSEIKPLHNFLTQLQPFLRLKQKQANLVLKIIEQLPSAKESPDKFLEVCTWVDQIAALNDSKTRKTTSETVRAVLDSLSEKKKSSP

SEQ ID NO: 101 - protein, 354 aa, artificial sequence
  SCOH-DMD37-pCLS4607
MANTKYNEEFLLYLAGLVDGDGSIIAQIKPTQSYKFKHQLRLTFQVTQKTQRRWLLDKLVDEIGVGYVRDSGSVSHYYLSEIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPGQSYKFKHRLSLTFTVTQKTQRRWFLDKLVDRIGVGYVRDQGSVSYYILSEIKPLHNFLTQLQPFLRLKQKQANLVLKIIEQLPSAKESPDKFLEVCTWVDQVAALNDSKTRKTTSETVRAVLDSLSEKKKSSP

SEQ ID NO: 102 - protein, 354 aa, artificial sequence
  SCOH-DMD37-pCLS4608
MANTKYNEEFLLYLAGLVDGDGSIIAQIKPTQSYKFKHQLRLTFQVTQKTQRRWLLDKLVDEIGVGYVRDSGSVSHYYLSKIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPGQSYKFKHRLSLTFTVTQKTQRRWFLDKLVDRIGVGYVRDQGSVSYYILSEIKPLHNFLTQLQPFLRLKQKQANLVLKIIEQLPSAKESPDKFLEVCTWVDQVAALNDSKTRKTTSETVRAVLDSLSEKKKSSP

SEQ ID NO: 103 - protein, 354 aa, artificial sequence
  SCOH-DMD37-pCLS4609
MANTKYNEEFLLYLAGLVDSDGSIIAQIKPTQSYKFKHQLRLTFQVTQKTQRRWLLDKLVDEIGVGYVRDSGSVSHYYLSEIKPLHNFLTQLQPFLELKQKQANLVLKIIEQLPSAKESPDKFLEVCTWVDQIAALNDSKTRKTTSETVRAVLDSLSEKKKSSPAAGGSDKYNQALSKYNQALSKYNQALSGGGGSNKKFLLYLAGFVDGDGSIIAQIKPGQSYKFKHRISLTFAVTQKTQRRWFLDKLVDRIGVGYVRDQGSVSNYILSEIKPLHNFLTQLHPFLKLKQKQADLVLKIIEQLPSAKESPGKFLEVCTWVDQIAALNDSKKRKTTSESVRAVLDSLSEKKKSSP

SEQ ID NO: 104 - protein, 354 aa, artificial sequence
  SCOH-DMD37-pCLS4610
MANTKYNEEFLLYLAGLVDSDGSIIAQIKPTQSYKFKHQLRLTFQVTQKTQRRWLLDKLVDEIGVGYVRDSGSVSHYYLSEIKPLHNFLTQLQPFLELKQKQANLVLKIIEQLPSAKESPDKFLEVCTWVDQIAALNDSKTRKTTSETVRAVLDSLSEKKKSSPAAGGSDKYNQALSKYNQALSKYNQALSGGGGSNKKFLLYLAGFVDGDGSIIAQIKPGQSYKFKHRISLTFAVTQKTQRRWFLDKLVDRIGVGYVRDQGSVSNYILSEIKPLHNFLTQLHPFLKLKQKQADLVLKIIEQLPSAKESPGKFLEVCTWVDQVAALNDSKKRKTTSESVRAVLDSLSEKKKSSP

SEQ ID NO: 105 - protein, 354 aa, artificial sequence
  SCOH-DMD37-pCLS4611
MANTKYNEEFLLYLAGLVDSDGSIIAQIKPTQSYKFKHQLRLTFQVTQKTQRRWLLDKLVDEIGVGYVRDSGSVSHYYLSEIKPLHNFLTQLQPFLELKQKQANLVLKIIEQLPSAKESPDKFLEVCTWVDQIAALNDSKTRKTTSETVRAVLDSLSEKKKSSPAAGGSDKYNQALSKYNQALSKYNQALSGGGGSNKKFLLYLAGFVDGDGSIIAQIKPGQSYKFKHRISLTFAVTQKTQRRWFLDKLVDRIGVGYVRDQGSVSNYILSKIKPLHNFLTQLHPFLKLKQKQADLVLKIIEQLPSAKESPGKFLEVCTWVDQVAALNDSKKRKTTSESVRAVLDSLSEKKKSSP

SEQ ID NO: 106 - DNA, 51 bp, Artificial
  DMD 21 cloning oligonucleotide
tggcatacaagtttgaaacctcaagtaccaaatgtaaacaatcgtctgtca

SEQ ID NO: 107 - DNA, 51 bp, Artificial
  DMD 24 cloning oligonucleotide
tggcatacaagttttttacctattttaagtcagatacacaatcgtctgtca

SEQ ID NO: 108 - DNA, 51 bp, Artificial
  DMD 31 cloning oligonucleotide
tggcatacaagtttaatgtctgatgttcaatgtgttgacaatcgtctgtca

SEQ ID NO: 109 - DNA, 51 bp, Artificial
  DMD 33 cloning oligonucleotide
tggcatacaagtttaaatcctgccttaaagtatctcatcaatcgtctgtca

SEQ ID NO: 110 - DNA, 51 bp, Artificial
  DMD 35 cloning oligonucleotide
tggcatacaagttttctttatgttttaaagtatattcccaatcgtctgtca

SEQ ID NO: 111 - DNA, 51 bp, Artificial
  DMD 37 cloning oligonucleotide
tggcatacaagtttgaatcctgttgttcatcatcctagcaatcgtctgtca

SEQ ID NO: 112 - DNA, 10105 bp, Artificial
  pIM_DMD_MCS
ggcgcgccggtaccgagcctgcaggttagacgcgtgtcacccgggttaagatctcttaaggaattcggccggccttgtttaaaccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatctgctagggagctcgggcccccgcggcagcacagtggtcgacggcgcgtaatgtaaaagaaaggctatgagcacagtatcttataatagcattccatgaaagttttaaattggatttttgtgtgtgtttaaataacatgtcttattatctctgttaacaatgtacagctttttaaaaaccaaaatgaagactgtacttgttgtttttgatcagaatgaagacaatgagggtactgtaaaagaattgttgcaaagaggagacaacttacaacaaagaatcacagatgagagaaagcgagaggaaataaagataaaacagcagctgttacagacaaaacataatgctctcaaggtattagagctaaaattataatataccttgcctgtggtttttttttaatatatagggtaaaatataatgtgcattaataaaatctgcttcagactcttagtcatcagaaactcactttttctgttcaatgtgtatgctttatttaacatttttgagtggtatttgattttgaacgatgtgttgcttaaaagttatggctttttaaagacatatttttctaagtagtaattgaaagatggaatgcattcatagtacttatacatttaattttctaatgtgctttatttttatcagctactttatattgaaaaaaacctgcctaactgccagaataaccgatctacatgtatccattattcctgctttctttacttaattgattttgcttgcattaggtccaattttttggtaacctgatacttgtttaataagccctagattcttttatttatttttaaaactttattatgcctttaagaaagtcactctttgcaactatctacaaagtcaaatttcatttatgtaggaatcctaaaaaataatcttagtgcccttgaataagaactgctactttcatattaagcagtgtagtgtatggcttcaagtccccaggtgactcatctggtttgtgcaggaagagcaggataaacttaatttttctataataatatctaggaagttcttgagactagacatgtggaaagttttgtgaaactttttttttgtatatgctttgcaataatcactatttgcatgcatggctgattttctaatacttttaattgctgagcttttccttgtaaatcactcttttgagctatgtagttatgaaggtggatccacctagcaggtagatggagtctgctttctacatacaaataagagaataaaacctttccccctatagtcaaccaaataaacactgctgagaatgtaattacacaatattgtgcctgcaaggaatccacaaggaaagtctaattggccagcaggtgttctgaaggtaactgctgataaatatagtaaacagaaaaggcctcttcctcgagggagtgcaaaagcacaccataatgtgtctaggcgcgtttaaagcttgtacatcgatgcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacagctgagatcaccggcgaaggagggccaccatatggcttcttaccctggacaccagcatgcttctgcctttgaccaggctgccagatccaggggccactccaacaggagaactgccctaagacccagaagacagcaggaagccactgaggtgaggcctgagcagaagatgccaaccctgctgagggtgtacattgatggacctcatggcatgggcaagaccaccaccactcaactgctggtggcactgggctccagggatgacattgtgtatgtgcctgagccaatgacctactggagagtgctaggagcctctgagaccattgccaacatctacaccacccagcacaggctggaccagggagaaatctctgctggagatgctgctgtggtgatgacctctgcccagatcacaatgggaatgccctatgctgtgactgatgctgttctggctcctcacattggaggagaggctggctcttctcatgcccctccacctgccctgaccctgatctttgacagacaccccattgcagccctgctgtgctacccagcagcaaggtacctcatgggctccatgaccccacaggctgtgctggcttttgtggccctgatccctccaaccctccctggcaccaacattgttctgggagcactgcctgaagacagacacattgacaggctggcaaagaggcagagacctggagagagactggacctggccatgctggctgcaatcagaagggtgtatggactgctggcaaacactgtgagatacctccagtgtggaggctcttggagagaggactggggacagctctctggaacagcagtgccccctcaaggagctgagccccagtccaatgctggtccaagaccccacattggggacaccctgttcaccctgttcagagcccctgagctgctggctcccaatggagacctgtacaatgtgtttgcctgggctctggatgttctagccaagaggctgaggtccatgcatgtgttcatcctggactatgaccagtcccctgctggatgcagagatgctctgctgcaactaacctctggcatggtgcagacccatgtgaccacccctggcagcatccccaccatctgtgacctagccagaacctttgccagggagatgggagaggccaactaaaattcgctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatcatttaaatgttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggatctcgagcggccgcggcgcgtctgtggcattcttaacctttacaattaccagtgctagagccttctcagacaatgcttgaaagtgacatcagaagacaatatataaattttaggtaataagtgtgtgaagcaataggagaactattcccaccaatcttagttcccacttcctggagctgcctcaatcaatactccccatgtatggggtttggtggccttgccaactcagcttgtggaaagacggaagaggctttccaagttgctgagttacagcaatctctacttcaggtcaaagaagaaactagagggcaccaagatgaaaaaaagtactgattacctcttgggctgaccccaaccctgccctagagccctcaactggtatagggaaagaagtgaagtgggttttttaattttttgttgtttttgtcctcagccctagaaaatgctacagacttttacaacaccttttaaaataagtaaagtcttttacagactataagtatatgatgtgtctatattatgataagtatataatgtgtctatattatgccgtatgtttctttagattattgaccagtctctgacaaaaataatccctttcccccatacaaaatgaatcagtcctcagttgcgtgttgaagtttatgcattatagatttaaatatataacatctaattccttgaattgtaatacacacatacaaatacttgtgacttttgaaatttattatttgttttccttatgtcatcagtgtaccttgagtaccattacaatgtgagactacttgatacaaagttttcacattctgagttgaaagaaagaaaactaaaggtgaaaaatcacatggtcatatatgcgactatattagaccagcatttattcaaaacattttatttctaagtaaagaaaattaagatattgttcttgcagcctaaaggaacaaaatacccattgaaaatgtttaaagccattttccagtccatattttagtattatttgatcgtatttaaattaacatgcatatatgacatattttagaaattatttgatggtaacttagcacatgatttttattaatatcctgatatttctcctattaatattgagcatttaggtttcagaaatgaagcaggagttattagataggaattagtgtacatttaaatcgaaatattttcatgtagttgtgtgttaatattttcagatttgctgttatctcagtcacaaatacacatctgtattcctttatggatcagttaacgttataaatgataactttagctcatttctctaatgttttaatttctagaactacactaaaaaagccaaaagaatactttcacagtaattcatgacttgtctgacatgatgaagtaccacactcccccatctttctacaggtgcccctaaaaatgtgttctttacaactagatgagaggaaacttatttgaaacctcggacgcgccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgattaattaacaggactgaccgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacaggatccactagcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcctta

SEQ ID NO: 113 - DNA, 12670 bp, Artificial
  pIM_DMD_Luc
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaagctagctgacgggatcaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaggccggcctgcaggaatgtaaaagaaaggctatgagcacagtatcttataatagcattccatgaaagttttaaattggatttttgtgtgtgtttaaataacatgtcttattatctctgttaacaatgtacagctttttaaaaaccaaaatgaagactgtacttgttgtttttgatcagaatgaagacaatgagggtactgtaaaagaattgttgcaaagaggagacaacttacaacaaagaatcacagatgagagaaagcgagaggaaataaagataaaacagcagctgttacagacaaaacataatgctctcaaggtattagagctaaaattataatataccttgcctgtggtttttttttaatatatagggtaaaatataatgtgcattaataaaatctgcttcagacttagtcatcagaaactcactttttctgttcaatgtgtatgctttatttaacatttttgagtggtatttgattttgaacgatgtgttgcttaaaagttatggctttttaaagacatatttttctaagtagtaattgaaagatgggaatgcattcatagtacttatacatttaattttctaatgtgctttatttttatcagctactttatattgaaaaaaacctgcctaactgccagaataaccgatctacatgtatccattattcctgctttctttacttaattgattttgcttgcattaggtccaattttttggtaacctgatacttgtttaataagccctagattcttttatttatttttaaaactttattatgcctttaagaaagtcactctttgcaactatctacaaagtcaaatttcatttatgtaggaatcctaaaaaataatcttagtgcccttgaataagaactgctactttcatattaagcagtgtagtgtatggcttcaagtccccaggtgactcatctggtttgtgcaggaagagcaggataaacttaatttttctataataatatctaggaagttcttgagactagacatgtggaaagttttgtgaaactttttttttgtatatgctttgcaataatcactatttgcatgcatggctgattttctaatacttttaattgctgagcttttccttgtaaatcactcttttgagctatgtagttatgaaggtggatccacctagcaggtagatggagtctgctttctacatgcaaataagagaataaaacctttccccctatagtcaaccaaataaacactgctgagaatgtaattacacaatattgtgcctgcaaggaatccacaaggaaagtctaattggccagcaggtgttctgaaggtaactgctgataaatatagtaaacagaaaaggcctcttcctcgagggagtgcaaaagcacaccataatgtgtctacctgcaggttcgaaaagggcgaattcgcgtttaaagcttgtacatcgatgcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacagctgagatcaccggcgaaggagggccaccatggcttcttaccctggacaccagcatgcttctgcctttgaccaggctgccagatccaggggccactccaacaggagaactgccctaagacccagaagacagcaggaagccactgaggtgaggcctgagcagaagatgccaaccctgctgagggtgtacattgatggacctcatggcatgggcaagaccaccaccactcaactgctggtggcactgggctccagggatgacattgtgtatgtgcctgagccaatgacctactggagagtgctaggagcctctgagaccattgccaacatctacaccacccagcacaggctggaccagggagaaatctctgctggagatgctgctgtggtgatgacctctgcccagatcacaatgggaatgccctatgctgtgactgatgctgttctggctcctcacattggaggagaggctggctcttctcatgcccctccacctgccctgaccctgatctttgacagacaccccattgcagccctgctgtgctacccagcagcaaggtacctcatgggctccatgaccccacaggctgtgctggcttttgtggccctgatccctccaaccctccctggcaccaacattgttctgggagcactgcctgaagacagacacattgacaggctggcaaagaggcagagacctggagagagactggacctggccatgctggctgcaatcagaagggtgtatggactgctggcaaacactgtgagatacctccagtgtggaggctcttggagagaggactggggacagctctctggaacagcagtgccccctcaaggagctgagccccagtccaatgctggtccaagaccccacattggggacaccctgttcaccctgttcagagcccctgagctgctggctcccaatggagacctgtacaatgtgtttgcctgggctctggatgttctagccaagaggctgaggtccatgcatgtgttcatcctggactatgaccagtcccctgctggatgcagagatgctctgctgcaactaacctctggcatggtgcagacccatgtgaccacccctggcagcatccccaccatctgtgacctagccagaacctttgccagggagatgggagaggccaactaaacctgagctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatcatttaaatgttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggatctcgataacaaaaaaccccgccccggcggggttttttgttagcggccgcggcgcgccgataagtctccaactgaattttcagagcacatattacacattttatgattctaatgaattttataaatgcagaatattcttggccttttattacaaatgctgaattactatggtatggtaatataggagcacttacggtttctaataaatagcccagcaattttcttttgacctgacctacttttgaacttatcaagggcaaaatgtaattggaaaaaagagtcgaatcataaaataaactaaaaatttaataaaatactcaactatcatagatgactgacattttgtatcagcaatgtttttggtagtataacatttatttcaatgaagagagaataatgcataatatgaacattatttattacaatttcccacaaagttgttttaccaaatagttgtttagttagtacattatatgtctaaaatgtgtataaattcatgctgcttatttcatgggagtaaagaaatatgaccatgtgtgctcttcccaacctcagaatattctccttcaatcccaactaatttcaaagtatctactacatttctctctggcttgaatatttgtacatcacatccttattaactgactttttttttctctcttcatcctccttttcacaattttttcccctctttcactgccatcacctctgtggcattcttaacctttacaattaccagtgctagagccttctcagacaatgcttgaaagtgacatcagaagacaatatataaattttaggtaataagtgtgtgaagcaataggagaactattcccaccaatcttagttcccacttcctggagctgcctcaatcaatactccccatgtatggggtttggtggccttgccaactcagcttgtggaaagacggaagaggctttccaagttgctgagttacagcaatctctacttcaggtcaaagaagaaactagagggcaccaagatgaaaaaaagtactgattacctcttgggctgaccccaactctgccctagagccctcaactggtatagggaaagaagtgaagtgggttttttaattttttgttgtttttgtcctcagccctagaaaatgctacagacttttacaacaccttttaaaataagtaaagtcttttacagactataagtatatgatgtgtctatattatgataagtatataatgtgtctatattatgccgtatgtttctttagattattgaccagtctctgacaaaaataatccctttcccccatacaaaatgaatcagtcctcagttgcgtgttgaagtttatgcattatagatttaaatatataacatctaattccttgaattgtaatacacacatacaaatacttgtgacttttgaaatttattatttgttttccttatgtcatcagtgtaccttgagtaccattacaatgtgagactacttgatacaaagttttcacattctgagttgaaagaaagaaaactaaaggtgaaaaatcacatggtcatatatgcgactatattagaccagcatttattcaaaacattttatttctaagtaaagaaaattaagatattgttcttgcagcctaaaggaacaaaatacccattgaaaatgtttaaagccattttccagtccatattttagtattatttgatcgtatttaaattaacatgcatatatgacatattttagaaattatttgatggtaacttagcacatgatttttattaatatcctgatatttctcctattaatattgagcatttaggtttcagaaatgaagcaggagttattagataggaattagtgtacatttaaatcaaaatattttcatgtagttgtgtgttaatattttcagatttgctgttatctcagtcacaaatacacatctgtattcctttatggatcagttaacattataaatgataactttagctcatttctctaatgttttaatttctagaactacactaaaaaagccaaaagaatactttcacagtaattcatgacttgtctgacatgatgaagtaccacactcccccatctttctacaggtgcccctaaaaatgtgttctttacaactagatgggaggaaacttattttgaaacctcggcgcgccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgattaattaacaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgacgcgaattcgcccttgcgcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccttaggcgcggctagggataacagggtaatatcgcgccagatctgtacattcgaagatatcttaattaagcggccgctcgagtctagagggcccgtttaaacccgctgatcagccgcgaattcactagtgattgcagaattc

SEQ ID NO: 114 - DNA, 30 bp, Artificial
  F_HS2_PCRsc PCR screen forward primer
ccttccttgaccctggaaggtgccactccc

SEQ ID NO: 115 - DNA, 31 bp, Artificial
  R_HS2_PCRsc PCR screen reverse primer
ttaaacactgctattcagtaggacacacacc

SEQ ID NO: 116 - protein, 354 aa, artificial sequence
  SCOH-DMD21-pCLS5353
MANTKYNEEFLLYLAGFVDGDGSIIAQIKPRQSYKFKHQLELTFTVGQKTQRRWFLDKLVDEIGVGYVTDSGSMSAYRLSKIKPLHNFLTQLQPFLELKQKQANLVLKIIEQLPSAKESPDKFLEVCTWVDQVAALNDSKTRKTTSETVRAVLDNLSEKKKSSPAAGGSDKYNQALSKYNQALSKYNQALSGGGGSNKKFLLYLAGFVDSDGSIIAQIRPNQSAKFKHYLQLTFRVTQKTQRRWFLDKLVDRIGVGYVYDSGSVSDYILSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 117 - protein, 354 aa, artificial sequence
  SCOH-DMD21-pCLS5354
MANTKYNEEFLLYLAGFVDSDGSIIAQIKPRQSYKFKHQLELTFTVGQKTQRRWFLDKLVDEIGVGYVTDSGSMSAYRLSKIKPLHNFLTQLQPFLELKQKQANLVLKIIEQLPSAKESPDKFLEVCTWVDQVAALNDSKTRKTTSETVRAVLDNLSEKKKSSPAAGGSDKYNQALSKYNQALSKYNQALSGGGGSNKKFLLYLAGFVDGDGSIIAQIRPNQSAKFKHYLQLTFRVTQKTQRRWFLDKLVDRIGVGYVYDSGSVSDYILSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 118 - protein, 354 aa, artificial sequence
  SCOH-DMD21-pCLS5355
MANTKYNEEFLLYLAGFVDGDGSIIAQIKPRQSYKFKHQLELTFTVGQKTRRRWFLDKLVDEIGVGYVTDSGSMSAYRLSEIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIRPNQSAKFKHYLQLTFRVTQKTQRRWFLDKLVDRIGVGYVYDSGSVSDYILSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 119 - protein, 354 aa, artificial sequence
  SCOH-DMD21-pCLS5356
MANTKYNEEFLLYLAGFVDSDGSIIAQIKPRQSYKFKHQLELTFTVGQKTRRRWFLDKLVDEIGVGYVTDSGSMSAYRLSEIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDGDGSIIAQIRPNQSAKFKHYLQLTFRVTQKTQRRWFLDKLVDRIGVGYVYDSGSVSDYILSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 120 - protein, 25 aa, Artificial
  BQY peptidic linker
GDSSVSNSEHIAPLSLPSSPPSVGS

SEQ ID NO: 121 - protein, 349 aa, artificial sequence
  SCOH-DMD35-pCLS6601
MANTKYNEEFLLYLAGFVDSDGSIIAQIKPNQSCKFKHALSLTFQVTQKTQRRWLLDKLVDEIGVGYVRDSGSVSHYYLSKIKPLHNFLTQLQPFLELKQKQANLVLKIIEQLPSAKESPDKFLEVCTWVDQVAALNDSKTRKTTSETVRAVLDSLSEKKKSSPAAGDSSVSNSEHIAPLSLPSSPPSVGSNKKFLLYLAGFVDGDGSIVAQIKPNQKYKFKHQLSLTFRVTQKTQRRWFLDKLVERIGAGHVYDFGSVSYYNLCEIKPLHNFLTQLQPFLKLKQKQANLVLKITEQLPSAKESPDKFLEVCTWVDQVAALNDSKTRKTTSETVRAVLDSLSEKKKSSP

SEQ ID NO: 122 - protein, 354 aa, artificial sequence
  SCOH-DMD37-pCLS4612
MANTKYNEEFLLYLAGFVDSDGSIIAQIKPTQSYKFKHQLRLTFQVTQKTQRRWFLDKLVDEIGVGYVRDSGSVSHYYLSEIKPLHNFLTQLQPFLELKQKQANLALKIIEQLPSAKESPDKFLEVCTWVDQIAALNDSKTRKTTSETVRAVLDSLSEKKKSSPAAGGSDKYNQALSKYNQALSKYNQALSGGGGSNKKFLLYLAGFVDGDGSIIAQIKPGQSYKFKHRLSLTFTVTQKTQRRWFLDKLVDRIGVGYVRDQGSVSYYILSEIKPLHNFLTQLQPFLRLKQKQANLVLKIIEQLPSAKESPDKFLEVCTWVDQIAALNDSKTRKTTSETVRAVLDSLSEKKKSSP

SEQ ID NO: 123 - protein, 354 aa, artificial sequence
  SCOH-DMD37-pCLS4613
MANTKYNEEFLLYLAGFVDSDGSIIAQIKPTQSYKFKHQLRLTFQVTQKTQRRWFLDKLVDEIGVGYVRDSGSVSHYYLSEIKPLHNFLTQLQPFLELKQKQANLALKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDGDGSIIAQIKPGQSYKFKHRLSLTFTVTQKTQRRWFLDKLVDRIGVGYVRDQGSVSYYILSEIKPLHNFLTQLQPFLRLKQKQANLVLKIIEQLPSAKESPDKFLEVCTWVDQVAALNDSKTRKTTSETVRAVLDSLSEKKKSSP

SEQ ID NO: 124 - protein, 354 aa, artificial sequence
  SCOH-DMD37-pCLS4614
MANTKYNEEFLLYLAGFVDSDGSIIAQIKPTQSYKFKHQLRLTFQVTQKTQRRWFLDKLVDEIGVGYVRDSGSVSHYYLSEIKPLHNFLTQLQPFLELKQKQANLALKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDGDGSIIAQIKPGQSYKFKHRLSLTFTVTQKTQRRWFLDKLVDRIGVGYVRDQGSVSYYILSKIKPLHNFLTQLQPFLRLKQKQANLVLKIIEQLPSAKESPDKFLEVCTWVDQVAALNDSKTRKTTSETVRAVLDSLSEKKKSSP

SEQ ID NO: 125 - protein, 349 aa, artificial sequence
  SCOH-DMD37-pCLS6602
MANTKYNEEFLLYLAGLVDGDGSIIAQIKPTQSYKFKHQLRLTFQVTQKTQRRWLLDKLVDEIGVGYVRDSGSVSHYYLSEIKPLHNFLTQLQPFLELKQKQANLVLKIIEQLPSAKESPDKFLEVCTWVDQVAALNDSKTRKTTSETVRAVLDSLSEKKKSSPAAGDSSVSNSEHIAPLSLPSSPPSVGSNKKFLLYLAGFVDSDGSIIAQIKPGQSYKFKHRLSLTFTVTQKTQRRWFLDKLVDRIGVGYVRDQGSVSYYILSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 126 - protein, 349 aa, artificial sequence
  SCOH-DMD37-pCLS6603
MANTKYNEEFLLYLAGFVDGDGSIIAQIKPTQSTKFKHQLSLTFQVTQKTQRRWFLDKLVDEIGVGYVADSGSVSNYRLSKIKPLHNFLTQLQPFLELKQKQANLVLKIIEQLPSAKESPDKFLEVCTWVDQVAALNDSKTRKTTSETVRAVLDSLSEKKKSSPAAGDSSVSNSEHIAPLSLPSSPPSVGSNKKFLLYLAGFVDSDGSIIAQIKPGQSYKFKHRLSLTFTVTQKTQRRWFLDKLVDRIGVGYVRDQGSVSYYILSEIKPLHNFLTQLQPFLRLKQKQANLVLKIIEQLPSAKESPDKFLEVCTWVDQVAALNDSKTRKTTSETVRAVLDSLSEKKKSSP

SEQ ID NO: 127 - protein, 354 aa, artificial sequence
  SCOH-DMD37-pCLS7389
MANTKYNEEFLLYLAGLVDGDGSIIAQIKPTQSYKFKHQLRLTFQVTQKTQRRWLLDKLVDEIGVGYVRDSGSVSHYYLSEIKPLHNFLTQLQPFLELKQKQANLALKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPGQSYKFKHRLSLTFTVTQKTQRRWFLDKLVDRIGVGYVRDQGSVSYYILSEIKPLHNFLTQLQPFLRLKQKQANLVLKIIEQLPSAKESPDKFLEVCTWVDQVAALNDSKTRKTTSETVRAVLDSLSEKKKSSP

SEQ ID NO: 128 - protein, 354 aa, artificial sequence
  SCOH-DMD37-pCLS7390
MANTKYNEEFLLYLAGLVDGDGSIIAQIKPTQSYKFKHQLRLTFQVTQKTQRRWLLDKLVDEIGVGYVRDSGSVSHYYLSKIKPLHNFLTQLQPFLELKQKQANLALKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPGQSYKFKHRLSLTFTVTQKTQRRWFLDKLVDRIGVGYVRDQGSVSYYILSEIKPLHNFLTQLQPFLRLKQKQANLVLKIIEQLPSAKESPDKFLEVCTWVDQVAALNDSKTRKTTSETVRAVLDSLSEKKKSSP

SEQ ID NO: 129 - protein, 354 aa, artificial sequence
  SCOH-DMD37-pCLS7391
MANTKYNEEFLLYLAGFVDGDGSIIAQIKPTQSYKFKHQLRLTFQVTQKTQRRWFLDKLVDEIGVGYVRDSGSVSHYYLSEIKPLHNFLTQLQPFLELKQKQANLALKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPGQSYKFKHRLSLTFTVTQKTQRRWFLDKLVDRIGVGYVRDQGSVSYYILSEIKPLHNFLTQLQPFLRLKQKQANLVLKIIEQLPSAKESPDKFLEVCTWVDQVAALNDSKTRKTTSETVRAVLDSLSEKKKSSP

SEQ ID NO: 130 - protein, 354 aa, artificial sequence
  SCOH-DMD37-pCLS7392
MANTKYNEEFLLYLAGFVDGDGSIIAQIKPTQSYKFKHQLRLTFQVTQKTQRRWFLDKLVDEIGVGYVRDSGSVSHYYLSEIKPLHNFLTQLQPFLELKQKQANLALKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPGQSYKFKHRLSLTFTVTQKTQRRWFLDKLVDRIGVGYVRDQGSVSYYILSKIKPLHNFLTQLQPFLRLKQKQANLVLKIIEQLPSAKESPDKFLEVCTWVDQVAALNDSKTRKTTSETVRAVLDSLSEKKKSSP

SEQ ID NO: 131 - DNA, 34 bp, Artificial
  Adaptor A
ccatctcatccctgcgtgtctccgactcagnnnn

SEQ ID NO: 132 - DNA, 30 bp, Artificial
  Adaptor B
cctatcccctgtgtgccttggcagtctcag

SEQ ID NO: 133 - DNA, 60 bp, Artificial
  DMD21_F primer
ccatctcatccctgcgtgtctccgactcagnnnnaatttctagaactacactaaaaaagc

SEQ ID NO: 134 - DNA, 57 bp, Artificial
  DMD21_R primer
cctatcccctgtgtgccttggcagtctcagaaacaacaagtacagtcttcattttgg

SEQ ID NO: 135 - DNA, 56 bp, Artificial
  DMD37_F primer
ccatctcatccctgcgtgtctccgactcagnnnntcaactgttgcctccggttctg

SEQ ID NO: 136 - DNA, 50 bp, Artificial
  DMD37_R primer
cctatcccctgtgtgccttggcagtctcagtgatgggtgctgaagtggca

SEQ ID NO: 137 - DNA, 29 bp, Artificial
  Integration matrix heterologous fragment
aattgcggccgcggtccggcgcgccttaa

SEQ ID NO: 138 - DNA, 27 bp, Artificial
  DMD21_KI_F PCR primer
aggcctccattcctttgaaggaattgg

SEQ ID NO: 139 - DNA, 22 bp, Artificial
  DMD21_KI_R PCR primer
ccggcgcgccttaaacttgagg

SEQ ID NO: 140 - DNA, 22 bp, Artificial
  DMD37_KI_F PCR primer
ttaaggcgcgccggaccgcggc

SEQ ID NO: 141 - DNA, 25 bp, Artificial
  DMD37_KI_R PCR primer
gcatcagttgcctggtatgtctagc

(19) United States
(12) Patent Application Publication
(10) Pub. No.: US 2013/0145487 A1
(43) Pub. Date: Jun. 6, 2013

(54) MEGANUCLEASE VARIANTS CLEAVING A DNA TARGET SEQUENCE FROM THE DYSTROPHIN GENE AND USES THEREOF

(71) Applicant: Frédéric Cedrone, Paris (FR)

(72) Inventor: Frédéric Cedrone, Paris (FR)

(73) Assignee: CELLECTIS, Paris (FR)

(21) Appl. No.: 13/697,623

(22) PCT Filed: May 12, 2011
(86) PCT No.: PCT/IB2011/001406

§ 371 Date: Jan. 31, 2013

Related U.S. Application Data
(60) Provisional application No. 61/333,987, filed on May 12, 2010.

(51) Int. Cl. C12N 9/16

(52) U.S. Cl. CPC C12N 9/16
USPC 800/13; 435/196; 536/23.2; 435/320.1; 435/252.3; 800/298; 424/94.6; 435/440; 800/21; 435/325; 435/419; 435/254.2

(57) ABSTRACT

The invention relates to meganuclease variants which cleave a DNA target sequence from the human dystrophin gene (DMD), to vectors encoding such variants, to a cell, an animal or a plant modified by such vectors and to the use of these meganuclease variants and products derived therefrom for genome therapy, ex vivo (gene cell therapy) and genome engineering including therapeutic applications and cell line engineering. The invention also relates to the use of meganuclease variants for inserting therapeutic transgenes other than DMD at the dystrophin gene locus, using this locus as a safe harbor locus. The invention also relates to the use of meganuclease variants for using the dystrophin gene locus as a landing pad to insert and express genes of interest.

### BACKGROUND OF THE INVENTION

1. Field of the Invention

The invention relates to meganuclease variants which cleave a DNA target sequence from the human Dystrophin gene (DMD) to vectors encoding such variants, to a cell, an animal or a plant modified by such vectors and to the use of these meganuclease variants and products derived therefrom for genome therapy, ex vivo (gene cell therapy) and genome engineering including therapeutic applications and cell line engineering.

2. Discussion of the Background Art

Duchenne Muscular Dystrophy is one of the most prevalent types of muscular dystrophy occurring for about 1/3500 boys worldwide. Duchenne Muscular Dystrophy is an X-linked recessive disorder caused by mutations in the dystrophin gene. The dystrophin gene is the largest known gene spanning ˜2.2 Mb at Xp21.1-21.2 encoding a major 14-kb mRNA transcript processed from 79 exons. The coding sequence amounts for less then 1% of the locus, the rest being the introns with the average size of 27 kb (the smallest is intron 14 which is only 107 bp and the largest is intron 44, spanning 248,401 bp). Duchenne muscular dystrophy is caused by a deficiency of a full-length 3685 amino acids (427 kD) dystrophin protein. The full length dystrophin expressed in skeletal muscle fibres, cardiomyocytes and smooth muscle cells contains 79 exons. Most of the mutations result in the absence of protein in the whole skeletal musculature and the cardiac muscle leading to a severe Duchenne phenotype characterized by a rapid progression of muscle degeneration.

There are currently several therapeutic avenues being pursued for Duchenne Muscular Dystrophy. (1) In vivo gene therapy with adeno-associated virus (AAV) vectors (Ohshima S et al, Liu M et al, Lai Y et al, Wang Z et al, Odom G L et al) using a μ-dystrophin to protect the muscle fibers (Harper S Q et al). The main drawbacks are that the μ-dystrophin gene may not fully replace the full length dystrophin in humans, the potential immune response against the AAV capsids and risks of random integration. (2) Transplantation of muscle precursor cells to introduce in muscle fibers normal nuclei containing the normal dystrophin gene (Peault B et al, Deasy B M et al, Ikemoto M et al, Sampaolesi M et al,). We have demonstrated that this restored the expression of dystrophin in up to 34% of the muscle fibers (Skuk D et al, 2006; Skuk D et al, 2007). This strategy requires multiple injections due to inefficient migration of myoblasts and immunosuppression to prevent rejection. (3) Pharmacologic rescue of a nonsense dystrophin mutation using PTC124, a potential approach for 13-15% of DMD patients, would require a life long administration of the drug (Welch E M et al, Wilton S et al). (4) Exon skipping aims to restore the translation of carboxy-terminal expression in patients with an out of frame deletion or a nonsense mutation by bypassing one or several exons (Williams J H et al, Jearawiriyapaisarn N et al, Yokota T et al). This will convert DMD patients into Becker-type patients. Its drawbacks are the requirement for a life-long administration of the exon skipping oligos and the potential long-term toxicity of these non-degradable oligonucleotides. Thus, there is still a need today for methods to address Duchenne Muscular Dystrophy.

The successful treatment of several X-SCID patients by gene therapy nearly 10 years ago was one of the most significant milestones in the field of gene therapy (Gaspar, H. B. et al Cavazzana-Calvo, M. et al.). This tremendous achievement was followed by significant success in other clinical trials addressing different diseases, including another form of SCID (Aiuti, A. et al.), Epidermolysis Bullosa (De Luca, M. et al) and Leber Amaurosis (Bainbridge, J. W. et al., Maguire, A. M. et al.). However, these initial successes have long been overshadowed by a series of severe adverse events (SAEs), i.e., the appearance of leukemia in X-SCID treated patients (Hacein-Bey-Abina, S. et al. 2003, Hacein-Bey-Abina, S. et al. 2008, Howe, S. J. et al.). All cases of leukemia, except one, could eventually be treated by chemotherapy and the approach appears globally as a success, but these SAEs highlighted the major risks of current gene therapy approaches.

Indeed, most of the gene therapy protocols that are being developed these days for the treatment of inherited diseases are based on the complementation of a mutant allele by an additional and functional copy of the disease-causing gene. In non-dividing tissues, such as retina, this copy can be borne by a non integrative vector, derived for example, from an Adeno Associated Virus (AAV) (Bainbridge, J. W. et al., Maguire, A. M. et al.). However, when targeting stem cells, such as hematopoietic stem cells (HSCs), whose fate is to proliferate, persistent expression becomes an issue, and there is a need for integrative vectors. Gamma-retroviral and lentiviral vectors, which integrate in the genome and replicate with the hosts' chromosomes, have proved efficient for this purpose (Chang, A. H. et al), but the random nature of their insertion has raised various concerns, all linked with gene expression. The cases of leukemia observed in the X-SCID trials were clearly linked to the activation of proto-oncogenes in the vicinity of the integration sites (Hacein-Bey-Abina, S. et al. 2003, Hacein-Bey-Abina, S. et al. 2008, Howe, S. J. et al.). In addition, inappropriate expression of the transgene could result in metabolic or immunological problems. Finally, insertion could result in the knock-out of endogenous genes. Gene expression concerns are also related to efficacy. For example, achieving a therapeutic level of expression of a beta-globin transgene proved to be a nightmare for a generation of researchers (May, C. et al., Sadelain, M. et al.).

Furthermore, even highly expressed transgenes can be silenced over time, and gene extinction remains a significant problem in the field (Ellis, J. et al.).

Therefore, there is a need in the art for a tool allowing the targeted insertion of transgenes into loci of the genome that can be considered as “safe harbors” for gene addition. In addition, it would be extremely advantageous if this tool could be used for inserting transgenes irrespective to their sequences, thereby allowing the treatment of numerous diseases by gene therapy using a same tool. Moreover, it would be extremely advantageous if this this tool allowed inserting transgenes with a high efficacity.

Several strategies have been developed to address these different issues. For example, new generations of safer viral vectors, like the Self Inactivating (SIN) gamma-retroviral and lentiviral vectors, should alleviate the activation of nearby potential oncogenes by the viral LTRs (Wilton S et al, Williams J H et al, Jearawiriyapaisarn N et al). In addition, vectors with restricted tropism or gene expression (Ellis, J. et al., Yu, S. F. et al., Yee, J. K. et al.) should help in avoiding inappropriate expression. However, several recent developments have highlighted the potential of other strategies, with the aim to achieve better control of the genomic events themselves. The use of meganuclease to induce high-frequency gene targeting is one of these methods.

Meganucleases can induce double-strand breaks (DSB) at specific unique sites in living cells, thereby enhancing gene targeting by 1000-fold or more in the vicinity of the cleavage site (Puchta et al., Nucleic Acids Res., 1993, 21, 5034-5040; Rouet et al., Mol. Cell. Biol., 1994, 14, 8096-8106; Choulika et al., Mol. Cell. Biol., 1995, 15, 1968-1973; Puchta et al., Proc. Natl. Acad. Sci. U.S.A., 1996, 93, 5055-5060; Sargent et al., Mol. Cell. Biol., 1997, 17, 267-277; Cohen-Tannoudji et al., Mol. Cell. Biol., 1998, 18, 1444-1448; Donoho, et al., Mol. Cell. Biol., 1998, 18, 4070-4078; Elliott et al., Mol. Cell. Biol., 1998, 18, 93-101).

Although several hundred natural meganucleases, also referred to as “homing endonucleases” have been identified (Chevalier, B. S. and B. L. Stoddard, Nucleic Acids Res., 2001, 29, 3757-3774), the repertoire of cleavable target sequences is too limited to allow the specific cleavage of a target site in a gene of interest as there is usually no cleavable site in a chosen gene of interest.

Theoretically, the making of artificial sequence-specific endonucleases with chosen specificities could alleviate this limit. To overcome this limitation, an approach adopted by a number of workers in this field is the fusion of Zinc-Finger Proteins (ZFPs) with the catalytic domain of Fokl, a class IIS restriction endonuclease, so as to make functional sequence-specific endonucleases (Smith et al., Nucleic Acids Res., 1999, 27, 674-681; Bibikova et al., Mol. Cell. Biol., 2001, 21, 289-297; Bibikova et al., Genetics, 2002, 161, 1169-1175; Bibikova et al., Science, 2003, 300, 764; Porteus, M. H. and D. Baltimore, Science, 2003, 300, 763-; Alwin et al., Mol. Ther., 2005, 12, 610-617; Urnov et al., Nature, 2005, 435, 646-651; Porteus, M. H., Mol. Ther., 2006, 13, 438-446). Such ZFP nucleases have been used for the engineering of the IL2RG gene in human lymphoid cells (Urnov et al., Nature, 2005, 435, 646-651).

The binding specificity of Cys2-His2 type Zinc-Finger Proteins, is easy to manipulate because specificity is driven by essentially four residues per zinc finger (Pabo et al., Annu. Rev. Biochem., 2001, 70, 313-340; Jamieson et al., Nat. Rev. Drug Discov., 2003, 2, 361-368). Studies from the Pabo laboratories have resulted in a large repertoire of novel artificial ZFPs, able to bind most G/ANNG/ANNG/ANN sequences (Rebar, E. J. and C. O. Pabo, Science, 1994, 263, 671-673; Kim, J. S. and C. O. Pabo, Proc. Natl. Acad. Sci. USA, 1998, 95, 2812-2817), Klug (Choo, Y. and A. Klug, Proc. Natl. Acad. Sci. USA, 1994, 91, 11163-11167; Isalan M. and A. Klug, Nat. Biotechnol., 2001, 19, 656-660) and Barbas (Choo, Y. and A. Klug, Proc. Natl. Acad. Sci. USA, 1994, 91, 11163-11167; Isalan M. and A. Klug, Nat. Biotechnol., 2001, 19, 656-660).

Nevertheless, ZFPs have serious limitations, especially for applications requiring a very high level of specificity, such as therapeutic applications. It was shown that FokI nuclease activity in ZFP fusion proteins can act with either one recognition site or with two sites separated by variable distances via a DNA loop (Catto et al., Nucleic Acids Res., 2006, 34, 1711-1720). Thus, the specificities of these ZFP nucleases are degenerate, as illustrated by high levels of toxicity in mammalian cells and Drosophila (Bibikova et al., Genetics, 2002, 161, 1169-1175; Bibikova et al., Science, 2003, 300, 764-).

To bypass these problems heretofore existing in the art, the inventors have adopted a different approach using engineered meganucleases.

In the wild, meganucleases are essentially represented by homing endonucleases. Homing Endonucleases (HEs) are a widespread family of natural meganucleases including hundreds of proteins families (Chevalier, B. S. and B. L. Stoddard, Nucleic Acids Res., 2001, 29, 3757-3774). These proteins are encoded by mobile genetic elements which propagate by a process called “homing”: the endonuclease cleaves a cognate allele from which the mobile element is absent, thereby stimulating a homologous recombination event that duplicates the mobile DNA into the recipient locus. Given their exceptional cleavage properties in terms of efficacy and specificity, they could represent ideal scaffold to derive novel, highly specific endonucleases.

HEs belong to four major families. The LAGLIDADG family, named after a conserved peptidic motif involved in the catalytic center, is the most widespread and the best characterized group. Seven structures are now available. Whereas most proteins from this family are monomeric and display two LAGLIDADG motifs, a few have only one motif, but dimerize to cleave palindromic or pseudo-palindromic target sequences.

Although the LAGLIDADG peptide is the only conserved region among members of the family, these proteins share a very similar architecture (FIG. 2A). The catalytic core is flanked by two DNA-binding domains with a perfect two-fold symmetry for homodimers such as I-CreI (Chevalier, et al., Nat. Struct. Biol., 2001, 8, 312-316) and I-MsoI (Chevalier et al., J. Mol. Biol., 2003, 329, 253-269) and with a pseudo symmetry for monomers such as I-SceI (Moure et al., J. Mol. Biol., 2003, 334, 685-69, I-DmoI (Silva et al., J. Mol. Biol., 1999, 286, 1123-1136) or I-AniI (Bolduc et al., Genes Dev., 2003, 17, 2875-2888). Both monomers or both domains of monomeric proteins contribute to the catalytic core, organized around divalent cations. Just above the catalytic core, the two LAGLIDADG peptides play also an essential role in the dimerization interface. DNA binding depends on two typical saddle-shaped αββαββα folds, sitting on the DNA major groove. Other domains can be found, for example in inteins such as PI-PfuI (Ichiyanagi et al., J. Mol. Biol., 2000, 300, 889-901) and PI-SceI (Moure et al., Nat. Struct. Biol., 2002, 9, 764-770), which protein splicing domain is also involved in DNA binding.

The making of functional chimeric meganucleases, by fusing the N-terminal I-DmoI domain with an I-CreI monomer (Chevalier et al., Mol. Cell., 2002, 10, 895-905; Epinat et al., Nucleic Acids Res, 2003, 31, 2952-62; International PCT Applications WO 03/078619 and WO 2004/031346) have demonstrated the plasticity of meganucleases.

Different groups have used a semi-rational approach to locally alter the specificity of I-CreI (Seligman et al., Genetics, 1997, 147, 1653-1664; Sussman et al., J. Mol. Biol., 2004, 342, 31-41; International PCT Applications WO 2006/097784 and WO 2006/097853; Arnould et al., J. Mol. Biol., 2006, 355, 443-458; Rosen et al., Nucleic Acids Res., 2006, 34, 4791-4800; Smith et al., Nucleic Acids Res., 2006, 34, e149), I-SceI (Doyon et al., J. Am. Chem. Soc., 2006, 128, 2477-2484), PI-SceI (Gimble et al., J. Mol. Biol., 2003, 334, 993-1008) and I-MsoI (Ashworth et al., Nature, 2006, 441, 656-659).

In addition, hundreds of I-CreI derivatives with locally altered specificity were engineered by combining the semi-rational approach and High Throughput Screening:
Residues Q44, R68 and R70 or Q44, R68, D75 and 177 of I-CreI were mutagenized and a collection of variants with altered specificity at positions ±3 to 5 of the DNA target (5NNN DNA target) were identified by screening (International PCT Applications WO 2006/097784 and WO 2006/097853; Arnould et al., J. Mol. Biol., 2006, 355, 443-458; Smith et al., Nucleic Acids Res., 2006, 34, e149).
Residues K28, N30 and Q38 or N30, Y33, and Q38 or K28, Y33, Q38 and S40 of 1-CreI were mutagenized and a collection of variants with altered specificity at positions ±8 to 10 of the DNA target (10 NNN DNA target) were identified by screening (Smith et al., Nucleic Acids Res., 2006, 34, e149; International PCT Applications WO 2007/060495 and WO 2007/049156).

Two different variants were combined and assembled in a functional heterodimeric endonuclease able to cleave a chimeric target resulting from the fusion of a different half of each variant DNA target sequence (Arnould et al., precited; International PCT Applications WO 2006/097854 and WO 2007/034262), as illustrated on FIG. 2B. Interestingly, the novel proteins had kept proper folding and stability, high activity, and a narrow specificity.

Furthermore, residues 28 to 40 and 44 to 77 of I-CreI were shown to form two separable functional subdomains, able to bind distinct parts of a homing endonuclease half-site (Smith et al. Nucleic Acids Res., 2006, 34, e149; International PCT Applications WO 2007/049095 and WO 2007/057781).

The combination of mutations from the two subdomains of I-CreI within the same monomer allowed the design of novel chimeric molecules (homodimers) able to cleave a palindromic combined DNA target sequence comprising the nucleotides at positions ±3 to 5 and ±8 to 10 which are bound by each subdomain (Smith et al., Nucleic Acids Res., 2006, 34, e149; International PCT Applications WO 2007/060495 and WO 2007/049156), as illustrated on FIG. 2C.

The combination of the two former steps allows a larger combinatorial approach, involving four different subdomains. The different subdomains can be modified separately and combined to obtain an entirely redesigned meganuclease variant (heterodimer or single-chain molecule) with chosen specificity, as illustrated on FIG. 2D. In a first step, couples of novel meganucleases are combined in new molecules (“half-meganucleases”) cleaving palindromic targets derived from the target one wants to cleave. Then, the combination of such “half-meganuclease” can result in a heterodimeric species cleaving the target of interest. The assembly of four sets of mutations into heterodimeric endonucleases cleaving a model target sequence or a sequence from different genes has been described in the following patent applications: XPC gene (WO2007093918), RAG gene (WO2008010093), HPRT gene (WO2008059382), beta-2 microglobulin gene (WO2008102274), Rosa26 gene (WO2008152523), Human hemoglobin beta gene (WO2009013622) and Human Interleukin-2 receptor gamma chain (WO2009019614).

These variants can be used to cleave genuine chromosomal sequences and have paved the way for novel perspectives in several fields, including gene therapy.

However, even though the base-pairs ±1 and ±2 do not display any contact with the protein, it has been shown that these positions are not devoid of content information (Chevalier et al., J. Mol. Biol., 2003, 329, 253-269), especially for the base-pair ±1 and could be a source of additional substrate specificity (Argast et al., J. Mol. Biol., 1998, 280, 345-353; Jurica et al., Mol. Cell., 1998, 2, 469-476; Chevalier, B. S. and B. L. Stoddard, Nucleic Acids Res., 2001, 29, 3757-3774). In vitro selection of cleavable I-CreI target (Argast et al., precited) randomly mutagenized, revealed the importance of these four base-pairs on protein binding and cleavage activity. It has been suggested that the network of ordered water molecules found in the active site was important for positioning the DNA target (Chevalier et al., Biochemistry, 2004, 43, 14015-14026). In addition, the extensive conformational changes that appear in this region upon I-CreI binding suggest that the four central nucleotides could contribute to the substrate specificity, possibly by sequence dependent conformational preferences (Chevalier et al., 2003, precited). As shown by Arnould et al. (Arnould et al. J Mol Biol 2007 371 49-65) in the XPC gene the inventors have now found active new endonucleases cleaving targets within the DMD gene containing changes in these four central nucleotides, which are G₋₂T₋₁A₊₁C₊₂ in the wildtype palindromic I-CreI target C1221 (SEQ ID NO: 2); these meganuclease variants and products derived therefrom could be used for genome therapy, ex vivo (gene cell therapy) and genome engineering including therapeutic applications and cell line engineering.

### SUMMARY OF THE INVENTION

Three different strategies can be envisioned with meganucleases, in order to correct a genetic defect.

First approach is the correction of the mutated gene itself. This gene correction strategy requires very precise genome editing at the targeted locus (FIG. 1-B). The advantage being, that it directly addresses the cause of the disease: instead of compensating the effect of the mutation by a second genome alteration (such as an insertion in a safe harbor), the true reversion of the disease-causing mutation is the least invasive event one can imagine. However, this precision comes with an inherent drawback: the correction of the mutation, usually based on homologous gene repair, is a very local event, and one needs a different meganuclease for each disease, and in most cases, for each mutation or at least each mutation hotspot related to the disease. This kind of approach can be envisioned as a treatment for monogenic diseases in which a prevalent mutation is responsible for the majority of the cases, such as Sickle Cell Anemia (SCA), in which a single mutation (E6V) is present in 100% of the patients (Sadelain, M. et al) and Cystic Fibrosis FTR, where almost 70% of the patients carry a deletion of a Phenylalanine in position 508 (Rosenecker, J. et al) of the CFTR gene. However, it is much more difficult to envision for a large gene such as DMD, with the mutations scattered along a 2 Mb regions.

Another approach involves use of an intermediate approach between targeted gene correction and gene addition, named here “exon knock-in” (FIG. 1-C). In this approach, a complete or partial cDNA of the affected gene would be integrated in the very endogenous targeted locus. This genomic insertion would be less invasive to the cellular genome, since the locus itself would act as a kind of safe harbor for the specific disease. However, this does not alleviate all the possible risks: the resulting gene could lack sequences involved in gene regulation if they are found in the missing introns. Additionally, the genomic locus would be significantly modified, with potential consequences at the transcriptional level. In a more refined form, gene replacement could be used to replace a whole region of the locus.

A promising alternative to random integration of viral vectors is a site-specific integration in a safe locus (FIG. 1-A). The major challenge is the availability of a region in the genome that could be considered as a “safe harbor” for gene addition. This locus should be chosen in a way that the probability of insertional mutagenesis would be minimized, retaining a long-term and high level of expression of the transgene.

Given the large size of the DMD gene and the large diversity of mutations resulting in Duchenne's Muscular Dystrophy, among which, a variety of deletions and duplications, the exon KI strategy is the most adapted to correct this gene in a large number of cases. Therefore, a first main aspect of the present invention concerns endonucleases variants that could be used in this approach to induce a double strand break in the DMD gene and for genome therapy of DMD disease and also allowing further experimental study of this important disease in cellular or other types of model systems.

The “exon knock-in” approach has the advantage of allowing the use of a same reagent to correct many different mutations, and treat many different patients. Eventually, targeting a “safe harbor” would allow to treat different diseases using a same reagent (although one would also have to use different inserts). It has therefore several advantages over the other approaches. However, its feasibility depends on the identification of a good “safe harbor” locus, which should display the following properties (i) it should allow for stable and sufficient expression of the inserted transgene, in order to insure efficacy of the treatment (ii) insertion in this locus should have no impact on the expression of other genes.

Given the very large size of the DMD locus, it is unlikely that targeted insertion into this locus could result into cis-activation of other genes. However, it could disrupt the DMD gene itself. Therefore, one can consider the DMD locus as a safe harbor:
(i) in cells that do not normally express DMD, provided the insert can be expressed from this locus.
(ii) in cells that do normally express DMD, provided the insertion does not affect the expression of DMD, or provided there remain a functional allele in the cell. For example, insertion in introns can be made with no or minor modification of the expression pattern.

Therefore, in a second main aspect of the present invention, the inventors have found that endonucleases variants targeting DMD gene can be used for inserting therapeutic transgenes other than DMD at the dystrophin gene locus, using this locus as a safe harbor locus.

In a third main aspect of the present invention, the inventors have found that the dystrophin locus could be used as a landing pad to insert and express genes of interest (GOIs).

The above objects highlight certain aspects of the invention. Additional objects, aspects and embodiments of the invention are found in the following detailed description of the invention.

### BRIEF DESCRIPTION OF THE FIGURES

In addition to the preceding features, the invention further comprises other features which will emerge from the description which follows, which refers to examples illustrating the I-CreI meganuclease variants and their uses according to the invention, as well as to the appended drawings. A more complete appreciation of the invention and many of the attendant advantages thereof will be readily obtained as the same becomes better understood by reference to the following Figures in conjunction with the detailed description below.

FIG. 1: Illustration of three different strategies for correcting a genetic defect with meganuclease-induced recombination. A. Site-specific integration in a safe locus; the major challenge is the availability of such a region in the genome that could be considered as a “safe harbor” for gene addition. This locus should be chosen in a way that the probability of insertional mutagenesis would be minimized, retaining a long-term and high level of expression of the transgene. B. Gene correction. A mutation occurs within the dystrophin gene. Upon cleavage by a meganuclease and recombination with a repair matrix the deleterious mutation is corrected. C. Exonic sequences knock-in. A mutation occurs within the dystrophin gene. The mutated mRNA transcript is featured below the gene. In the repair matrix, all exons necessary to reconstitute a complete cDNA are fused in frame, with a polyadenylation site to stop transcription in 3′. Introns and exons sequences can be used as homologous regions. Exonic sequences knock-in results into an engineered gene, transcribed into a mRNA able to code for a functional dystrophin protein.

FIG. 2: Modular structure of homing endonucleases and the combinatorial approach for custom meganucleases design. A. Tridimensional structure of the I-CreI homing endonuclease bound to its DNA target. The catalytic core is surrounded by two αββαββα folds forming a saddle-shaped interaction interface above the DNA major groove. B. Different binding sequences derived from the I-CreI target sequence (top right and bottom left) to obtain heterodimers or single chain fusion molecules cleaving non palindromic chimeric targets (bottom right). C. The identification of smaller independent subunit, i.e., subunit within a single monomer or αββαββα fold (top right and bottom left) would allow for the design of novel chimeric molecules (bottom right), by combination of mutations within a same monomer. Such molecules would cleave palindromic chimeric targets (bottom right). D. The combination of the two former steps would allow a larger combinatorial approach, involving four different subdomains. In a first step, couples of novel meganucleases could be combined in new molecules (“half-meganucleases”) cleaving palindromic targets derived from the target one wants to cleave. Then, the combination of such “half-meganuclease” can result in an heterodimeric species cleaving the target of interest. Thus, the identification of a small number of new cleavers for each subdomain would allow for the design of a very large number of novel endonucleases.

FIG. 3: Exon Knock in strategies by insertion (A) or by replacement (B) for the dystrophin gene.

FIG. 4: DMD21 and DMD21-derived targets. The DMD21 target sequence (SEQ ID NO: 4) and its derivatives 10AAC_P (SEQ ID NO: 5), 10TAC_P (SEQ ID NO: 7), 5CAA_P (SEQ ID NO: 6) and 5TTG_P (SEQ ID NO: 8), P stands for Palindromic) are derivatives of C1221, found to be cleaved by previously obtained I-CreI mutants. C1221 (SEQ ID NO: 2), 10AAC_P (SEQ ID NO: 5), 10TAC_P (SEQ ID NO: 7), 5CAA_P (SEQ ID NO: 6) and 5TTG_P (SEQ ID NO: 8) were first described as 24 bp sequences, but structural data suggest that only the 22 bp are relevant for protein/DNA interaction. DMD21 (SEQ ID NO: 4) is the DNA sequence located in the human dystrophin gene at position 993350-993373. DMD21.3 (SEQ ID NO: 9) is the palindromic sequence derived from the left part of DMD21, and DMD21.4 (SEQ ID NO: 10) is the palindromic sequence derived from the right part of DMD21.

FIG. 5: Activity cleavage in CHO cells of single chain heterodimer SCOH-DMD21: pCLS2872, pCLS2873, pCLS2874, pCLS2875, pCLS3385, pCLS3387 and pCLS3388 compared to ISceI (pCLS1090) and SCOH-RAG-CLS (pCLS2222) meganucleases as positive controls. The empty vector control (pCLS1069) has also been tested on each target. Plasmid pCLS1728 contains control RAG1.10.1 target sequence.

FIG. 5 bis: Activity cleavage in CHO cells of single chain heterodimer SCOH-DMD21: pCLS2874, pCLS5353, pCLS5354, pCLS5355 and pCLS5356 compared to ISceI and SCOH-RAG meganucleases as positive controls.

FIG. 6: DMD24 and DMD24-derived targets. The DMD24 target sequence (SEQ ID NO: 11) and its derivatives 10TAC_P (SEQ ID NO: 12), 10TAT_P (SEQ ID NO: 14), 5ATT_P (SEQ ID NO: 13) and 5GAC_P ((SEQ ID NO: 15), P stands for Palindromic) are derivatives of C1221, found to be cleaved by previously obtained I-CreI mutants. C1221 (SEQ ID NO: 2), 10TAC_P (SEQ ID NO: 12), 10TAT_P (SEQ ID NO: 14), 5ATT_P (SEQ ID NO: 13) and 5GAC_P ((SEQ ID NO: 15) were first described as 24 bp sequences, but structural data suggest that only the 22 bp are relevant for protein/DNA interaction. DMD24 (SEQ ID NO: 11) is the DNA sequence located in the human dystrophin gene at position 995930-995953. DMD24.2 (SEQ ID NO: 16) differs from DMD24 at positions −2; −1; +1; +2 where I-CreI cleavage site (GTAC) substitutes the corresponding DMD24 sequence. DMD24.3 (SEQ ID NO: 17) is the palindromic sequence derived from the left part of DMD24.2, and DMD24.4 (SEQ ID NO: 18) is the palindromic sequence derived from the right part of DMD24.2. DMD24.5 (SEQ ID NO: 19) is the palindromic sequence derived from the left part of DMD24, and DMD24.6 (SEQ ID NO: 20) is the palindromic sequence derived from the right part of DMD24.

FIG. 7: Activity cleavage in CHO cells of single chain heterodimer SCOH-DMD24 pCLS3402 compared to ISceI (pCLS1090) and SCOH-RAG-CLS (pCLS2222) meganucleases as positive controls. The empty vector control (pCLS1069) has also been tested on each target. Plasmid pCLS1728 contains control RAG1.10.1 target sequence.

FIG. 8: DMD31 and DMD31-derived targets. The DMD31 target sequence (SEQ ID NO: 21) and its derivatives 10TGT_P (SEQ ID NO: 22), 10AAC_P (SEQ ID NO: 24), 5GAT_P (SEQ ID NO: 23) and 5ATT_P (SEQ ID NO: 25), (P stands for Palindromic) are derivatives of C1221, found to be cleaved by previously obtained I-CreI mutants. C1221 (SEQ ID NO: 2), 10TGT_P (SEQ ID NO: 22), 10AAC_P (SEQ ID NO: 24), 5GAT_P (SEQ ID NO: 23) and 5ATT_P (SEQ ID NO: 25) were first described as 24 bp sequences, but structural data suggest that only the 22 bp are relevant for protein/DNA interaction. DMD31 (SEQ ID NO: 21) is the DNA sequence located in the human dystrophin gene at position 1125314-1125337. DMD31.2 (SEQ ID NO: 26) differs from DMD31 at positions −2; −1; +1; +2 where I-CreI cleavage site (GTAC) substitutes the corresponding DMD31 sequence. DMD31.3 (SEQ ID NO: 27) is the palindromic sequence derived from the left part of DMD31.2, and DMD31.4 (SEQ ID NO: 28) is the palindromic sequence derived from the right part of DMD31.2. DMD31.5 (SEQ ID NO: 29) is the palindromic sequence derived from the left part of DMD31, and DMD31.6 (SEQ ID NO: 30) is the palindromic sequence derived from the right part of DMD31.

FIG. 9: Activity cleavage in CHO cells of single chain heterodimer SCOH-DMD31: pCLS3631-SCOH-DD31b12-B and pCLS3633-SCOH-DD31b12-D compared to ISceI (pCLS1090) and SCOH-RAG-CLS (pCLS2222) meganucleases as positive controls. The empty vector control (pCLS1069) has also been tested on each target. Plasmid pCLS1728 contains control RAG1.10.1 target sequence.

FIG. 10: DMD33 and DMD33-derived targets. The DMD33 target sequence (SEQ ID NO: 31) and its derivatives 10ATC_P (SEQ ID NO: 32), 10GAG_P (SEQ ID NO: 34), 5GCC_P (SEQ ID NO: 33) and 5ACT_P (SEQ ID NO: 35), (P stands for Palindromic) are derivatives of C1221, found to be cleaved by previously obtained I-CreI mutants. C1221 (SEQ ID NO: 2), 10ATC_P (SEQ ID NO: 32), 10GAG_P (SEQ ID NO: 34), 5GCC_P (SEQ ID NO: 33) and 5ACT_P (SEQ ID NO: 35) were first described as 24 bp sequences, but structural data suggest that only the 22 bp are relevant for protein/DNA interaction. DMD33 (SEQ ID NO: 31) is the DNA sequence located in the human dystrophin gene at position 1031834-1031857. DMD33.2 (SEQ ID NO: 36) differs from DMD33 at positions −2; −1; +1; +2 where I-CreI cleavage site (GTAC) substitutes the corresponding DMD33 sequence. DMD33.3 (SEQ ID NO: 37) is the palindromic sequence derived from the left part of DMD33.2, and DMD33.4 (SEQ ID NO: 38) is the palindromic sequence derived from the right part of DMD33.2. DMD33.5 (SEQ ID NO: 39) is the palindromic sequence derived from the left part of DMD33, and DMD33.6 (SEQ ID NO: 40) is the palindromic sequence derived from the right part of DMD33.

FIG. 11: Activity cleavage in CHO cells of single chain heterodimer SCOH-DMD33 pCLS3326 and pCLS3333 compared to ISceI (pCLS1090) and SCOH-RAG-CLS (pCLS2222) meganucleases as positive controls. The empty vector control (pCLS1069) has also been tested on each target. Plasmid pCLS1728 contains control RAG1.10.1 target sequence.

FIG. 12: DMD35 and DMD35-derived targets. The DMD35 target sequence (SEQ ID NO: 41) and its derivatives 10TTT_P (SEQ ID NO: 42), 10AAT_P (SEQ ID NO: 44), 5GTT_P (SEQ ID NO: 43) and 5ACT_P (SEQ ID NO: 45), (P stands for Palindromic) are derivatives of C1221, found to be cleaved by previously obtained I-CreI mutants. C1221 (SEQ ID NO: 2), 10TTT_P (SEQ ID NO: 42), 10AAT_P (SEQ ID NO: 44), 5GTT_P (SEQ ID NO: 43) and 5ACT_P (SEQ ID NO: 45) were first described as 24 bp sequences, but structural data suggest that only the 22 bp are relevant for protein/DNA interaction. DMD35 (SEQ ID NO: 41) is the DNA sequence located in the human dystrophin gene at position 1561221-1561244. DMD35.2 (SEQ ID NO: 46) differs from DMD35 at positions −2; −1; +1; +2 where I-CreI cleavage site (GTAC) substitutes the corresponding DMD35 sequence. DMD35.3 (SEQ ID NO: 47) is the palindromic sequence derived from the left part of DMD35.2, and DMD35.4 (SEQ ID NO: 48) is the palindromic sequence derived from the right part of DMD35.2. DMD35.5 (SEQ ID NO: 49) is the palindromic sequence derived from the left part of DMD35, and DMD35.6 (SEQ ID NO: 50) is the palindromic sequence derived from the right part of DMD35.

FIG. 13: DMD37 and DMD37-derived targets. The DMD37 target sequence (SEQ ID NO: 51) and its derivatives 10ATC_P (SEQ ID NO: 52), 10AGG_P (SEQ ID NO: 54), 5GTT_P (SEQ ID NO: 53) and 5GAT_P (SEQ ID NO: 55), (P stands for Palindromic) are derivatives of C1221, found to be cleaved by previously obtained I-CreI mutants. C1221 (SEQ ID NO: 2), 10ATC_P (SEQ ID NO: 52), 10AGG_P (SEQ ID NO: 54), 5GTT_P (SEQ ID NO: 53) and 5GAT_P (SEQ ID NO: 55) were first described as 24 bp sequences, but structural data suggest that only the 22 bp are relevant for protein/DNA interaction. DMD37 (SEQ ID NO: 51) is the DNA sequence located in the human dystrophin gene at position 1659873-1659896. DMD37.2 (SEQ ID NO: 56) differs from DMD37 at positions −2; −1; +1; +2 where I-CreI cleavage site (GTAC) substitutes the corresponding DMD37 sequence. DMD37.3 (SEQ ID NO: 57) is the palindromic sequence derived from the left part of DMD37.2, and DMD37.4 (SEQ ID NO: 58) is the palindromic sequence derived from the right part of DMD37.2. DMD37.5 (SEQ ID NO: 59) is the palindromic sequence derived from the left part of DMD37, and DMD37.6 (SEQ ID NO: 60) is the palindromic sequence derived from the right part of DMD37.

FIG. 14: Activity cleavage in CHO cells of single chain heterodimer SCOH-DMD37 pCLS4606, pCLS4607-SCOH-DMD37b11-B, pCLS4608-SCOH-DMD37b11-C, pCLS4609, pCLS4610, pCLS4611, pCLS4612, pCLS4613 and pCLS4614 compared to ISceI (pCLS1090) and SCOH-RAG-CLS (pCLS2222) meganucleases as positive controls. The empty vector control (pCLS1069) has also been tested on each target. Plasmid pCLS1728 contains control RAG1.10.1 target sequence.

FIG. 14 bis: Activity cleavage in CHO cells of single chain heterodimer SCOH-DMD37 pCLS4607-SCOH-DMD37b11-B, pCLS4608-SCOH-DMD37b11-C, pCLS4613 and pCLS4614, pCLS6602, pCLS6603, pCLS7389, pCLS7390, pCLS7391 and pCLS7392 compared to ISceI and SCOH-RAG-CLS meganucleases as positive controls. The empty vector control (pCLS1069) has also been tested on each target. Plasmid pCLS1728 contains control RAG1.10.1 target sequence (not shown).

FIG. 15: Vector Map of pCLS1072

FIG. 16: Vector Map of pCLS1090

FIG. 17: Vector Map of pCLS2222

FIG. 18: Vector Map of pCLS1853

FIG. 19: Vector Map of pCLS1107

FIG. 20: Vector Map of pCLS0002

FIG. 21: Vector Map of pCLS1069

FIG. 22: Vector Map of pCLS1058

FIG. 23: Vector Map of pCLS1728

FIG. 24: Vector Maps of pIM-DMD-Luc and pIM-DMD-MCS

FIG. 25: Description of universal integration matrices. Schematic representation of the different genetic elements introduced in universal integration matrices. First, positive and selection marker genes are added in two different places: the former inserted in and the latter inserted out of the recombinogenic element. Second, different restriction sites have been introduced: 8 bp cutting sites for the cloning of left and right homology arms for any type of integration locus, a multiple cloning site (MCS) for the integration of any GOI and other restriction sites in the case of additional element cloning (i.e. enhancers, silencers).

FIG. 26: Location of PCR primers F_HS2_PCRsc and R_HS2_PCRsc on pIM-DMD-Luc integration matrix.

FIG. 27: Southern blot analysis of human DMD targeted clones. Panel A: Hybridization of the neo probe on gDNA digested with EcoRV restriction enzyme from Neo^{R}PCR⁺ HEK293 clones; C—: Control lane (gDNA from native HEK293). Panel B: Hybridization of the neo probe on gDNA digested with EcoRV restriction enzyme from Neo^{R}PCR⁺ U 2-OS clones. Right arrows indicate the 4.8 kb expected band, demonstrating the correct targeted integration at the DMD locus.

FIG. 28: Luciferase reporter gene expression under the control of six different promoters in human DMD-targeted HEK293 clones.

FIG. 29: Activity cleavage in CHO cells of single chain heterodimer SCOH-DMD35 pCLS4901, pCLS4902, pCLS4903 and pCLS4904 compared to ISceI and SCOH-RAG-CLS meganucleases as positive controls. The empty vector control (pCLS1069) has also been tested on each target. Plasmid pCLS1728 contains control RAG1.10.1 target sequence (not shown).

FIG. 30: Activity cleavage in CHO cells of single chain heterodimer SCOH-DMD35 pCLS4902, pCLS4904 and pCLS6601 compared to ISceI and SCOH-RAG-CLS meganucleases as positive controls. The empty vector control (pCLS1069) has also been tested on each target. Plasmid pCLS1728 contains control RAG1.10.1 target sequence (not shown).

### DETAILED DESCRIPTION OF THE INVENTION

Unless specifically defined herein below, all technical and scientific terms used herein have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

According to a first aspect of the present invention is an I-CreI variant, which has two I-CreI monomers and at least one of the two I-CreI monomers has at least two substitutions, where there is at least one mutation in each of the two functional subdomains of the LAGLIDADG core domain situated from positions 26 to 40 and 44 to 77 of I-CreI, respectively, and said variant cleaves a DNA target sequence from the DMD gene. Within this embodiment, the I-CreI variant is obtained by a method comprising at least the steps of:

(a) constructing a first series of I-CreI variants having at least one substitution in a first functional subdomain of the LAGLIDADG core domain situated from positions 26 to 40 of I-CreI,

(b) constructing a second series of I-CreI variants having at least one substitution in a second functional subdomain of the LAGLIDADG core domain situated from positions 44 to 77 of I-CreI,

(c) selecting and/or screening the variants from the first series of step (a) which are able to cleave a mutant I-CreI site wherein at least one of (i) the nucleotide triplet in positions −10 to −8 of the I-CreI site has been replaced with the nucleotide triplet which is present in positions −10 to −8 of said DNA target sequence from DMD gene and (ii) the nucleotide triplet in positions +8 to +10 has been replaced with the reverse complementary sequence of the nucleotide triplet which is present in position −10 to −8 of said DNA target sequence from DMD gene,

(d) selecting and/or screening the variants from the second series of step (b) which are able to cleave a mutant I-CreI site wherein at least one of (i) the nucleotide triplet in positions −5 to −3 of the I-CreI site has been replaced with the nucleotide triplet which is present in positions −5 to −3 of said DNA target sequence from DMD gene and (ii) the nucleotide triplet in positions +3 to +5 has been replaced with the reverse complementary sequence of the nucleotide triplet which is present in position −5 to −3 of said DNA target sequence from DMD gene,

(e) selecting and/or screening the variants from the first series of step (a) which are able to cleave a mutant I-CreI site wherein at least one of (i) the nucleotide triplet in positions +8 to +10 of the I-CreI site has been replaced with the nucleotide triplet which is present in positions +8 to +10 of said DNA target sequence from DMD gene and (ii) the nucleotide triplet in positions −10 to −8 has been replaced with the reverse complementary sequence of the nucleotide triplet which is present in position +8 to +10 of said DNA target sequence from DMD gene,

(f) selecting and/or screening the variants from the second series of step (b) which are able to cleave a mutant I-CreI site wherein at least one of (i) the nucleotide triplet in positions +3 to +5 of the I-CreI site has been replaced with the nucleotide triplet which is present in positions +3 to +5 of said DNA target sequence from DMD gene and (ii) the nucleotide triplet in positions −5 to −3 has been replaced with the reverse complementary sequence of the nucleotide triplet which is present in position +3 to +5 of said DNA target sequence from DMD gene,

(g) combining in a single variant, the mutation(s) in positions 26 to 40 and 44 to 77 of two variants from step (c) and step (d), to obtain a novel homodimeric I-CreI variant which cleaves a sequence wherein (i) the nucleotide triplet in positions −10 to −8 is identical to the nucleotide triplet which is present in positions −10 to −8 of said DNA target sequence from DMD gene, (ii) the nucleotide triplet in positions +8 to +10 is identical to the reverse complementary sequence of the nucleotide triplet which is present in positions −10 to −8 of said DNA target sequence from DMD gene, (iii) the nucleotide triplet in positions −5 to −3 is identical to the nucleotide triplet which is present in positions −5 to −3 of said DNA target sequence from DMD gene and (iv) the nucleotide triplet in positions +3 to +5 is identical to the reverse complementary sequence of the nucleotide triplet which is present in positions −5 to −3 of said DNA target sequence from DMD gene, and/or

(h) combining in a single variant, the mutation(s) in positions 26 to 40 and 44 to 77 of two variants from step (e) and step (f), to obtain a novel homodimeric I-CreI variant which cleaves a sequence wherein (i) the nucleotide triplet in positions +8 to +10 of the I-CreI site has been replaced with the nucleotide triplet which is present in positions +8 to +10 of said DNA target sequence from DMD gene and (ii) the nucleotide triplet in positions −10 to −8 is identical to the reverse complementary sequence of the nucleotide triplet in positions +8 to +10 of said DNA target sequence from DMD gene, (iii) the nucleotide triplet in positions +3 to +5 is identical to the nucleotide triplet which is present in positions +3 to +5 of said DNA target sequence from DMD gene, (iv) the nucleotide triplet in positions −5 to −3 is identical to the reverse complementary sequence of the nucleotide triplet which is present in positions +3 to +5 of said DNA target sequence from DMD gene,

(i) combining the variants obtained in steps (g) and (h) to form heterodimers, and (j) selecting and/or screening the heterodimers from step (i) which cleave said DNA target sequence from DMD gene.

In the present patent application the terms meganuclease (s) and variant (s) and variant meganuclease (s) will be used interchangeably herein.

One of the step(s) (c), (d), (e), (0, (g), (h) or (i) may be omitted. For example, if step (c) is omitted, step (d) is performed with a mutant I-CreI target wherein both nucleotide triplets at positions −10 to −8 and −5 to −3 have been replaced with the nucleotide triplets which are present at positions −10 to −8 and −5 to −3, respectively of said genomic target, and the nucleotide triplets at positions +3 to +5 and +8 to +10 have been replaced with the reverse complementary sequence of the nucleotide triplets which are present at positions −5 to −3 and −10 to −8, respectively of said genomic target.

The (intramolecular) combination of mutations in steps (g) and (h) may be performed by amplifying overlapping fragments comprising each of the two subdomains, according to well-known overlapping PCR techniques.

The (intermolecular) combination of the variants in step (i) is performed by co-expressing one variant from step (g) with one variant from step (h), so as to allow the formation of heterodimers. For example, host cells may be modified by one or two recombinant expression vector(s) encoding said variant(s). The cells are then cultured under conditions allowing the expression of the variant(s), so that heterodimers are formed in the host cells, as described previously in the International PCT Application WO 2006/097854 and Arnould et al., J. Mol. Biol., 2006, 355, 443-458.

The selection and/or screening in steps (c), (d), (e), (0, and/or (j) may be performed by measuring the cleavage activity of the variant according to the invention by any well-known, in vitro or in vivo cleavage assay, such as those described in the International PCT Application WO 2004/067736; Epinat et al., Nucleic Acids Res., 2003, 31, 2952-2962; Chames et al., Nucleic Acids Res., 2005, 33, e178; Arnould et al., J. Mol. Biol., 2006, 355, 443-458, and Arnould et al., J. Mol. Biol., 2007, 371, 49-65. For example, the cleavage activity of the variant of the invention may be measured by a direct repeat recombination assay, in yeast or mammalian cells, using a reporter vector. The reporter vector comprises two truncated, non-functional copies of a reporter gene (direct repeats) and the genomic (non-palindromic) DNA target sequence within the intervening sequence, cloned in yeast or in a mammalian expression vector. Usually, the genomic DNA target sequence comprises one different half of each (palindromic or pseudo-palindromic) parent homodimeric I-CreI meganuclease target sequence. Expression of the heterodimeric variant results in a functional endonuclease which is able to cleave the genomic DNA target sequence. This cleavage induces homologous recombination between the direct repeats, resulting in a functional reporter gene, whose expression can be monitored by an appropriate assay. The cleavage activity of the variant against the genomic DNA target may be compared to wild type I-CreI or I-SceI activity against their natural target.

According to another advantageous embodiment of said method, steps (c), (d), (e), (0 and/or (j) are performed in vivo, under conditions where the double-strand break in the mutated DNA target sequence which is generated by said variant leads to the activation of a positive selection marker or a reporter gene, or the inactivation of a negative selection marker or a reporter gene, by recombination-mediated repair of said DNA double-strand break.

Furthermore, the homodimeric combined variants obtained in step (g) or (h) are advantageously submitted to a selection/screening step to identify those which are able to cleave a pseudo-palindromic sequence wherein at least the nucleotides at positions −11 to −3 (combined variant of step (g)) or +3 to +11 (combined variant of step (h)) are identical to the nucleotides which are present at positions −11 to −3 (combined variant of step (g)) or +3 to +11 (combined variant of step (h)) of said genomic target, and the nucleotides at positions +3 to +11 (combined variant of step (g)) or −11 to −3 (combined variant of step (h)) are identical to the reverse complementary sequence of the nucleotides which are present at positions −11 to −3 (combined variant of step (g)) or +3 to +11 (combined variant of step (h)) of said genomic target.

Preferably, the set of combined variants of step (g) or step (h) (or both sets) undergoes an additional selection/screening step to identify the variants which are able to cleave a pseudo-palindromic sequence wherein:

(1) the nucleotides at positions −11 to −3 (combined variant of step (g)) or +3 to +11 (combined variant of step (h)) are identical to the nucleotides which are present at positions −11 to −3 (combined variant of step (g)) or +3 to +11 (combined variant of step h)) of said genomic target, and

(2) the nucleotides at positions +3 to +11 (combined variant of step (g)) or −11 to −3 (combined variant of step (h)) are identical to the reverse complementary sequence of the nucleotides which are present at positions −11 to −3 (combined variant of step (g)) or +3 to +11 (combined variant of step (h)) of said genomic target.

This additional screening step increases the probability of isolating heterodimers which are able to cleave the genomic target of interest (step (k)).

Steps (a), (b), (g), (h) and (i) may further comprise the introduction of additional mutations at other positions contacting the DNA target sequence or interacting directly or indirectly with said DNA target, at positions which improve the binding and/or cleavage properties of the variants, or at positions which either prevent or impair the formation of functional homodimers or favor the formation of the heterodimer, as defined above.

The additional mutations may be introduced by site-directed mutagenesis and/or random mutagenesis on a variant or on a pool of variants, according to standard mutagenesis methods which are well-known in the art, for example by using PCR.

In particular, random mutations may be introduced into the whole variant or in a part of the variant to improve the binding and/or cleavage properties of the variants towards the DNA target from the gene of interest.

Site-directed mutagenesis at positions which improve the binding and/or cleavage properties of the variants, for example at positions 19, 54, 66, 80, 87, 105 and/or 132, may also be combined with random-mutagenesis. The mutagenesis may be performed by generating random/site-directed mutagenesis libraries on a pool of variants, according to standard mutagenesis methods which are well-known in the art. Site-directed mutagenesis may be advantageously performed by amplifying overlapping fragments comprising the mutated position(s), as defined above, according to well-known overlapping PCR techniques. In addition, multiple site-directed mutagenesis, may advantageously be performed on a variant or on a pool of variants.

Preferably, the mutagenesis is performed on one monomer of the heterodimer formed in step (i) or step (j), advantageously on a pool of monomers, preferably on both monomers of the heterodimer of step (i) or (j).

Possibly or not, at least two rounds of selection/screening are performed according to the process illustrated Arnould et al., J. Mol. Biol., 2007, 371, 49-65. In the first round, one of the monomers of the heterodimer is mutagenised, co-expressed with the other monomer to form heterodimers, and the improved monomers Y⁺ are selected against the target from the gene of interest. In the second round, the other monomer (monomer X) is mutagenised, co-expressed with the improved monomers Y⁺ to form heterodimers, and selected against the target from the gene of interest to obtain meganucleases (X⁺ Y⁺) with improved activity. The mutagenesis may be random-mutagenesis or site-directed mutagenesis on a monomer or on a pool of monomers, as indicated above. Both types of mutagenesis are advantageously combined. Additional rounds of selection/screening on one or both monomers may be performed to improve the cleavage activity of the variant.

Preferably the variant may be obtained by a method comprising the additional steps of:

(k) selecting heterodimers from step (j) and constructing a third series of variants having at least one substitution in at least one of the monomers in said selected heterodimers,

(l) combining said third series variants of step (k) and screening the resulting heterodimers for altered cleavage activity against said DNA target from DMD gene.

Preferably in step (k) at least one substitution is introduced by site directed mutagenesis in a DNA molecule encoding said third series of variants, and/or by random mutagenesis in a DNA molecule encoding said third series of variants.

Preferably steps (k) and (l) are repeated at least two times and wherein the heterodimers selected in step (k) of each further iteration are selected from heterodimers screened in step (l) of the previous iteration which showed altered cleavage activity against said DNA target from DMD gene.

Given the large size of the DMD gene and the large diversity of mutations resulting in Duchenne's Muscular Dystrophy, among which, a variety of deletions and duplications, the exon KI strategy is the most adapted to correct this gene in a large number of cases. However, even with this strategy, limitations linked to the maximal size of the sequences that can be inserted into existing vectors have to be envisioned.

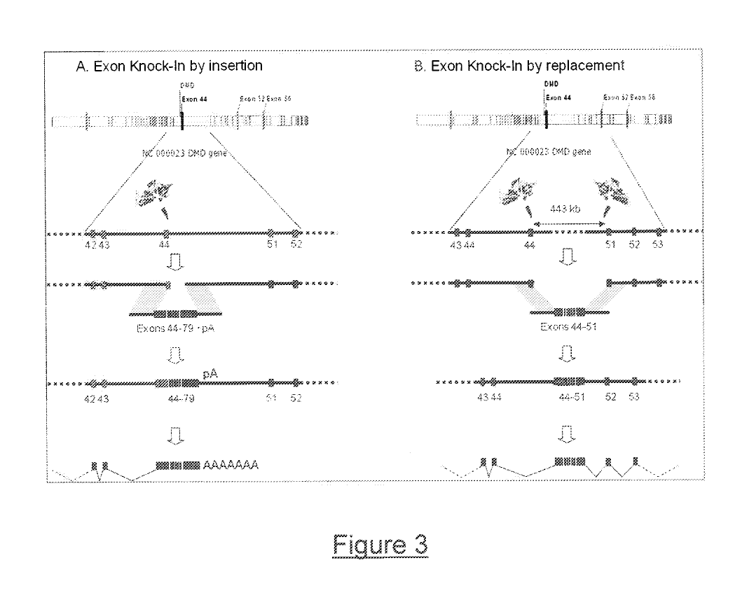

The inventors envision two different sub-types of exon KI strategies: in a first one, one would insert at a “starting point” a partial cDNA, providing all the exons downstream of this insertion point. This starting point has been placed in exon 44, or in the exons just upstream (FIG. 3-A). This strategy would address up to 60% of the existing mutations. It would require the insertion of a 4.8 kb sequence, corresponding to the downstream exons. The repair matrix would in addition have to include 1 kb of homology on each side (in the flanking introns), resulting in a fragment of about 7 kbs. This size remains compatible with the use of lentiviral vectors, and to a certain extent, with the use of AAV vectors for research purpose (although inserts up to 7 kb have been reported in such AAV vector, such long inserts should dramatically reduce the yield of large scale productions).

As a consequence, a cleavage 3′ of exon 44 can induce a gene targeting event with one breakpoint in the exon just 5′ of the break, i.e., in exon 44, and another one in the part of the intron just 3′ of the break. The resulting recombination event is described in FIG. 3-A. Importantly, recombination should occur between large homology regions, in intronic sequences (from intron 43 and 44). The presence of shorter stretches of homology between the exons of the cDNA to be knocked in and the endogenous exons should not interfere with the process, given the small size of the exons. In a similar approach, meganucleases targeting sequences in 3′ of former exons could be used to induce gene targeting events in exons 5′ of exon 44.

Thus, cleavage in the DMD21, DMD24, DMD31, DMD33, DMD35 and DMD37 sequences described in Table 1 could be used to induce gene targeting events with junctions in exons 38, 39, 42, 44, 51 and 53 respectively. The repair matrix would have to be in the range of 6.8 to 7.9 kb (i.e., about 5.9 kbs for exons 38-79, or 4.8 kbs for exons 44-79, with in addition 1 kb of homologous sequence on each side).

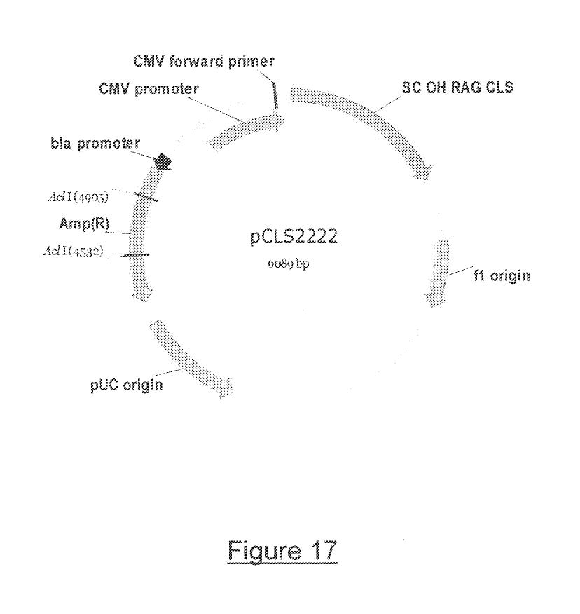

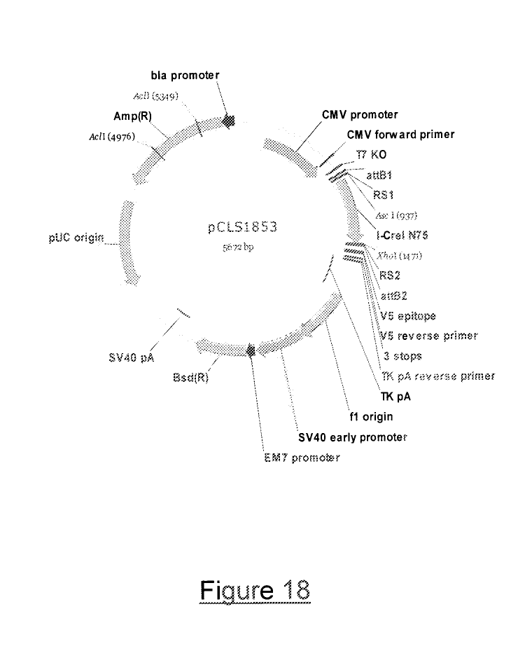

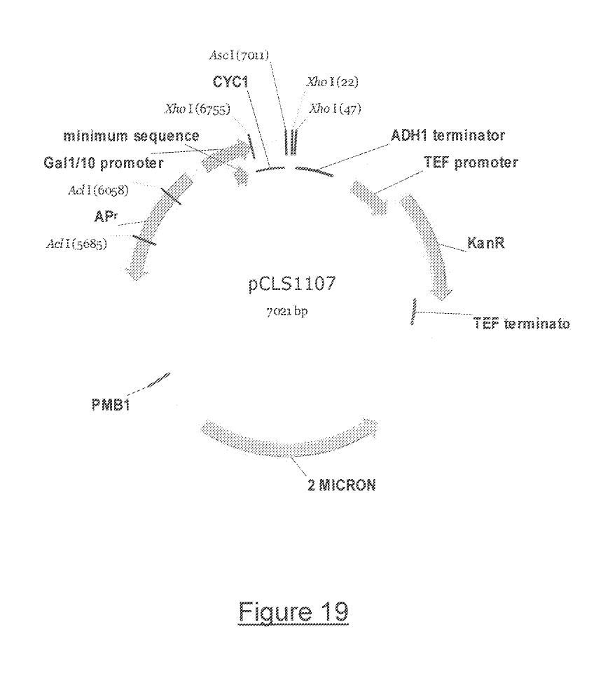

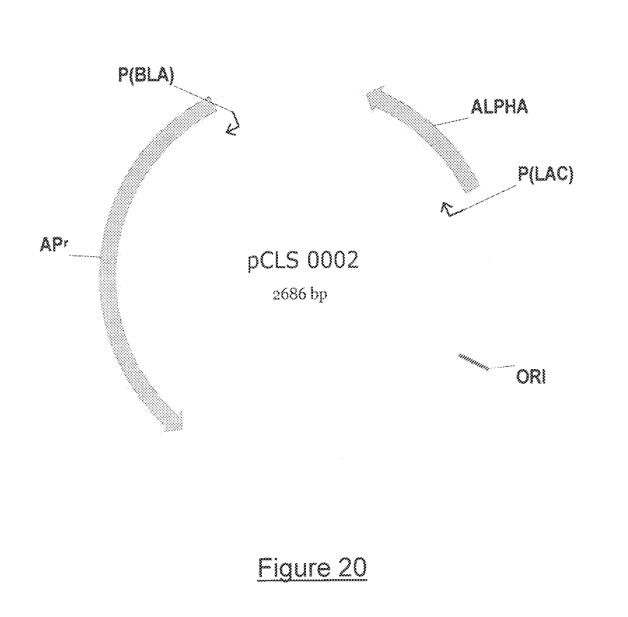

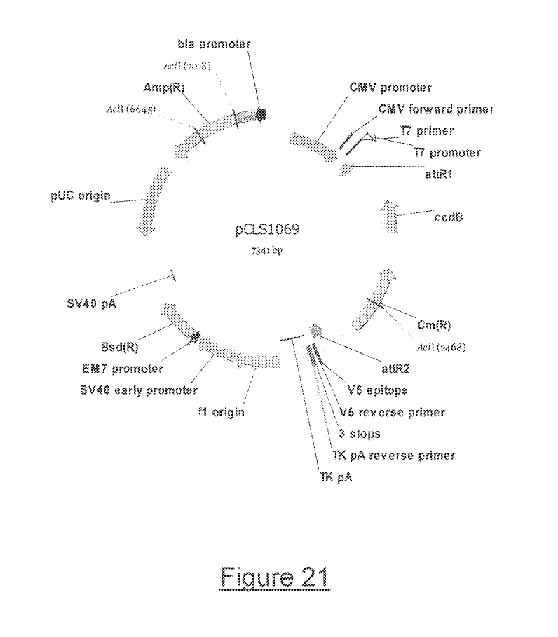

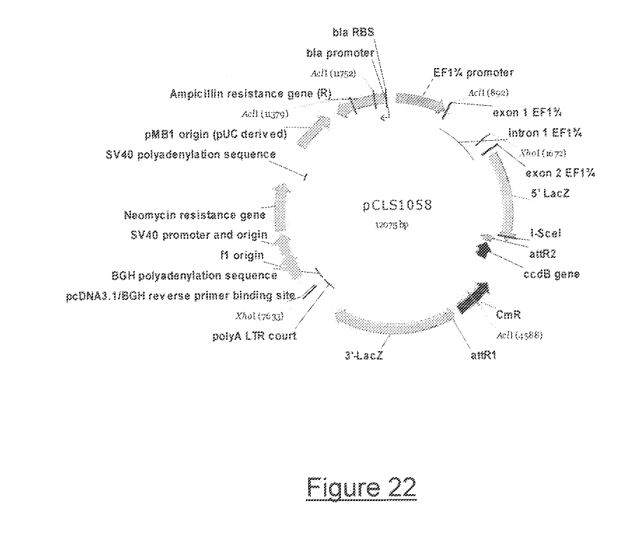

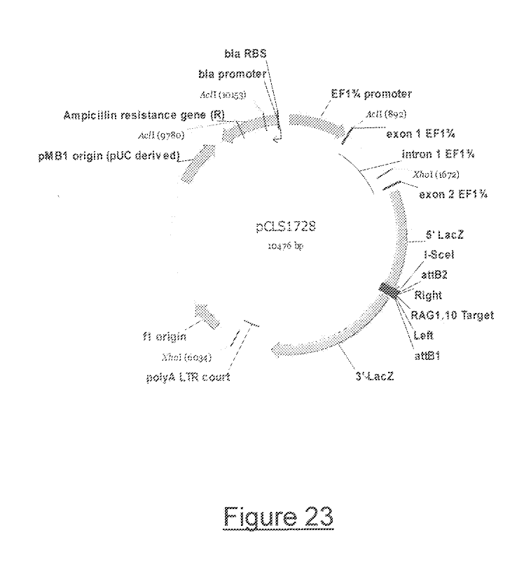

A second sub-type of exon knock-in strategy consists in the replacement of a very large region with a cDNA, requiring a second break in the chromosome, 5′ of a downstream exon that would represent the second breakpoint or junction of the recombination event (FIG. 3-B). This second breakpoint has been placed after exon 50. This strategy would address up to 30-40% of the existing mutations, and would require the insertion of a 1.2 kb sequence for exons 44 to 51 (3.2 kb repair matrix) and up to 2.5 kb for exons 38 to 53 (4.5 kb repair matrix). The replacement strategy is more “elegant” than the insertion, for it avoids duplications within the genome that could result in expression issues (repeated sequences may trigger gene inactivation). In addition, it would allow for the use of a smaller repair matrix. This size of the insert used here is also compatible with the use of lentiviral vectors, and with the use of meganuclease-induced recombination. The major unknown factor is actually the efficiency of recombination involving two chromosomal breakpoints placed several hundreds of Kb away. It has been demonstrated before that two I-SceI breaks located a few kbs away could induce efficient recombination in a process mimicking the one described in FIG. 17A (refs 30-31). Moreover, recombination involving rejoining of two I—SceI induced DSBs separated by 200 kb of sequences have been described (ref 32), and even breaks placed on different chromosomes have been shown to interact very efficiently (refs 33-34). For DMD, the target cells could be mesoangioblasts, which can be grafted by systemic injection. Another option is the targeting of myoblasts, although these cells need to be grafted locally.

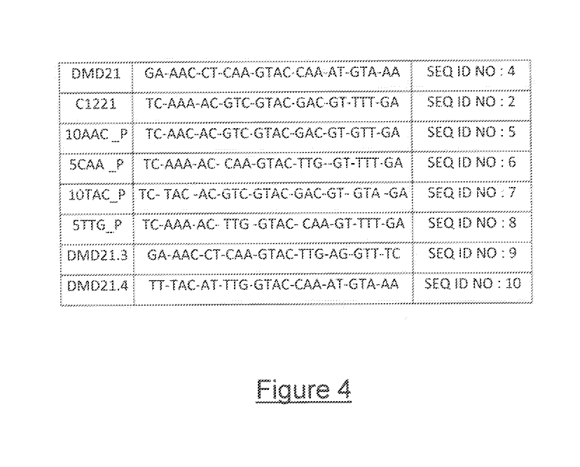

I-CreI variants to these targets were created using a combinatorial approach, to entirely redesign the DNA binding domain of the I-CreI protein and thereby engineer novel meganucleases with fully engineered specificity for the desired DMD gene target. Some of the DNA targets identified by the inventors to validate their invention are given in the table I below. Derivatives of these DNA targets are given in FIGS. 4, 6, 8, 10, 12 and 14.

TABLE I
sequences and location of the targeted sites in the DMD gene

| mega | position | targeted sequence | Target for KI |
|---|---|---|---|
| DMD21 | 993350-993373 | GA-AAC-CT-CAA-GTAC-CAA-AT-GTA-AA | 3′ of exon 38 |
| | Intron 38 | | |
| DMD24 | 995930-995953 | TT-TAC-CT-ATT-TTAA-GTC-AG-ATA-CA | 3′ of exon 39 |
| | Intron 39 | | |
| DMD33 | 1031834-1031857 | AA-ATC-CT-GCC-TTAA-AGT-AT-CTC-AT | 3′ of exon 42 |
| | Intron 42 | | |
| DMD31 | 1125314-1125337 | AA-TGT-CT-GAT-GTTC-AAT-GT-GTT-GA | 3′ of exon 44 |
| | Intron 44 | | |
| DMD35 | 1561221-1561244 | TC-TTT-AT-GTT-TTAA-AGT-AT-ATT-CC | 5′ of exon 51 |
| | Intron 50 | | |
| DMD37 | 1659873-1659896 | GA-ATC-CT-GTT-GTTC-ATC-AT-CCT-AG | 5′ of exon 53 |
| | Intron 52 | | |

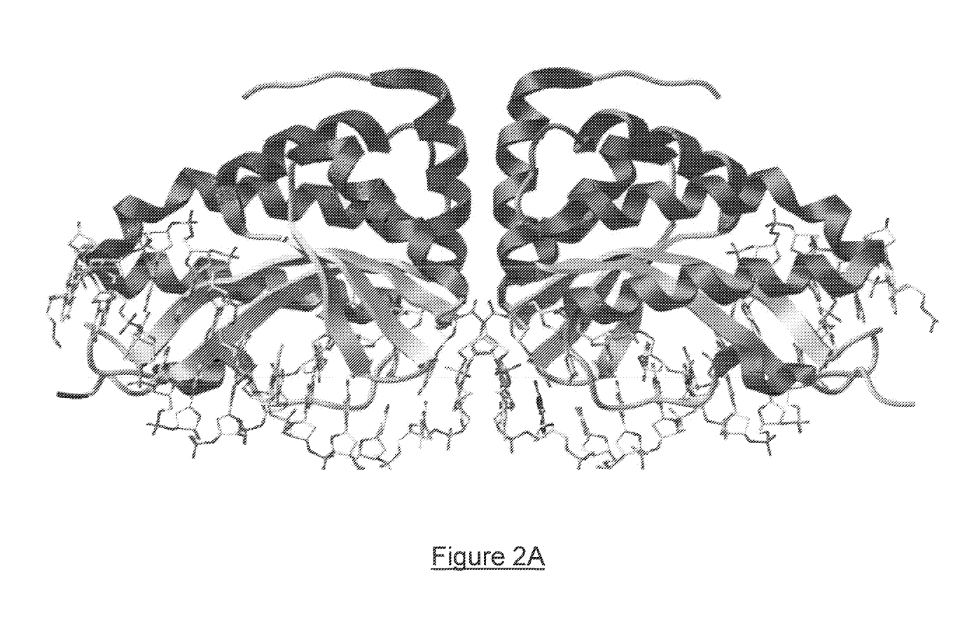

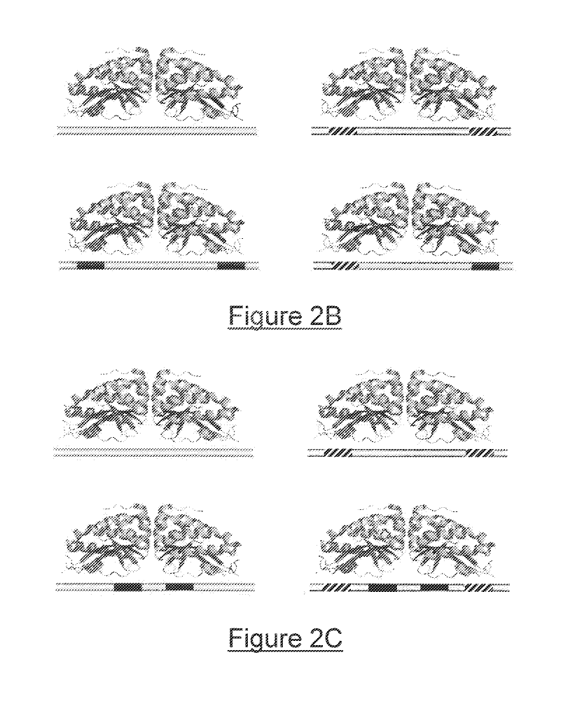

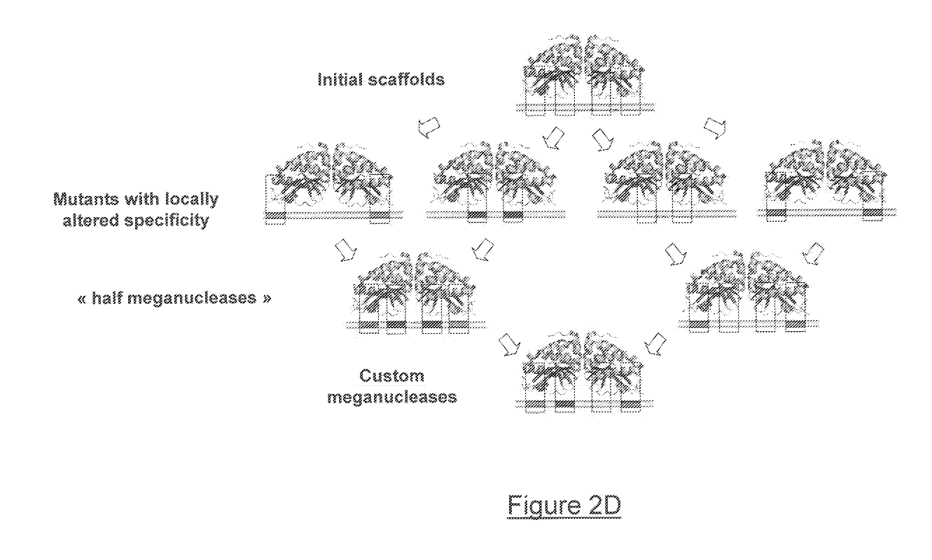

The combinatorial approach, as illustrated in FIG. 2D was used to entirely redesign the DNA binding domain of the I-CreI protein and thereby engineer novel meganucleases with fully engineered specificity.

In particular the heterodimer of step (i) may comprise monomers obtained in steps (g) and (h), with the same DNA target recognition and cleavage activity properties.

Alternatively the heterodimer of step (i) may comprise monomers obtained in steps (g) and (h), with different DNA target recognition and cleavage activity properties.

In particular the first series of I-CreI variants of step (a) are derived from a first parent meganuclease.

In particular the second series of variants of step (b) are derived from a second parent meganuclease.

In particular the first and second parent meganucleases are identical.

Alternatively the first and second parent meganucleases are different.

In particular the variant may be obtained by a method comprising the additional steps of:

(k) selecting heterodimers from step (j) and constructing a third series of variants having at least one substitution in at least one of the monomers of said selected heterodimers,

(l) combining said third series variants of step (k) and screening the resulting heterodimers for enhanced cleavage activity against said DNA target from DMD gene.

In a preferred embodiment of said variant, said substitution(s) in the subdomain situated from positions 44 to 77 of I-CreI are at positions 44, 68, 70, 75 and/or 77.

In another preferred embodiment of said variant, said substitution(s) in the subdomain situated from positions 28 to 40 of I-CreI are at positions 28, 30, 32, 33, 38 and/or 40.

In another preferred embodiment of said variant, it comprises one or more mutations in I-CreI monomer(s) at positions of other amino acid residues that contact the DNA target sequence or interact with the DNA backbone or with the nucleotide bases, directly or via a water molecule; these residues are well-known in the art (Jurica et al., Molecular Cell., 1998, 2, 469-476; Chevalier et al., J. Mol. Biol., 2003, 329, 253-269). In particular, additional substitutions may be introduced at positions contacting the phosphate backbone, for example in the final C-terminal loop (positions 137 to 143; Prieto et al., Nucleic Acids Res., Epub 22 Apr. 2007).

Preferably said residues are involved in binding and cleavage of said DNA cleavage site.

More preferably, said residues are at positions 138, 139, 142 or 143 of I-CreI. Two residues may be mutated in one variant provided that each mutation is in a different pair of residues chosen from the pair of residues at positions 138 and 139 and the pair of residues at positions 142 and 143. The mutations which are introduced modify the interaction(s) of said amino acid(s) of the final C-terminal loop with the phosphate backbone of the I-CreI site. Preferably, the residue at position 138 or 139 is substituted by a hydrophobic amino acid to avoid the formation of hydrogen bonds with the phosphate backbone of the DNA cleavage site. For example, the residue at position 138 is substituted by an alanine or the residue at position 139 is substituted by a methionine. The residue at position 142 or 143 is advantageously substituted by a small amino acid, for example a glycine, to decrease the size of the side chains of these amino acid residues.

More preferably, said substitution in the final C-terminal loop modify the specificity of the variant towards the nucleotide at positions ±1 to 2, ±6 to 7 and/or ±11 to 12 of the I-CreI site.

In another preferred embodiment of said variant, it comprises one or more additional mutations that improve the binding and/or the cleavage properties of the variant towards the DNA target sequence from the DMD gene. The additional residues which are mutated may be on the entire I-CreI sequence, and in particular in the C-terminal half of I-CreI (positions 80 to 163). Both I-CreI monomers are advantageously mutated; the mutation(s) in each monomer may be identical or different. For example, the variant comprises one or more additional substitutions at positions: 2, 19, 43, 80 and 81. Said substitutions are advantageously selected from the group consisting of: N2S, G19S, F43L, E80K and I81T. More preferably, the variant comprises at least one substitution selected from the group consisting of: N2S, G19S, F43L, E80K and I81T. The variant may also comprise additional residues at the C-terminus. For example a glycine (G) and/or a proline (P) residue may be inserted at positions 164 and 165 of I-CreI, respectively.

According to a preferred embodiment, said additional mutation in said variant further impairs the formation of a functional homodimer. More preferably, said mutation is the G19S mutation. The G19S mutation is advantageously introduced in one of the two monomers of a heterodimeric I-CreI variant, so as to obtain a meganuclease having enhanced cleavage activity and enhanced cleavage specificity. In addition, to enhance the cleavage specificity further, the other monomer may carry a distinct mutation that impairs the formation of a functional homodimer or favors the formation of the heterodimer.

In another preferred embodiment of said variant, said substitutions are replacement of the initial amino acids with amino acids selected from the group consisting of: A, D, E, G, H, K, N, P, Q, R, S, T, Y, C, V, L, M, F, I and W.

In particular the variant is selected from the group consisting of SEQ ID NO: 40 to 65.

The variant of the invention may be derived from the wild-type I-CreI (SEQ ID NO: 1) or an I-CreI scaffold protein having at least 85% identity, preferably at least 90% identity, more preferably at least 95% identity with SEQ ID NO: 1, such as the scaffold called I-CreI N75 (167 amino acids; SEQ ID NO: 3) having the insertion of an alanine at position 2, and the insertion of AAD at the C-terminus (positions 164 to 166) of the I-CreI sequence. In the present patent application all the I-CreI variants described comprise an additional Alanine after the first Methionine of the wild type I-CreI sequence (SEQ ID NO: 1). These variants also comprise two additional Alanine residues and an Aspartic Acid residue after the final Proline of the wild type I-CreI sequence. These additional residues do not affect the properties of the enzyme and to avoid confusion these additional residues do not affect the numeration of the residues in I-CreI or a variant referred in the present patent application, as these references exclusively refer to residues of the wild type I-CreI enzyme (SEQ ID NO: 1) as present in the variant, so for instance residue 2 of I-CreI is in fact residue 3 of a variant which comprises an additional Alanine after the first Methionine.

In addition, the variants of the invention may include one or more residues inserted at the NH₂ terminus and/or COOH terminus of the sequence. For example, a tag (epitope or polyhistidine sequence) is introduced at the NH₂ terminus and/or COOH terminus; said tag is useful for the detection and/or the purification of said variant. The variant may also comprise a nuclear localization signal (NLS); said NLS is useful for the importation of said variant into the cell nucleus. The NLS may be inserted just after the first methionine of the variant or just after an N-terminal tag.

The variant according to the present invention may be a homodimer which is able to cleave a palindromic or pseudo-palindromic DNA target sequence.

Alternatively, said variant is a heterodimer, resulting from the association of a first and a second monomer having different substitutions at positions 28 to 40 and 44 to 77 of 1-CreI, said heterodimer being able to cleave a non-palindromic DNA target sequence from the DMD gene.

In particular said heterodimer variant is composed by one of the possible associations between variants constituting N-terminal and C-terminal monomers of single chain molecules from the group consisting of SEQ ID NO: 62 to SEQ ID NO: 105, SEQ ID NO: 116 to SEQ ID NO: 119, SEQ ID NO: 121 and SEQ ID NO: 122 to SEQ ID NO: 130.

The DNA target sequences are situated in the DMD Open Reading Frame (ORF) and these sequences cover all the DMD ORF. In particular, said DNA target sequences for the variant of the present invention and derivatives are selected from the group consisting of the SEQ ID NO: 4 to SEQ ID NO: 60, as shown in FIGS. 4, 6, 8, 10, 12 and 14 and Table I.

The sequence of each I-CreI variant is defined by the mutated residues at the indicated positions. The positions are indicated by reference to I-CreI sequence (SEQ ID NO: 1); I-CreI has N, S, Y, Q, S, Q, R, R, D, I and E at positions 30, 32, 33, 38, 40, 44, 68, 70, 75, 77 and 80 respectively.

Each monomer (first monomer and second monomer) of the heterodimeric variant according to the present invention may also be named with a letter code, after the eleven residues at positions 28, 30, 32, 33, 38, 40, 44, 68 and 70, 75 and 77 and the additional residues which are mutated, as indicated above. For example, the mutations 7E30R40E44T46G68T70S73M75A77R80K96E132V154N in the N-terminal monomer constituting a single chain molecule targeting the DMD21 target of the present invention (SEQ ID NO: 64).

In the present invention, for a given DNA target, “0.2” derivative target sequence differs from the initial genomic target at positions −2, −1, +1, +2, where I-CreI cleavage site (GTAC) substitutes the corresponding sequence at these positions of said initial genomic target. “0.3” derivative target sequence is the palindromic sequence derived from the left part of said “0.2” derivative target sequence. “0.4” derivative target sequence is the palindromic sequence derived from the right part of said “0.2” derivative target sequence. “0.5” derivative target sequence is the palindromic sequence derived from the left part of the initial genomic target. “0.6” derivative is the palindromic sequence derived from the left part of the initial genomic target. As an illustrative example, for DMD 24 (FIG. 4), “DMD24.2” derivative target sequence differs from the initial genomic target (DMD24) at positions −2, −1, +1, +2, where I-CreI cleavage site (GTAC) substitutes the corresponding sequence at these positions of said initial genomic target (DMD24). “DMD24.3” derivative target sequence is the palindromic sequence derived from the left part of said “DMD24.2” derivative target sequence. “DMD24.4” derivative target sequence is the palindromic sequence derived from the right part of said “DMD24.2” derivative target sequence. “DMD24.5” derivative target sequence is the palindromic sequence derived from the left part of the initial genomic target (DMD24). “DMD24.6” derivative is the palindromic sequence derived from the right part of the initial genomic target (DMD24).

In the present invention, a “N-terminal monomer” constituting one of the monomers of a single chain molecule, refers to a variant able to cleave “0.3” or “0.5” palindromic sequence. In the present invention, a “C-terminal monomer” constituting one of the monomers of a single chain molecule, refers to a variant able to cleave “0.4” or “0.6” palindromic sequence.

The heterodimeric variant as defined above may have only the amino acid substitutions as indicated above. In this case, the positions which are not indicated are not mutated and thus correspond to the wild-type I-CreI (SEQ ID NO: 1).

The invention encompasses I-CreI variants having at least 85% identity, preferably at least 90% identity, more preferably at least 95% (96%, 97%, 98%, 99%) identity with the sequences as defined above, said variant being able to cleave a DNA target from the DMD gene.

The heterodimeric variant is advantageously an obligate heterodimer variant having at least one pair of mutations corresponding to residues of the first and the second monomers which make an intermolecular interaction between the two I-CreI monomers, wherein the first mutation of said pair(s) is in the first monomer and the second mutation of said pair(s) is in the second monomer and said pair(s) of mutations prevent the formation of functional homodimers from each monomer and allow the formation of a functional heterodimer, able to cleave the genomic DNA target from the DMD gene.

To form an obligate heterodimer, the monomers have advantageously at least one of the following pairs of mutations, respectively for the first monomer and the second monomer:

a) the substitution of the glutamic acid at position 8 with a basic amino acid, preferably an arginine (first monomer) and the substitution of the lysine at position 7 with an acidic amino acid, preferably a glutamic acid (second monomer); the first monomer may further comprise the substitution of at least one of the lysine residues at positions 7 and 96, by an arginine,

b) the substitution of the glutamic acid at position 61 with a basic amino acid, preferably an arginine (first monomer) and the substitution of the lysine at position 96 with an acidic amino acid, preferably a glutamic acid (second monomer); the first monomer may further comprise the substitution of at least one of the lysine residues at positions 7 and 96, by an arginine,

c) the substitution of the leucine at position 97 with an aromatic amino acid, preferably a phenylalanine (first monomer) and the substitution of the phenylalanine at position 54 with a small amino acid, preferably a glycine (second monomer); the first monomer may further comprise the substitution of the phenylalanine at position 54 by a tryptophane and the second monomer may further comprise the substitution of the leucine at position 58 or lysine at position 57, by a methionine, and

d) the substitution of the aspartic acid at position 137 with a basic amino acid, preferably an arginine (first monomer) and the substitution of the arginine at position 51 with an acidic amino acid, preferably a glutamic acid (second monomer).

For example, the first monomer may have the mutation D137R and the second monomer, the mutation R51D. The obligate heterodimer meganuclease comprises advantageously, at least two pairs of mutations as defined in a), b), c) or d), above; one of the pairs of mutation is advantageously as defined in c) or d). Preferably, one monomer comprises the substitution of the lysine residues at positions 7 and 96 by an acidic amino acid (aspartic acid (D) or glutamic acid (E)), preferably a glutamic acid (K7E and K96E) and the other monomer comprises the substitution of the glutamic acid residues at positions 8 and 61 by a basic amino acid (arginine (R) or lysine (K); for example, E8K and E61R). More preferably, the obligate heterodimer meganuclease, comprises three pairs of mutations as defined in a), b) and c), above.

The obligate heterodimer meganuclease consists advantageously of a first monomer (A) having at least the mutations (i) E8R, E8K or E8H, E61R, E61K or E61H and L97F, L97W or L97Y; (ii) K7R, E8R, E61R, K96R and L97F, or (iii) K7R, E8R, F54W, E61R, K96R and L97F and a second monomer (B) having at least the mutations (iv) K7E or K7D, F54G or F54A and K96D or K96E; (v) K7E, F54G, L58M and K96E, or (vi) K7E, F54G, K57M and K96E. For example, the first monomer may have the mutations K7R, E8R or E8K, E61R, K96R and L97F or K7R, E8R or E8K, F54W, E61R, K96R and L97F and the second monomer, the mutations K7E, F54G, L58M and K96E or K7E, F54G, K57M and K96E. The obligate heterodimer may comprise at least one NLS and/or one tag as defined above; said NLS and/or tag may be in the first and/or the second monomer.

The subject-matter of the present invention is also a single-chain chimeric meganuclease (fusion protein) derived from an I-CreI variant as defined above. The single-chain meganuclease may comprise two I-CreI monomers, two I-CreI core domains (positions 6 to 94 of I-CreI) or a combination of both. Preferably, the two monomers/core domains or the combination of both, are connected by a peptidic linker. Said peptidic linker can be RM2 linker (SEQ ID NO: 61) or BQY linker (SEQ ID NO: 120) or another suitable linker.

More preferably the single-chain chimeric meganuclease is composed by one of the possible associations between variants from the group consisting of N-terminal monomers and C-terminal monomers, given in Tables II to VII, respectively for a given DNA target, DMD21, DMD24, DMD31, DMD33, DMD35 and DMD37, said monomer variants being connected by a linker. More preferably the single-chain chimeric meganuclease according to the present invention is one from the group consisting of SEQ ID NO: 62 to SEQ ID NO: 105, SEQ ID NO: 116 to SEQ ID NO: 119, SEQ ID NO: 121 and SEQ ID NO: 122 to SEQ ID NO: 130. Regarding DMD21 target, the single-chain chimeric meganuclease according to the present invention is one from the group consisting of SEQ ID NO: 62 to SEQ ID NO: 68 and SEQ ID NO: 116 to SEQ ID NO: 119. Regarding DMD24 target, the single-chain chimeric meganuclease according to the present invention is one from the group consisting of SEQ ID NO: 69 to SEQ ID NO: 77. Regarding DMD31 target, the single-chain chimeric meganuclease according to the present invention is one from the group consisting of SEQ ID NO: 78 to SEQ ID NO: 84. Regarding DMD33 target, the single-chain chimeric meganuclease according to the present invention is one from the group consisting of SEQ ID NO: 85 to SEQ ID NO: 95. Regarding DMD35 target, the single-chain chimeric meganuclease according to the present invention is one from the group consisting of SEQ ID NO: 96 to SEQ ID NO: 99 and SEQ ID NO: 121. Regarding DMD37 target, the single-chain chimeric meganuclease according to the present invention is one from the group consisting of SEQ ID NO: 100 to SEQ ID NO: 105 and SEQ ID NO: 122 to SEQ ID NO: 130.

It is understood that the scope of the present invention also encompasses the I-CreI variants per se, including heterodimers, obligate heterodimers, single chain meganucleases as non limiting examples, able to cleave one of the sequence targets in DMD gene.

The subject-matter of the present invention is also a polynucleotide fragment encoding a variant or a single-chain chimeric meganuclease as defined above; said polynucleotide may encode one monomer of a homodimeric or heterodimeric variant, or two domains/monomers of a single-chain chimeric meganuclease. It is understood that the subject-matter of the present invention is also a polynucleotide fragment encoding one of the variant species as defined above, obtained by any method well-known in the art.

The subject-matter of the present invention is also a recombinant vector for the expression of a variant or a single-chain meganuclease according to the invention. The recombinant vector comprises at least one polynucleotide fragment encoding a variant or a single-chain meganuclease, as defined above. In a preferred embodiment, said vector comprises two different polynucleotide fragments, each encoding one of the monomers of a heterodimeric variant.

A vector which can be used in the present invention includes, but is not limited to, a viral vector, a plasmid, a RNA vector or a linear or circular DNA or RNA molecule which may consists of a chromosomal, non chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those skilled in the art and commercially available.

Viral vectors include retrovirus, adenovirus, parvovirus (e.g. adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus (particularly self inactivacting lentiviral vectors), spumavirus (Coffin, J. M., Retroviridae: The viruses and their replication, In Fundamental Virology, Third Edition, B. N. Fields, et al., Eds., Lippincott-Raven Publishers, Philadelphia, 1996).

Vectors can comprise selectable markers, for example: neomycin phosphotransferase, histidinol dehydrogenase, dihydrofolate reductase, hygromycin phosphotransferase, herpes simplex virus thymidine kinase, adenosine deaminase, Glutamine Synthetase, and hypoxanthine-guanine phosphoribosyl transferase for eukaryotic cell culture; TRP1, URA3 and LEU2 for S. cerevisiae; tetracycline, rifampicin or ampicillin resistance in E. coli.

Preferably said vectors are expression vectors, wherein the sequence(s) encoding the variant/single-chain meganuclease of the invention is placed under control of appropriate transcriptional and translational control elements to permit production or synthesis of said variant. Therefore, said polynucleotide is comprised in an expression cassette. More particularly, the vector comprises a replication origin, a promoter operatively linked to said polynucleotide, a ribosome-binding site, an RNA-splicing site (when genomic DNA is used), a polyadenylation site and a transcription termination site. It also can comprise an enhancer. Selection of the promoter will depend upon the cell in which the polypeptide is expressed. Preferably, when said variant is a heterodimer, the two polynucleotides encoding each of the monomers are included in one vector which is able to drive the expression of both polynucleotides, simultaneously. Suitable promoters include tissue specific and/or inducible promoters. Examples of inducible promoters are: eukaryotic metallothionine promoter which is induced by increased levels of heavy metals, prokaryotic lacZ promoter which is induced in response to isopropyl-β-D-thiogalacto-pyranoside (IPTG) and eukaryotic heat shock promoter which is induced by increased temperature. Examples of tissue specific promoters are skeletal muscle creatine kinase, prostate-specific antigen (PSA), α-antitrypsin protease, human surfactant (SP) A and B proteins, β-casein and acidic whey protein genes.

According to another advantageous embodiment of said vector, it includes a targeting construct comprising sequences sharing homologies with the region surrounding the genomic DNA cleavage site as defined above.

For instance, said sequence sharing homologies with the regions surrounding the genomic DNA cleavage site of the variant is a fragment of the DMD gene. Alternatively, the vector coding for an I-CreI variant/single-chain meganuclease and the vector comprising the targeting construct are different vectors.

More preferably, the targeting DNA construct comprises:

a) sequences sharing homologies with the region surrounding the genomic DNA cleavage site as defined above, and

b) a sequence to be introduced flanked by sequences as in a) or included in sequences as in a).

Preferably, homologous sequences of at least 50 bp, preferably more than 100 bp and more preferably more than 200 bp are used. Therefore, the targeting DNA construct is preferably from 200 bp to 6000 bp, more preferably from 1000 bp to 2000 bp. Indeed, shared DNA homologies are located in regions flanking upstream and downstream the site of the break and the DNA sequence to be introduced should be located between the two arms. The sequence to be introduced may be any sequence used to alter the chromosomal DNA in some specific way including a sequence used to repair a mutation in the DMD gene, restore a functional DMD gene in place of a mutated one, modify a specific sequence in the DMD gene, to attenuate or activate the DMD gene, to inactivate or delete the DMD gene or part thereof, to introduce a mutation into a site of interest or to introduce an exogenous gene or part thereof. Such chromosomal DNA alterations are used for genome engineering (animal models/recombinant cell lines) or genome therapy (gene correction or recovery of a functional gene). The targeting construct comprises advantageously a positive selection marker between the two homology arms and eventually a negative selection marker upstream of the first homology arm or downstream of the second homology arm. The marker(s) allow(s) the selection of cells having inserted the sequence of interest by homologous recombination at the target site.

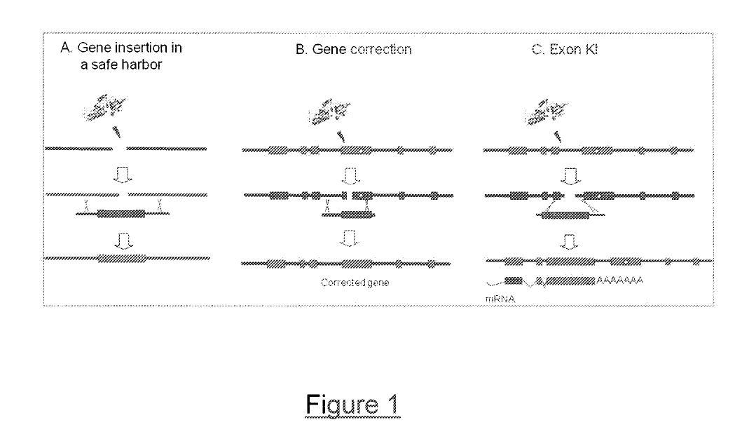

The sequence to be introduced is a sequence which repairs a mutation in the DMD gene (gene correction or recovery of a functional gene), for the purpose of genome therapy (FIGS. 1B and 1C). For correcting the DMD gene, cleavage of the gene occurs in the vicinity of the mutation, preferably, within 500 bp of the mutation (FIG. 1B). The targeting construct comprises a DMD gene fragment which has at least 200 bp of homologous sequence flanking the target site (minimal repair matrix) for repairing the cleavage, and includes a sequence encoding a portion of wild-type DMD gene corresponding to the region of the mutation for repairing the mutation (FIG. 1B). Consequently, the targeting construct for gene correction comprises or consists of the minimal repair matrix; it is preferably from 200 bp to 6000 bp, more preferably from 1000 bp to 2000 bp. Preferably, when the cleavage site of the variant overlaps with the mutation the repair matrix includes a modified cleavage site that is not cleaved by the variant which is used to induce said cleavage in the DMD gene and a sequence encoding wild-type DMD gene that does not change the open reading frame of the DMD gene.

Alternatively, for the generation of knock-in cells/animals, the targeting DNA construct may comprise flanking regions corresponding to DMD gene fragments which has at least 200 bp of homologous sequence flanking the target site of the I-CreI variant for repairing the cleavage, an exogenous gene of interest within an expression cassette and eventually a selection marker such as the neomycin resistance gene.

For the insertion of a sequence, DNA homologies are generally located in regions directly upstream and downstream to the site of the break (sequences immediately adjacent to the break; minimal repair matrix). However, when the insertion is associated with a deletion of ORF sequences flanking the cleavage site, shared DNA homologies are located in regions upstream and downstream the region of the deletion.

Alternatively, for restoring a functional gene (FIGS. 1B et 1C), cleavage of the gene occurs in the vicinity or upstream of a mutation. Preferably said mutation is the first known mutation in the sequence of the gene, so that all the downstream mutations of the gene can be corrected simultaneously. The targeting construct comprises the exons downstream of the cleavage site fused in frame (as in the cDNA) and with a polyadenylation site to stop transcription in 3′. The sequence to be introduced (exon knock-in construct) is flanked by introns or exons sequences surrounding the cleavage site, so as to allow the transcription of the engineered gene (exon knock-in gene) into a mRNA able to code for a functional protein (FIG. 1C). For example, the exon knock-in construct is flanked by sequences upstream and downstream of the cleavage site, from a minimal repair matrix as defined above.

The subject matter of the present invention is also a targeting DNA construct as defined above.

The subject-matter of the present invention is also a composition characterized in that it comprises at least one meganuclease as defined above (variant or single-chain chimeric meganuclease) and/or at least one expression vector encoding said meganuclease, as defined above.

In a preferred embodiment of said composition, it comprises a targeting DNA construct, as defined above.

Preferably, said targeting DNA construct is either included in a recombinant vector or it is included in an expression vector comprising the polynucleotide(s) encoding the meganuclease according to the invention.

The subject-matter of the present invention is further the use of a meganuclease as defined above, one or two polynucleotide(s), preferably included in expression vector(s), for repairing mutations of the dystrophin gene.

The subject-matter of the present invention is also further a method of treatment of a genetic disease caused by a mutation in DMD gene comprising administering to a subject in need thereof an effective amount of at least one variant encompassed in the present invention.

According to an advantageous embodiment of said use, it is for inducing a double-strand break in a site of interest of the DMD gene comprising a genomic DNA target sequence, thereby inducing a DNA recombination event, a DNA loss or cell death.

According to the invention, said double-strand break is for: repairing a specific sequence in the DMD gene, modifying a specific sequence in the DMD gene, restoring a functional DMD gene in place of a mutated one, attenuating or activating the DMD gene, introducing a mutation into a site of interest of the DMD gene, introducing an exogenous gene or a part thereof, inactivating or deleting the DMD gene or a part thereof, translocating a chromosomal arm, or leaving the DNA unrepaired and degraded.

Given the very large size of the DMD locus, it is unlikely that targeted insertion into this locus could result into cis-activation of other genes. However, it could disrupt the DMD gene itself. Therefore, one can consider the DMD locus as a safe harbor
(iii) In cells that do not normally express DMD, provided the insert can be expressed from this locus.
(iv) In cells that do normally express DMD, provided the insertion does not affect the expression of DMD, or provided there remain a functional allele in the cell. For example insertion in introns can be made with no or minor modification of the expression pattern.

Therefore, in a second main aspect of the present invention, the inventors have found that endonucleases variants targeting DMD gene can be used for inserting therapeutic transgenes other than DMD at the dystrophin gene locus, using this locus as a safe harbor locus. In other terms, the invention relates to a mutant endonuclease capable of cleaving a target sequence in DMD gene locus, for use in safely inserting a transgene, wherein said disruption or deletion of said locus does not modify expression of genes located outside of said locus, and/or the cellular proliferation and/or the growth rate of the cell, tissue or individual.

The subject-matter of the present invention is also further a method of treatment of a genetic disease caused by a mutation in a gene other than DMD gene comprising administering to a subject in need thereof an effective amount of at least one variant encompassed in the present invention.

Those skilled in the art can easily verify whether disruption or deletion of a locus modifies expression of genes located outside of said locus using proteomic tools. Many protein expression profiling arrays suitable for such an analysis are commercially available. In particular, disruption or deletion of the DMD gene locus does not modify expression of neighboring genes, i.e., of genes located at the vicinity of the DMD gene locus. By “neighboring genes” is meant the 1, 2, 5, 10, 20 or 30 genes that are located at each end of the DMD gene locus.

In a derived third main aspect of the present invention, the inventors have found that the dystrophin locus could be used as a landing pad to insert and express genes of interest (GOIs) other than therapeutics. In this aspect, inventors have found that genetic constructs containing a GOI could be integrated into the genome at the DMD gene locus via meganuclease-induced recombination by specific meganuclease variants targeting DMD gene locus according to the first aspect of the invention.

The subject-matter of the present invention is also further a method for inserting a transgene into the genomic DMD locus of a cell, tissue or non-human animal wherein at least one variant of claim 1 is introduced in said cell, tissue or non-human animal.

In a preferred embodiment, the DMD locus further allows stable expression of the transgene. In another preferred embodiment, the target sequence inside the DMD locus is only present once within the genome of said cell, tissue or individual.

In another preferred embodiment meganuclease variants according to the present invention can be part of a kit to introduce a sequence encoding a GOI into at least one cell. In a more preferred embodiment, the at least one cell is selected form the group comprising: CHO-K1 cells; HEK293 cells; Caco2 cells; U2-OS cells; NIH 3T3 cells; NSO cells; SP2 cells; CHO-S cells; DG44 cells; K-562 cells, U-937 cells; MRC5 cells; IMR90 cells; Jurkat cells; HepG2 cells; HeLa cells; HT-1080 cells; HCT-116 cells; Hu-h7 cells; Huvec cells; Molt 4 cells.

The subject-matter of the present invention is also a method for making a DMD gene knock-out or knock-in recombinant cell, comprising at least the step of:

(a) introducing into a cell, a meganuclease as defined above (I-CreI variant or single-chain derivative), so as to induce a double stranded cleavage at a site of interest of the DMD gene comprising a DNA recognition and cleavage site for said meganuclease, simultaneously or consecutively,

(b) introducing into the cell of step (a), a targeting DNA, wherein said targeting DNA comprises (1) DNA sharing homologies to the region surrounding the cleavage site and (2) DNA which repairs the site of interest upon recombination between the targeting DNA and the chromosomal DNA, so as to generate a recombinant cell having repaired the site of interest by homologous recombination,

(c) isolating the recombinant cell of step (b), by any appropriate means.

The subject-matter of the present invention is also a method for making a DMD gene knock-out or knock-in animal, comprising at least the step of:

(a) introducing into a pluripotent precursor cell or an embryo of an animal, a meganuclease as defined above, so as to induce a double stranded cleavage at a site of interest of the DMD gene comprising a DNA recognition and cleavage site for said meganuclease, simultaneously or consecutively,

(b) introducing into the animal precursor cell or embryo of step (a) a targeting DNA, wherein said targeting DNA comprises (1) DNA sharing homologies to the region surrounding the cleavage site and (2) DNA which repairs the site of interest upon recombination between the targeting DNA and the chromosomal DNA, so as to generate a genetically modified animal precursor cell or embryo having repaired the site of interest by homologous recombination,

(c) developing the genetically modified animal precursor cell or embryo of step (b) into a chimeric animal, and

(d) deriving a transgenic animal from the chimeric animal of step (c).

Preferably, step (c) comprises the introduction of the genetically modified precursor cell generated in step (b) into blastocysts so as to generate chimeric animals.

The targeting DNA is introduced into the cell under conditions appropriate for introduction of the targeting DNA into the site of interest.

For making knock-out cells/animals, the DNA which repairs the site of interest comprises sequences that inactivate the DMD gene.

For making knock-in cells/animals, the DNA which repairs the site of interest comprises the sequence of an exogenous gene of interest, and eventually a selection marker, such as the neomycin resistance gene.

In a preferred embodiment, said targeting DNA construct is inserted in a vector.

The subject-matter of the present invention is also a method for making a dystrophin-deficient cell, comprising at least the step of:

(a) introducing into a cell, a meganuclease as defined above, so as to induce a double stranded cleavage at a site of interest of the DMD gene comprising a DNA recognition and cleavage site of said meganuclease, and thereby generate genetically modified DMD gene-deficient cell having repaired the double-strands break, by non-homologous end joining, and

(b) isolating the genetically modified DMD gene-deficient cell of step (a), by any appropriate mean.

The subject-matter of the present invention is also a method for making a DMD gene knock-out animal, comprising at least the step of:

(a) introducing into a pluripotent precursor cell or an embryo of an animal, a meganuclease, as defined above, so as to induce a double stranded cleavage at a site of interest of the DMD gene comprising a DNA recognition and cleavage site of said meganuclease, and thereby generate genetically modified precursor cell or embryo having repaired the double-strands break by non-homologous end joining,

(b) developing the genetically modified animal precursor cell or embryo of step (a) into a chimeric animal, and

(c) deriving a transgenic animal from a chimeric animal of step (b).

Preferably, step (b) comprises the introduction of the genetically modified precursor cell obtained in step (a), into blastocysts, so as to generate chimeric animals.

The cells which are modified may be any cells of interest as long as they contain the specific target site. For making knock-in/transgenic mice, the cells are pluripotent precursor cells such as embryo-derived stem (ES) cells, which are well-known in the art. For making recombinant human cell lines, the cells may advantageously be PerC6 (Fallaux et al., Hum. Gene Ther. 9, 1909-1917, 1998) or HEK293 (ATCC #CRL-1573) cells.

The animal is preferably a mammal, more preferably a laboratory rodent (mice, rat, guinea-pig), or a rabbit, a cow, pig, horse or goat.

Said meganuclease can be provided directly to the cell or through an expression vector comprising the polynucleotide sequence encoding said meganuclease and suitable for its expression in the used cell.

For making recombinant cell lines expressing an heterologous protein of interest, the targeting DNA comprises a sequence encoding the product of interest (protein or RNA), and eventually a marker gene, flanked by sequences upstream and downstream the cleavage site, as defined above, so as to generate genetically modified cells having integrated the exogenous sequence of interest in the DMD gene, by homologous recombination.

The sequence of interest may be any gene coding for a certain protein/peptide of interest, included but not limited to: reporter genes, receptors, signaling molecules, transcription factors, pharmaceutically active proteins and peptides, disease causing gene products and toxins. The sequence may also encode a RNA molecule of interest including for example an interfering RNA such as ShRNA, miRNA or siRNA, well-known in the art.

The expression of the exogenous sequence may be driven, either by the endogenous DMD gene promoter or by a heterologous promoter, preferably a ubiquitous or tissue specific promoter, either constitutive or inducible, as defined above. In addition, the expression of the sequence of interest may be conditional; the expression may be induced by a site-specific recombinase such as Cre or FLP (Akagi K, Sandig V, Vooijs M, Van der Valk M, Giovannini M, Strauss M, Berns A (May 1997). “Nucleic Acids Res. 25 (9): 1766-73.; Zhu X D, Sadowski P D (1995). J Biol Chem 270).

Thus, the sequence of interest is inserted in an appropriate cassette that may comprise an heterologous promoter operatively linked to said gene of interest and one or more functional sequences including but not limited to (selectable) marker genes, recombinase recognition sites, polyadenylation signals, splice acceptor sequences, introns, tag for protein detection and enhancers.

The subject matter of the present invention is also a kit for making DMD gene knock-out or knock-in cells/animals comprising at least a meganuclease and/or one expression vector, as defined above. Preferably, the kit further comprises a targeting DNA comprising a sequence that inactivates the DMD gene flanked by sequences sharing homologies with the region of the DMD gene surrounding the DNA cleavage site of said meganuclease. In addition, for making knock-in cells/animals, the kit includes also a vector comprising a sequence of interest to be introduced in the genome of said cells/animals and eventually a selectable marker gene, as defined above.

The subject-matter of the present invention is also the use of at least one meganuclease and/or one expression vector, as defined above, for the preparation of a medicament for preventing, improving or curing a pathological condition caused by a mutation in the DMD gene as defined above, in an individual in need thereof.

The use of the meganuclease may comprise at least the step of (a) inducing in somatic tissue(s) of the donor/individual a double stranded cleavage at a site of interest of the DMD gene comprising at least one recognition and cleavage site of said meganuclease by contacting said cleavage site with said meganuclease, and (b) introducing into said somatic tissue(s) a targeting DNA, wherein said targeting DNA comprises (1) DNA sharing homologies to the region surrounding the cleavage site and (2) DNA which repairs the DMD gene upon recombination between the targeting DNA and the chromosomal DNA, as defined above. The targeting DNA is introduced into the somatic tissues(s) under conditions appropriate for introduction of the targeting DNA into the site of interest.

According to the present invention, said double-stranded cleavage may be induced, ex vivo by introduction of said meganuclease into somatic cells from the diseased individual and then transplantation of the modified cells back into the diseased individual.

The subject-matter of the present invention is also a method for preventing, improving or curing a pathological condition caused by a mutation in the DMD gene, in an individual in need thereof, said method comprising at least the step of administering to said individual a composition as defined above, by any means. The meganuclease can be used either as a polypeptide or as a polynucleotide construct encoding said polypeptide. It is introduced into mouse cells, by any convenient means well-known to those in the art, which are appropriate for the particular cell type, alone or in association with either at least an appropriate vehicle or carrier and/or with the targeting DNA.

According to an advantageous embodiment of the uses according to the invention, the meganuclease (polypeptide) is associated with:
liposomes, polyethyleneimine (PEI); in such a case said association is administered and therefore introduced into somatic target cells.
membrane translocating peptides (Bonetta, The Scientist, 2002, 16, 38; Ford et al., Gene Ther., 2001, 8, 1-4; Wadia and Dowdy, Curr. Opin. Biotechnol., 2002, 13, 52-56); in such a case, the sequence of the variant/single-chain meganuclease is fused with the sequence of a membrane translocating peptide (fusion protein).

According to another advantageous embodiment of the uses according to the invention, the meganuclease (polynucleotide encoding said meganuclease) and/or the targeting DNA is inserted in a vector. Vectors comprising targeting DNA and/or nucleic acid encoding a meganuclease can be introduced into a cell by a variety of methods (e.g., injection, direct uptake, projectile bombardment, liposomes, electroporation). Meganucleases can be stably or transiently expressed into cells using expression vectors. Techniques of expression in eukaryotic cells are well known to those in the art. (See Current Protocols in Human Genetics: Chapter 12 “Vectors For Gene Therapy” & Chapter 13 “Delivery Systems for Gene Therapy”). Optionally, it may be preferable to incorporate a nuclear localization signal into the recombinant protein to be sure that it is expressed within the nucleus.

Once in a cell, the meganuclease and if present, the vector comprising targeting DNA and/or nucleic acid encoding a meganuclease are imported or translocated by the cell from the cytoplasm to the site of action in the nucleus.

Since meganucleases recognize a specific DNA sequence, any meganuclease developed in the context of human dystrophin gene therapy could be used in other contexts (other organisms, other loci, use in the context of a landing pad containing the site) unrelated with gene therapy of DMD in human as long as the site is present.

For purposes of therapy, the meganucleases and a pharmaceutically acceptable excipient are administered in a therapeutically effective amount. Such a combination is said to be administered in a “therapeutically effective amount” if the amount administered is physiologically significant. An agent is physiologically significant if its presence results in a detectable change in the physiology of the recipient. In the present context, an agent is physiologically significant if its presence results in a decrease in the severity of one or more symptoms of the targeted disease and in a genome correction of the lesion or abnormality. Vectors comprising targeting DNA and/or nucleic acid encoding a meganuclease can be introduced into a cell by a variety of methods (e.g., injection, direct uptake, projectile bombardment, liposomes, electroporation). Meganucleases can be stably or transiently expressed into cells using expression vectors. Techniques of expression in eukaryotic cells are well known to those in the art. (See Current Protocols in Human Genetics: Chapter 12 “Vectors For Gene Therapy” & Chapter 13 “Delivery Systems for Gene Therapy”).

In one embodiment of the uses according to the present invention, the meganuclease is substantially non-immunogenic, i.e., engender little or no adverse immunological response. A variety of methods for ameliorating or eliminating deleterious immunological reactions of this sort can be used in accordance with the invention. In a preferred embodiment, the meganuclease is substantially free of N-formyl methionine. Another way to avoid unwanted immunological reactions is to conjugate meganucleases to polyethylene glycol (“PEG”) or polypropylene glycol (“PPG”) (preferably of 500 to 20,000 daltons average molecular weight (MW)). Conjugation with PEG or PPG, as described by Davis et al. (U.S. Pat. No. 4,179,337) for example, can provide non-immunogenic, physiologically active, water soluble endonuclease conjugates with anti-viral activity. Similar methods also using a polyethylene—polypropylene glycol copolymer are described in Saifer et al. (U.S. Pat. No. 5,006,333).

The invention also concerns a prokaryotic or eukaryotic host cell which is modified by a polynucleotide or a vector as defined above, preferably an expression vector.

The invention also concerns a non-human transgenic animal or a transgenic plant, characterized in that all or a part of their cells are modified by a polynucleotide or a vector as defined above.

As used herein, a cell refers to a prokaryotic cell, such as a bacterial cell, or an eukaryotic cell, such as an animal, plant or yeast cell.

The subject-matter of the present invention is also the use of at least one meganuclease variant, as defined above, as a scaffold for making other meganucleases. For example, further rounds of mutagenesis and selection/screening can be performed on said variants, for the purpose of making novel meganucleases.

The different uses of the meganuclease and the methods of using said meganuclease according to the present invention include the use of the I-CreI variant, the single-chain chimeric meganuclease derived from said variant, the polynucleotide(s), vector, cell, transgenic plant or non-human transgenic mammal encoding said variant or single-chain chimeric meganuclease, as defined above.

The subject matter of the present invention is also an I-CreI variant having mutations at positions 28 to 40 and/or 44 to 77 of I-CreI that is useful for engineering the variants able to cleave a DNA target from the DMD gene, according to the present invention. In particular, the invention encompasses the I-CreI variants as defined in step (c) to (f) of the method for engineering I-CreI variants, as defined above, including the variants at positions 28, 30, 32, 33, 38 and 40, or 44, 68, 70, 75 and 77. The invention encompasses also the I-CreI variants as defined in step (g), (h), (i), (j), (k) and (l) of the method for engineering I-CreI variants, as defined above including the variants monomers constituting the single chain molecules of Table II to Table VII.

Single-chain chimeric meganucleases able to cleave a DNA target from the gene of interest are derived from the variants according to the invention by methods well-known in the art (Epinat et al., Nucleic Acids Res., 2003, 31, 2952-62; Chevalier et al., Mol. Cell., 2002, 10, 895-905; Steuer et al., Chembiochem., 2004, 5, 206-13; International PCT Applications WO 03/078619, WO 2004/031346 and WO 2009/095793). Any of such methods, may be applied for constructing single-chain chimeric meganucleases derived from the variants as defined in the present invention. In particular, the invention encompasses also the I-CreI variants defined in the tables II and IV.

The polynucleotide sequence(s) encoding the variant as defined in the present invention may be prepared by any method known by the man skilled in the art. For example, they are amplified from a cDNA template, by polymerase chain reaction with specific primers. Preferably the codons of said cDNA are chosen to favour the expression of said protein in the desired expression system.

The recombinant vector comprising said polynucleotides may be obtained and introduced in a host cell by the well-known recombinant DNA and genetic engineering techniques.

The I-CreI variant or single-chain derivative as defined in the present invention are produced by expressing the polypeptide(s) as defined above; preferably said polypeptide(s) are expressed or co-expressed (in the case of the variant only) in a host cell or a transgenic animal/plant modified by one expression vector or two expression vectors (in the case of the variant only), under conditions suitable for the expression or co-expression of the polypeptide(s), and the variant or single-chain derivative is recovered from the host cell culture or from the transgenic animal/plant.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, N.Y.: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

### DEFINITIONS

Amino acid residues in a polypeptide sequence are designated herein according to the one-letter code, in which, for example, Q means Gln or Glutamine residue, R means Arg or Arginine residue and D means Asp or Aspartic acid residue.
Amino acid substitution means the replacement of one amino acid residue with another, for instance the replacement of an Arginine residue with a Glutamine residue in a peptide sequence is an amino acid substitution.
Altered/enhanced/increased cleavage activity, refers to an increase in the detected level of meganuclease cleavage activity, see below, against a target DNA sequence by a second meganuclease in comparison to the activity of a first meganuclease against the target DNA sequence. Normally the second meganuclease is a variant of the first and comprise one or more substituted amino acid residues in comparison to the first meganuclease.
Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
by “meganuclease”, is intended an endonuclease having a double-stranded DNA target sequence of 12 to 45 bp. Said meganuclease is either a dimeric enzyme, wherein each domain is on a monomer or a monomeric enzyme comprising the two domains on a single polypeptide.
by “meganuclease domain” is intended the region which interacts with one half of the DNA target of a meganuclease and is able to associate with the other domain of the same meganuclease which interacts with the other half of the DNA target to form a functional meganuclease able to cleave said DNA target.
by “meganuclease variant” or “variant” it is intended a meganuclease obtained by replacement of at least one residue in the amino acid sequence of the parent meganuclease with a different amino acid.
by “peptide linker” it is intended to mean a peptide sequence of at least 10 and preferably at least 17 amino acids which links the C-terminal amino acid residue of the first monomer to the N-terminal residue of the second monomer and which allows the two variant monomers to adopt the correct conformation for activity and which does not alter the specificity of either of the monomers for their targets.
by “subdomain” it is intended the region of a LAGLIDADG homing endonuclease core domain which interacts with a distinct part of a homing endonuclease DNA target half-site.
by “targeting DNA construct/minimal repair matrix/repair matrix” it is intended to mean a DNA construct comprising a first and second portions which are homologous to regions 5′ and 3′ of the DNA target in situ. The DNA construct also comprises a third portion positioned between the first and second portion which comprise some homology with the corresponding DNA sequence in situ or alternatively comprise no homology with the regions 5′ and 3′ of the DNA target in situ. Following cleavage of the DNA target, a homologous recombination event is stimulated between the genome containing the dystrophin gene or part of the dystrophin gene and the repair matrix, wherein the genomic sequence containing the DNA target is replaced by the third portion of the repair matrix and a variable part of the first and second portions of the repair matrix.
by “functional variant” is intended a variant which is able to cleave a DNA target sequence, preferably said target is a new target which is not cleaved by the parent meganuclease. For example, such variants have amino acid variation at positions contacting the DNA target sequence or interacting directly or indirectly with said DNA target.
by “selection or selecting” it is intended to mean the isolation of one or more meganuclease variants based upon an observed specified phenotype, for instance altered cleavage activity. This selection can be of the variant in a peptide form upon which the observation is made or alternatively the selection can be of a nucleotide coding for selected meganuclease variant.
by “screening” it is intended to mean the sequential or simultaneous selection of one or more meganuclease variant (s) which exhibits a specified phenotype such as altered cleavage activity.
by “derived from” it is intended to mean a meganuclease variant which is created from a parent meganuclease and hence the peptide sequence of the meganuclease variant is related to (primary sequence level) but derived from (mutations) the sequence peptide sequence of the parent meganuclease.
by “I-CreI” is intended the wild-type I-CreI having the sequence of pdb accession code 1g9y, corresponding to the sequence SEQ ID NO: 1 in the sequence listing.
by “I-CreI variant with novel specificity” is intended a variant having a pattern of cleaved targets different from that of the parent meganuclease. The terms “novel specificity”, “modified specificity”, “novel cleavage specificity”, “novel substrate specificity” which are equivalent and used indifferently, refer to the specificity of the variant towards the nucleotides of the DNA target sequence. In the present patent application all the I-CreI variants described comprise an additional Alanine after the first Methionine of the wild type I-CreI sequence (SEQ ID NO: 1). These variants also comprise two additional Alanine residues and an Aspartic Acid residue after the final Proline of the wild type I-CreI sequence. These additional residues do not affect the properties of the enzyme and to avoid confusion these additional residues do not affect the numeration of the residues in I-CreI or a variant referred in the present patent application, as these references exclusively refer to residues of the wild type I-CreI enzyme (SEQ ID NO: 1) as present in the variant, so for instance residue 2 of I-CreI is in fact residue 3 of a variant which comprises an additional Alanine after the first Methionine.
by “I-CreI site” is intended a 22 to 24 bp double-stranded DNA sequence which is cleaved by I-CreI. I-CreI sites include the wild-type non-palindromic I-CreI homing site and the derived palindromic sequences such as the sequence 5′-t₋₁₂c₋₁₁a₋₁₀a₋₉a₋₈a₋₇c₋₆g₋₅t₋₄c₋₃g₋₂t₋₁a₊₁c₊₂g₊₃a₊₄c₊₅g₊₆t₊₇t₊₈t₊₉t₊₁₀g₊₁₁a₊₁₂ (SEQ ID NO: 2), also called C1221 (FIGS. 4, 6, 8, 10, 12 and 14).
by “domain” or “core domain” is intended the “LAGLIDADG homing endonuclease core domain” which is the characteristic α₁β₁β₂α₂β₃β₄α₃ fold of the homing endonucleases of the LAGLIDADG family, corresponding to a sequence of about one hundred amino acid residues. Said domain comprises four beta-strands (β₁β₂β₃β₄) folded in an anti-parallel beta-sheet which interacts with one half of the DNA target. This domain is able to associate with another LAGLIDADG homing endonuclease core domain which interacts with the other half of the DNA target to form a functional endonuclease able to cleave said DNA target. For example, in the case of the dimeric homing endonuclease I-CreI (163 amino acids), the LAGLIDADG homing endonuclease core domain corresponds to the residues 6 to 94.
by “subdomain” is intended the region of a LAGLIDADG homing endonuclease core domain which interacts with a distinct part of a homing endonuclease DNA target half-site.
by “chimeric DNA target” or “hybrid DNA target” it is intended the fusion of a different half of two parent meganuclease target sequences. In addition at least one half of said target may comprise the combination of nucleotides which are bound by at least two separate subdomains (combined DNA target).
by “beta-hairpin” is intended two consecutive beta-strands of the antiparallel beta-sheet of a LAGLIDADG homing endonuclease core domain (β₁β₂ or, β₃β₄) which are connected by a loop or a turn,
by “single-chain meganuclease”, “single-chain chimeric meganuclease”, “single-chain meganuclease derivative”, “single-chain chimeric meganuclease derivative” or “single-chain derivative” is intended a meganuclease comprising two LAGLIDADG homing endonuclease domains or core domains linked by a peptidic spacer. The single-chain meganuclease is able to cleave a chimeric DNA target sequence comprising one different half of each parent meganuclease target sequence.
by “DNA target”, “DNA target sequence”, “target sequence”, “target-site”, “target”, “site”, “site of interest”, “recognition site”, “recognition sequence”, “homing recognition site”, “homing site”, “cleavage site” is intended a 20 to 24 bp double-stranded palindromic, partially palindromic (pseudo-palindromic) or non-palindromic polynucleotide sequence that is recognized and cleaved by a LAGLIDADG homing endonuclease such as I-CreI, or a variant, or a single-chain chimeric meganuclease derived from I-CreI. These terms refer to a distinct DNA location, preferably a genomic location, at which a double stranded break (cleavage) is to be induced by the meganuclease. The DNA target is defined by the 5′ to 3′ sequence of one strand of the double-stranded polynucleotide, as indicate above for C1221. Cleavage of the DNA target occurs at the nucleotides at positions +2 and −2, respectively for the sense and the antisense strand. Unless otherwise indicated, the position at which cleavage of the DNA target by an I-Cre I meganuclease variant occurs, corresponds to the cleavage site on the sense strand of the DNA target.
by “DNA target half-site”, “half cleavage site” or half-site” is intended the portion of the DNA target which is bound by each LAGLIDADG homing endonuclease core domain.
by “chimeric DNA target” or “hybrid DNA target” is intended the fusion of different halves of two parent meganuclease target sequences. In addition at least one half of said target may comprise the combination of nucleotides which are bound by at least two separate subdomains (combined DNA target).
by “DMD gene” is intended a dystrophin gene (DMD), preferably the DMD gene of a vertebrate, more preferably the DMD gene of a mammal such as human. DMD gene sequences are available in sequence databases, such as the NCBI/GenBank database. This gene has been described in databanks as human dystrophin gene (DMD) NCBI NC_{—}000023.
by “DNA target sequence from the DMD gene”, “genomic DNA target sequence”, “genomic DNA cleavage site”, “genomic DNA target” or “genomic target” is intended a 22 to 24 bp sequence of the DMD gene as defined above, which is recognized and cleaved by a meganuclease variant or a single-chain chimeric meganuclease derivative.
by “parent meganuclease” it is intended to mean a wild type meganuclease or a variant of such a wild type meganuclease with identical properties or alternatively a meganuclease with some altered characteristic in comparison to a wild type version of the same meganuclease. In the present invention the parent meganuclease can refer to the initial meganuclease from which the first series of variants are derived in step (a) or the meganuclease from which the second series of variants are derived in step (b), or the meganuclease from which the third series of variants are derived in step (k).
by “vector” is intended a nucleic acid molecule capable of transporting another nucleic acid to which it has been linked.
by “homologous” is intended a sequence with enough identity to another one to lead to homologous recombination between sequences, more particularly having at least 95% identity, preferably 97% identity and more preferably 99%.
“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting.
by “mutation” is intended the substitution, deletion, insertion of one or more nucleotides/amino acids in a polynucleotide (cDNA, gene) or a polypeptide sequence. Said mutation can affect the coding sequence of a gene or its regulatory sequence. It may also affect the structure of the genomic sequence or the structure/stability of the encoded mRNA.
“gene of interest” or “GOI” refers to any nucleotide sequence encoding a known or putative gene product.
As used herein, the term “locus” is the specific physical location of a DNA sequence (e.g. of a gene) on a chromosome. The term “locus” usually refers to the specific physical location of an endonuclease's target sequence on a chromosome. Such a locus, which comprises a target sequence that is recognized and cleaved by an endonuclease according to the invention, is referred to as “locus according to the invention”.
by “safe harbor” locus of the genome of a cell, tissue or individual, is intended a gene locus wherein a transgene could be safely inserted, the disruption or deletion of said locus consecutively to the insertion not modifying expression of genes located outside of said locus, and/or the cellular proliferation and/or the growth rate of the cell, tissue or individual.
As used herein, the term “transgene” refers to a sequence encoding a polypeptide. Preferably, the polypeptide encoded by the transgene is either not expressed, or expressed but not biologically active, in the cell, tissue or individual in which the transgene is inserted. Most preferably, the transgene encodes a therapeutic polypeptide useful for the treatment of an individual.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLES

### Example 1

### Engineering Meganucleases Targeting the DMD21 Locus

a) Construction of Variants Targeting the DMD21 Locus

DMD21 is an example of a target for which meganuclease variants have been generated. The DMD21 target sequence (GA-AAC-CT-CAA-GTAC-CAA-AT-GTA-AA, SEQ ID NO: 4) is located at positions 993350-993373 in 3′ of exon 38 of DMD gene, within intron 38.

The DMD21 sequence is partially a combination of the 10AAC_P (SEQ ID NO: 5), 5CAA_P (SEQ ID NO: 6), 10TAC_P (SEQ ID NO: 7) and 5TTG_P (SEQ ID NO: 8) target sequences which are shown on FIG. 4. These sequences are cleaved by mega-nucleases obtained as described in International PCT applications WO 2006/097784 and WO 2006/097853, Arnould et al. (J. Mol. Biol., 2006, 355, 443-458) and Smith et al. (Nucleic Acids Res., 2006).

Two palindromic targets, DMD21.3 and DMD21.4, were derived from DMD21 (FIG. 4). Since DMD21.3 and DMD21.4 are palindromic, they are be cleaved by homodimeric proteins. Therefore, homodimeric I-CreI variants cleaving either the DMD21.3 palindromic target sequence of SEQ ID NO: 9 or the DMD21.4 palindromic target sequence of SEQ ID NO: 10 were constructed using methods derived from those described in Chames et al. (Nucleic Acids Res., 2005, 33, e178), Arnould et al. (J. Mol. Biol., 2006, 355, 443-458), Smith et al. (Nucleic Acids Res., 2006, 34, e149) and Arnould et al. (Arnould et al. J Mol Biol. 2007 371:49-65).

Single chain obligate heterodimer constructs were generated for the I-CreI variants able to cleave the DMD21 target sequences when forming heterodimers. These single chain constructs were engineered using the linker RM2 (AAGGSDKYNQALSKYNQALSKYNQALSGGGGS) (SEQ ID NO: 61). During this design step, mutations K7E, K96E were introduced into the mutant cleaving DMD21.3 (monomer 1) and mutations E8K, G19S, E61R into the mutant cleaving DMD21.4 (monomer 2) to create the single chain molecules: monomer1 (K7E K96E)-RM2-monomer2(E8K G19S E61R) that is called SCOH-DMD21 (Table II).

Four additional amino-acid substitutions have been found in previous studies to enhance the activity of I-CreI derivatives: these mutations correspond to the replacement of Phenylalanine 54 with Leucine (F54L), Glutamic acid 80 with Lysine (E80K), Valine 105 with Alanine (V105A) and Isoleucine 132 with Valine (I132V). Some combinations were introduced into the coding sequence of N-terminal and C-terminal protein fragment, and some of the resulting proteins were assayed for their ability to induce cleavage of the DMD21 target.

TABLE II
example of SCOH-DMD21 useful for DMD21 targeting

| pCLS SCOH | Nterminal mutations in Single | Cterminal mutations in Single | Cleavage | SEQ ID |
|---|---|---|---|---|
| DMD21 | Chains (SC) | Chains (SC) | in CHO | NO: |
| pCLS2872 | 7E30R40E44T46G68T70S73M7 | 8K19S28R33A38Y40Q61R70S | + | 62 |
| | 5A77R80K96E154N | 77K | | |
| pCLS2873 | 7E30R40E44T46G68T70S73M7 | 8K19S28R33A38Y40Q61R70S | + | 63 |
| | 5A77R80K96E132V154N | 77K | | |
| pCLS2874 | 7E30R40E44T46G68T70S73M7 | 8K19S28R33A38Y40Q61R70S | + | 64 |
| | 5A77R80K96E132V154N | 77K132V | | |
| pCLS2875 | 7E30R40E44T46G68T70S73M7 | 8K19S28R33A38Y40Q61R70S | + | 65 |
| | 5A77R80K96E154N | 77K132V | | |
| pCLS3385 | 7E30R40E44T46G68T70S73M7 | 8K19S28R33A38Y40Q61R70S | + | 66 |
| | 5A77R80K96E105A132V154N | 77K132V | | |
| pCLS3387 | 7E30R40E44T46G68T70S73M7 | 8K19S28R33A38Y40Q61R66H | + | 67 |
| | 5A77R80K96E132V154N | 70S77K132V | | |
| pCLS3388 | 7E30R40E44T46G68T70S73M7 | 8K19S28R33A38Y40Q61R70S | + | 68 |
| | 5A77R80K96E105A132V154N | 77K80K105A132V | | |
| pCLS5353 | 7E30R40E44T46G68T70S73M | 8K19S28R33A38Y40Q44R61R | + | 116 |
| | 75A77R80K96E132V154N | 68Y70S132V | | |
| pCLS5354 | 7E19S30R40E44T46G68T70S | 8K28R33A38Y40Q44R61R68Y | + | 117 |
| | 73M75A77R80K96E132V154N | 70S132V | | |
| pCLS5355 | 7E30R40E44T46G50R68T70S | 8K19S28R33A38Y40Q44R61R | + | 118 |
| | 73M75A77R96E132V | 68Y70S132V | | |
| pCLS5356 | 7E19S30R40E44T46G50R68T | 8K28R33A38Y40Q44R61R68Y | + | 119 |
| | 70S73M75A77R96E132V | 70S132V | | |

b) Validation of Some SCOH-DMD21 Variants in a Mammalian Cells Extrachromosomal Assay.

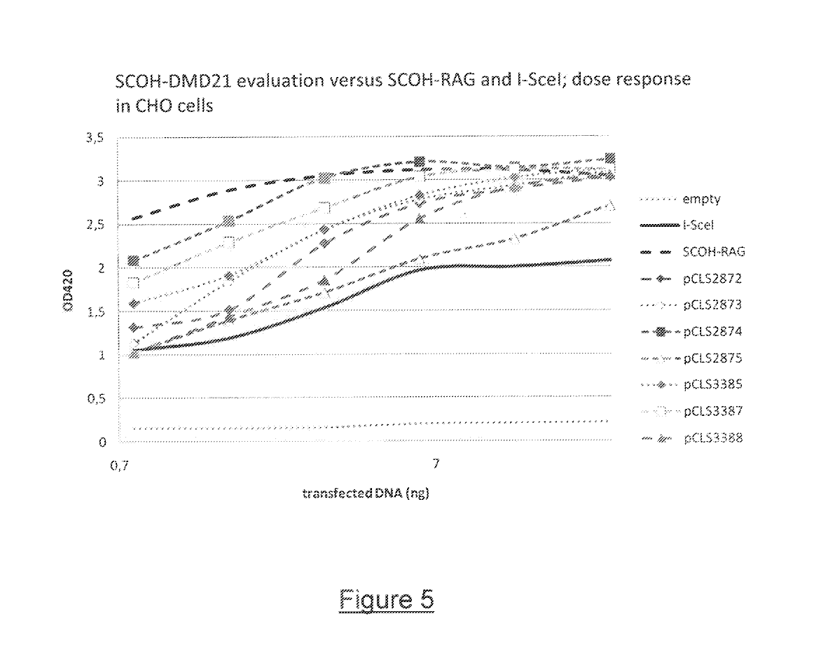

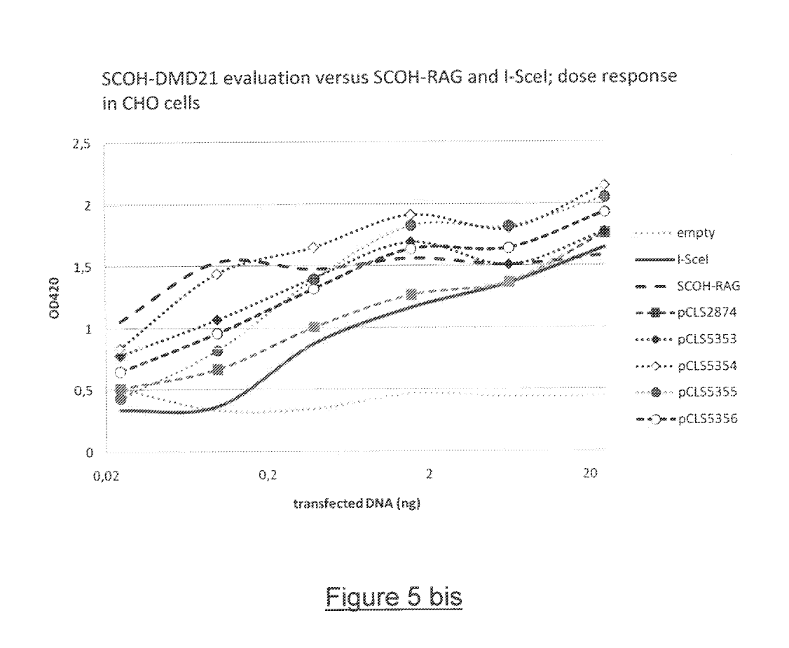

The activity of the single chain molecules against the DMD21 target was monitored using the described CHO assay along with our internal control SCOH-RAG and I-Sce I meganucleases. All comparisons were done from 0.78 to 25 ng transfected variant DNA (FIG. 5 and FIG. 5 bis). All the single molecules displayed DMD21 target cleavage activity in CHO assay as listed in Table II. Variants shared specific behaviour upon assayed dose depending on the mutation profile they bear (FIG. 5). For example, pCLS2874 has a similar profile than our standard control SCOH-RAG (pCLS2222). Its activity reaches the maxima at low DNA quantity transfected. All of the variants described are strongly active and can be used for targeting genes into the DMD21 locus.

### Example 2

### Engineering Meganucleases Targeting the DMD24 Locus

a) Construction of Variants Targeting the DMD24 Locus

DMD24 is an example of a target for which meganuclease variants have been generated. The DMD24 target sequence (TT-TAC-CT-ATT-TTAA-GTC-AG-ATA-CA, SEQ ID NO: 11) is located at positions 995930-995953 in 3′ of exon 39 of DMD gene, within intron 39.

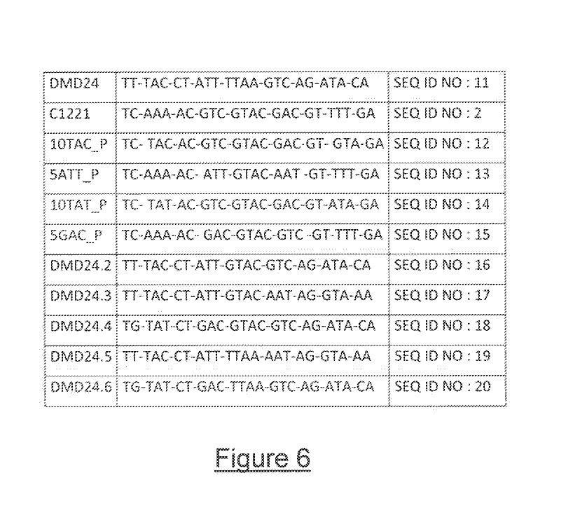

The DMD24 sequence is partially a combination of the 10TAC_P (SEQ ID NO: 12), 5ATT_P (SEQ ID NO: 13), 10TAT_P (SEQ ID NO: 14) and 5GAC_P (SEQ ID NO: 15) target sequences which are shown on FIG. 6. These sequences are cleaved by mega-nucleases obtained as described in International PCT applications WO 2006/097784 and WO 2006/097853, Arnould et al. (J. Mol. Biol., 2006, 355, 443-458) and Smith et al. (Nucleic Acids Res., 2006).

Two palindromic targets, DMD24.3 and DMD24.4, and two pseudo palindromic targets, DMD24.5 and DMD24.6, were derived from DMD24 and DMD24.2 (FIG. 6). Since DMD24.3 and DMD24.4 are palindromic, they are be cleaved by homodimeric proteins. Therefore, homodimeric I-CreI variants cleaving either the DMD24.3 palindromic target sequence of SEQ ID NO: 17 or the DMD24.4 palindromic target sequence of SEQ ID NO: 18 were constructed using methods derived from those described in Chames et al. (Nucleic Acids Res., 2005, 33, e178), Arnould et al. (J. Mol. Biol., 2006, 355, 443-458), Smith et al. (Nucleic Acids Res., 2006, 34, e149) and Arnould et al. (Arnould et al. J Mol. Biol. 2007 371:49-65).

Single chain obligate heterodimer constructs were generated for the I-CreI variants able to cleave the DMD24 target sequences when forming heterodimers. These single chain constructs were engineered using the linker RM2 (AAGGSDKYNQALSKYNQALSKYNQALSGGGGS) (SEQ ID NO: 61). During this design step, mutations K7E, K96E were introduced into the mutant cleaving DMD24.3 (monomer 1) and mutations E8K, G19S, E61R into the mutant cleaving DMD24.4 (monomer 2) to create the single chain molecules: monomer1 (K7E K96E)-RM2-monomer2(E8K G19S E61R) that is called SCOH-DMD24 (Table III).

Four additional amino-acid substitutions have been found in previous studies to enhance the activity of I-CreI derivatives: these mutations correspond to the replacement of Phenylalanine 54 with Leucine (F54L), Glutamic acid 80 with Lysine (E80K), Valine 105 with Alanine (V105A) and Isoleucine 132 with Valine (I132V). Some combinations were introduced into the coding sequence of N-terminal and C-terminal protein fragment, and some of the resulting proteins were assayed for their ability to induce cleavage of the DMD24 target.

TABLE III
example of SCOH-DMD24 useful for DMD24 targeting

| SCOH- | Nterminal mutations in Single | Cterminal mutations in Single | Cleavage | SEQ ID |
|---|---|---|---|---|
| DMD24 | Chains (SC) | Chains (SC) | in CHO | NO: |
| pCLS3397 | 7E24V32G33C68A70S75N77R79G | 8K19S32H33C40A44Y61R68Y70 | + | 69 |
| | 96E132V153G80K | S75R77V105A132V | | |
| pCLS3399 | 7E24V32G33C68A70S75N77R79G | 8K19S32H33C40A44Y61R68Y70 | + | 70 |
| | 96E132V153G80K105A | S75R77V132V | | |
| pCLS3400 | 7E24V32G33C40C68A70S75N77R | 8K19S32H33C40A44Y61R68Y70 | + | 71 |
| | 96E | S75R77V | | |
| pCLS3401 | 7E24V32G33C40C68A70S75N77R | 8K19S32H33C40A44Y61R68Y70 | + | 72 |
| | 96E132V | S75R77V132V | | |
| pCLS3402 | 7E24V32G33C40C68A70S75N77R | 8K19S32H33C40A44Y61R68Y70 | + | 73 |
| | 80K96E132V | S75R77V105A132V | | |
| pCLS4713 | 7E24V32G33C40C68A70S75N77R | 8K19S32H33C40A44Y61R68Y77 | + | 74 |
| | 80K96E132V | V105A132V | | |
| pCLS3403 | 7E24V32G33C40C68A70S75N77R | 8K19S32H33C40A44Y61R68Y70 | + | 75 |
| | 80K96E105A132V | S75R77V80K105A132V | | |
| pCLS3404 | 7E24V32G33C40C68A70S75N77R | 8K19S32H33C40A44Y61R68Y70 | + | 76 |
| | 80K96E105A132V | S75R77V132V | | |
| pCLS4327 | 7E24V32G33C40C68A70S75N77R | 8K19S32H33C40A44Y61R68Y77 | + | 77 |
| | 80K96E105A132V | V132V | | |

b) Validation of Some SCOH-DMD24 Variants in a Mammalian Cells Extrachromosomal Assay.

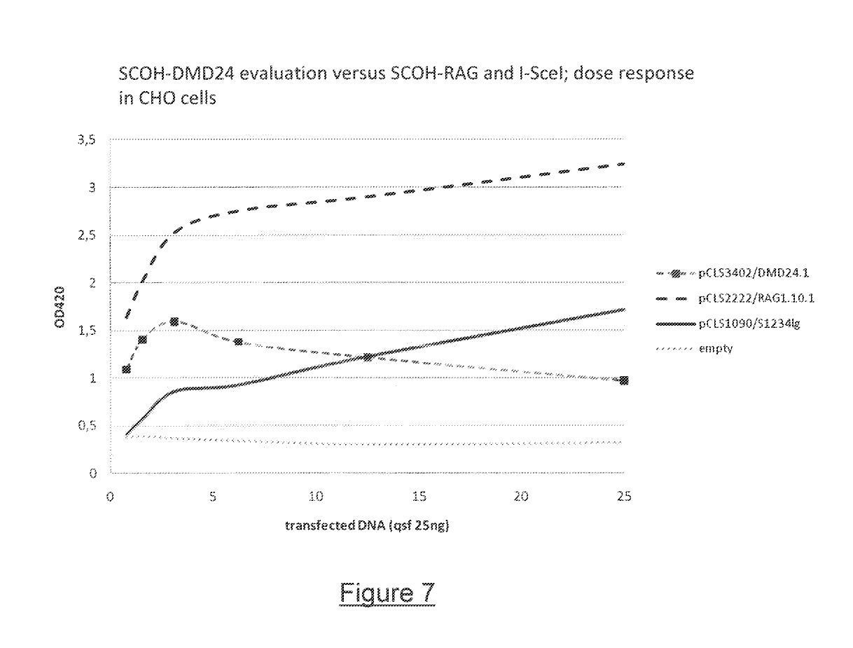

The activity of the single chain molecules against the DMD24 target was monitored using the described CHO assay along with our internal control SCOH-RAG and I-Sce I meganucleases. All comparisons were done from 0.78 to 25 ng transfected variant DNA (FIG. 7). All the single molecules displayed DMD24 target cleavage activity in CHO assay as listed in Table III. Variants shared specific behaviour upon assayed dose depending on the mutation profile they bear (FIG. 7). For example, pCLS3402 has a similar profile than our standard control SCOH-RAG (pCLS2222) at low doses, reaches and maxima and decrease with increasing DNA doses. All of the variants described are strongly active and can be used for targeting genes into the DMD24 locus.

### Example 3

### Engineering Meganucleases Targeting the DMD31 Locus

a) Construction of Variants Targeting the DMD31 Locus

DMD31 is an example of a target for which meganuclease variants have been generated. The DMD31 target sequence (AA-TGT-CT-GAT-GTTC-AAT-GT-GTT-GA, SEQ ID NO: 21) is located at positions 1125314-1125337 in 3′ of exon 44 of DMD gene, within intron 44.

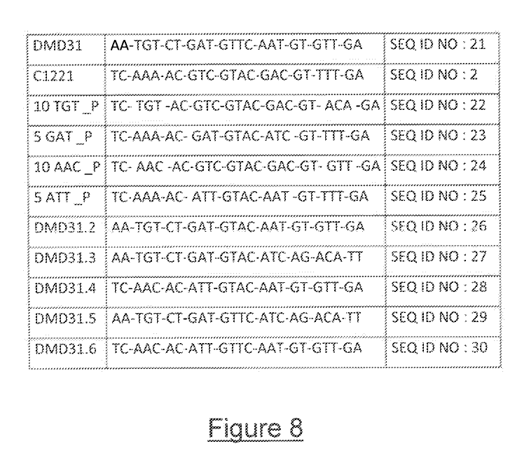

The DMD31 sequence is partially a combination of the 10 TGT_P (SEQ ID NO: 22), 5 GAT_P (SEQ ID NO: 23), 10 AAC_P (SEQ ID NO: 24) and 5 ATT_P (SEQ ID NO: 25) target sequences which are shown on FIG. 8. These sequences are cleaved by mega-nucleases obtained as described in International PCT applications WO 2006/097784 and WO 2006/097853, Arnould et al. (J. Mol. Biol., 2006, 355, 443-458) and Smith et al. (Nucleic Acids Res., 2006).

Two palindromic targets, DMD31.3 and DMD31.4, and two pseudo palindromic targets, DMD31.5 and DMD31.6, were derived from DMD31 and DMD31.2 (FIG. 8). Since DMD31.3 and DMD31.4 are palindromic, they are be cleaved by homodimeric proteins. Therefore, homodimeric I-CreI variants cleaving either the DMD31.3 palindromic target sequence of SEQ ID NO: 27 or the DMD31.4 palindromic target sequence of SEQ ID NO: 28 were constructed using methods derived from those described in Chames et al. (Nucleic Acids Res., 2005, 33, e178), Arnould et al. (J. Mol. Biol., 2006, 355, 443-458), Smith et al. (Nucleic Acids Res., 2006, 34, e149) and Arnould et al. (Arnould et al. J Mol Biol. 2007 371:49-65).

Single chain obligate heterodimer constructs were generated for the I-CreI variants able to cleave the DMD31 target sequences when forming heterodimers. These single chain constructs were engineered using the linker RM2 (AAGGSDKYNQALSKYNQALSKYNQALSGGGGS) (SEQ ID NO: 61). During this design step, mutations K7E, K96E were introduced into the mutant cleaving DMD31.3 (monomer 1) and mutations E8K, G19S, E61R into the mutant cleaving DMD31.4 (monomer 2) to create the single chain molecules: monomer1 (K7E K96E)-RM2-monomer2(E8K G19S E61R) that is called SCOH-DMD31 (Table IV). Four additional amino-acid substitutions have been found in previous studies to enhance the activity of I-CreI derivatives: these mutations correspond to the replacement of Phenylalanine 54 with Leucine (F54L), Glutamic acid 80 with Lysine (E80K), Valine 105 with Alanine (V105A) and Isoleucine 132 with Valine (I132V). Some combinations were introduced into the coding sequence of N-terminal and C-terminal protein fragment, and some of the resulting proteins were assayed for their ability to induce cleavage of the DMD31 target.

TABLE IV
example of SCOH-DMD31 useful for DMD31 targeting

| SCOH- | Nterminal mutations in | Cterminal mutations in | | SEQ ID |
|---|---|---|---|---|
| DMD31 | Single Chains (SC) | Single Chains (SC) | Cleavage in CHO | NO: |
| pCLS3627 | 7E30H32T33C38R44A50R70 | 8K19S24V33R40E44I60G61R | + | 78 |
| | S75Y77T96E132V | 70S75N77R129A132V156G | | |
| pCLS3628 | 7E30H32T33C38R44A50R70 | 8K19S24V33R40E44I60G61R | + | 79 |
| | S75Y77T80K96E132V | 70S75N77R105A129A132V1 | | |
| | | 56G | | |
| pCLS3629 | 7E30H32T33C38R44A50R70 | 8K19S24V33R40E44I60G61R | + | 80 |
| | S75Y77T80K96E105A132V | 70S75N77R80K105A129A13 | | |
| | | 2V156G | | |
| pCLS3630 | 7E30H32T33C38R44A70S75 | 8K19S33R40E44L59A61R62 | + | 81 |
| | Y77T80K96E | V70A75N77R80K129A156G | | |
| pCLS3631 | 7E30H32T33C38R44A70S75 | 8K19S33R40E44L59A61R62 | + | 82 |
| | Y77T80K96E132V | V70A75N77R80K129A132V1 | | |
| | | 56G | | |
| pCLS3632 | 7E30H32T33C38R44A70S75 | 8K19S33R40E44L59A61R62 | + | 83 |
| | Y77T80K96E105A132V | V70A75N77R80K105A129A | | |
| | | 132V156G | | |
| pCLS3633 | 7E30H32T33C38R44A70S75 | 8K19S33R40E44L59A61R62 | + | 84 |
| | Y77T80K96E105A132V | V70A75N77R80K129A132V1 | | |
| | | 56G | | |

b) Validation of Some SCOH-DMD31 Variants in a Mammalian Cells Extrachromosomal Assay.

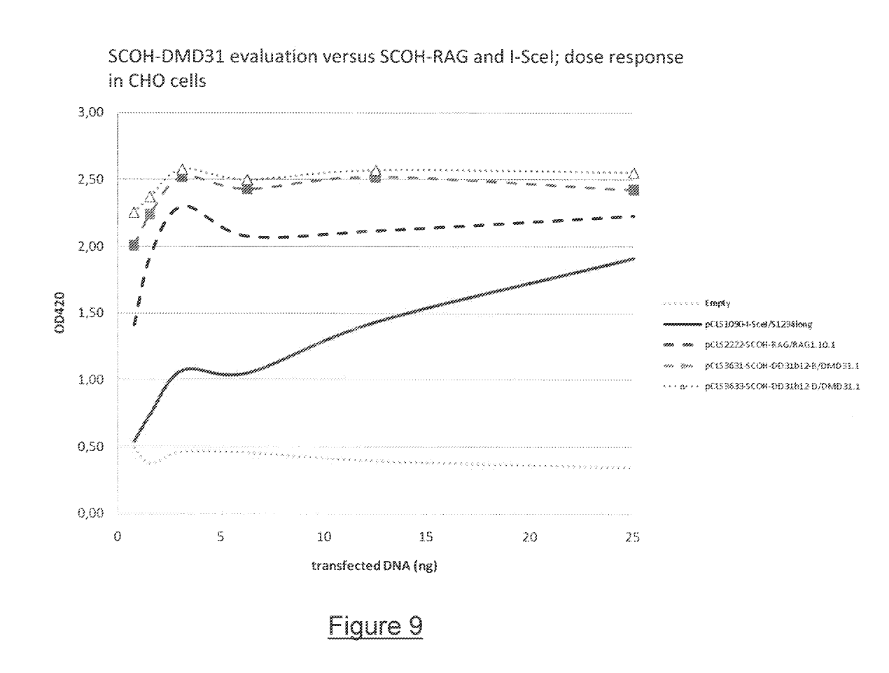

The activity of the single chain molecules against the DMD31 target was monitored using the described CHO assay along with our internal control SCOH-RAG and I-Sce I meganucleases. All comparisons were done from 0.78 to 25 ng transfected variant DNA (FIG. 9). All the single molecules displayed DMD31 target cleavage activity in CHO assay as listed in Table IV. Variants shared specific behaviour upon assayed dose depending on the mutation profile they bear (FIG. 9). For example, pCLS3631 and pCLS3633 have a similar profile, even higher activity, than our standard control SCOH-RAG (pCLS2222). They reach a maxima at very low DNA concentration. All of the variants described are strongly active and can be used for targeting genes into the DMD31 locus.

### Example 4

### Engineering Meganucleases Targeting the DMD33 Locus

a) Construction of Variants Targeting the DMD33 Locus

DMD33 is an example of a target for which meganuclease variants have been generated. The DMD33 target sequence (AA-ATC-CT-GCC-TTAA-AGT-AT-CTC-AT, SEQ ID NO: 31) is located at positions 1031834-1031857 in 3′ of exon 42 of DMD gene, within intron 42.

The DMD33 sequence is partially a combination of the 10 ATC_P (SEQ ID NO: 32), 5 GCC_P (SEQ ID NO: 33), 10 GAG_P (SEQ ID NO: 34) 5 ACT_P (SEQ ID NO: 35), target sequences which are shown on Figure. These sequences are cleaved by mega-nucleases obtained as described in International PCT applications WO 2006/097784 and WO 2006/097853, Arnould et al. (J. Mol. Biol., 2006, 355, 443-458) and Smith et al. (Nucleic Acids Res., 2006).

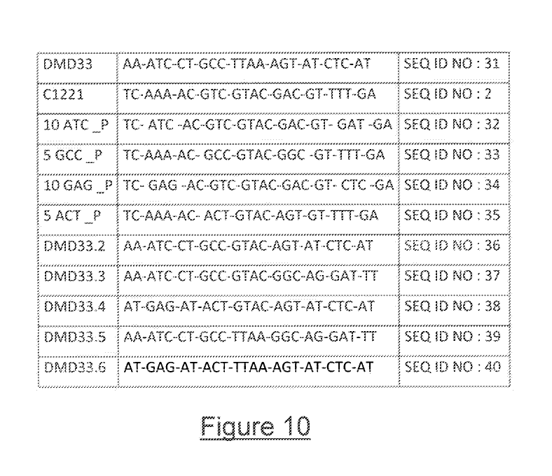

Two palindromic targets, DMD33.3 and DMD33.4, and two pseudo palindromic targets, DMD33.5 and DMD33.6, were derived from DMD33 and DMD33.2 (FIG. 10). Since DMD33.3 and DMD33.4 are palindromic, they are be cleaved by homodimeric proteins. Therefore, homodimeric I-CreI variants cleaving either the DMD33.3 palindromic target sequence of SEQ ID NO: 37 or the DMD33.4 palindromic target sequence of SEQ ID NO: 38 were constructed using methods derived from those described in Chames et al. (Nucleic Acids Res., 2005, 33, e178), Arnould et al. (J. Mol. Biol., 2006, 355, 443-458), Smith et al. (Nucleic Acids Res., 2006, 34, e149) and Arnould et al. (Arnould et al. J Mol Biol. 2007 371:49-65).

Single chain obligate heterodimer constructs were generated for the I-CreI variants able to cleave the DMD33 target sequences when forming heterodimers. These single chain constructs were engineered using the linker RM2 (AAGGSDKYNQALSKYNQALSKYNQALSGGGGS) (SEQ ID NO: 61). During this design step, mutations K7E, K96E were introduced into the mutant cleaving DMD33.3 (monomer 1) and mutations E8K, G19S, E61R into the mutant cleaving DMD33.4 (monomer 2) to create the single chain molecules: monomer1(K7E K96E)-RM2-monomer2(E8K G19S E61R) that is called SCOH-DMD33 (Table V).

Four additional amino-acid substitutions have been found in previous studies to enhance the activity of I-CreI derivatives: these mutations correspond to the replacement of Phenylalanine 54 with Leucine (F54L), Glutamic acid 80 with Lysine (E80K), Valine 105 with Alanine (V105A) and Isoleucine 132 with Valine (I132V). Some combinations were introduced into the coding sequence of N-terminal and C-terminal protein fragment, and some of the resulting proteins were assayed for their ability to induce cleavage of the DMD33 target.

TABLE V
example of SCOH-DMD33 useful for DMD33 targeting

| SCOH- | Nterminal mutations in Single | Cterminal mutations in | Cleavage in | SEQ ID |
|---|---|---|---|---|
| DMD33 | Chains (SC) | Single Chains (SC) | CHO | NO: |
| pCLS3326 | 7E30R32A33N40E44T68Y70S75 | 8K19S32G33H44K61R68Y70 | + | 85 |
| | K77Q80K96E132V | S75Y77N132V | | |
| pCLS3327 | 7E30R32A33N40E44T68Y70S75 | 8K19S32G33H44K61R68Y70 | + | 86 |
| | K77Q80K96E | S75Y77N132V | | |
| pCLS3328 | 7E30R32A33N40E44T68Y70S75 | 8K19S32G33H44K61R68Y70 | + | 87 |
| | K77Q80K96E132V | S75Y77N105A132V | | |
| pCLS3329 | 7E19S30R32A33N40E70S75R77 | 8K32G33H44K61R68Y70S77 | + | 88 |
| | T80K96E | K132V | | |
| pCLS3330 | 7E19S30R32A33N40E70S75R77 | 8K32G33H44K61R68Y70S77 | + | 89 |
| | T80K96E132V | K132V | | |
| pCLS3331 | 7E19S30R32A33N40E70S75R77 | 8K32G33H44K61R68Y70S77 | + | 90 |
| | T80K96E132V | K132V105A | | |
| pCLS3332 | 7E19S30R32A33N40E70S75R77 | 8K32G33H44K61R68Y70S77 | + | 91 |
| | T80K96E105A132V | K132V80K105A | | |
| pCLS3333 | 7E30R32A33N40E70S75R77T80 | 8K19S32G33H44K61R68Y70 | + | 92 |
| | K96E | S77K132V | | |
| pCLS3335 | 7E17A19S30R32A33N40E70S75 | 8K32G33H44K57E61R68Y70 | + | 93 |
| | R77T80K96E | S75Y77N132V | | |
| pCLS3336 | 7E17A19S30R32A33N40E70S75 | 8K32G33H44K57E61R68Y70 | + | 94 |
| | R77T80K96E132V | S75Y77N132V | | |
| pCLS3340 | 7E17A30R32A33N40E70S75R7 | 8K | + | 95 |
| | 7T80K96E132V | 19S32G33H44K57E61R68Y7 | | |
| | | 0S75Y77N132V | | |

b) Validation of Some SCOH-DMD33 Variants in a Mammalian Cells Extrachromosomal Assay.

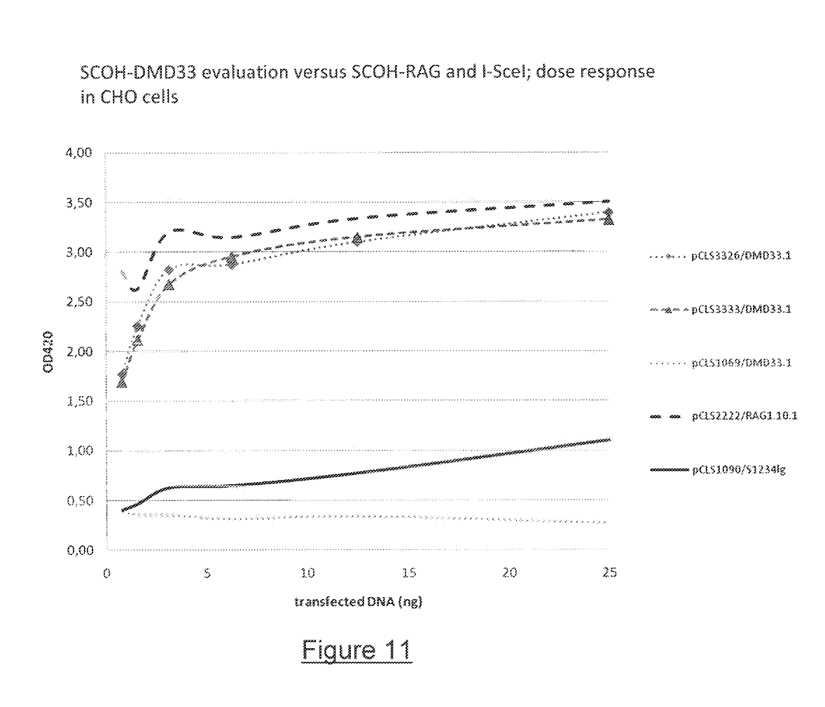

The activity of the single chain molecules against the DMD33 target was monitored using the described CHO assay along with our internal control SCOH-RAG and I-Sce I meganucleases. All comparisons were done from 0.78 to 25 ng transfected variant DNA (FIG. 11). All the single molecules displayed DMD33 target cleavage activity in CHO assay as listed in Table V. Variants shared specific behaviour upon assayed dose depending on the mutation profile they bear (FIG. 11). For example, pCLS3326 and pCLS3333 have a similar profile than our standard control SCOH-RAG (pCLS2222). They reach a maxima at very low DNA concentration. All of the variants described are strongly active and can be used for targeting genes into the DMD33 locus.

### Example 5

### Engineering Meganucleases Targeting the DMD35 Locus

a) Construction of Variants Targeting the DMD35 Locus

DMD35 is an example of a target for which meganuclease variants have been generated. The DMD35 target sequence (TC-TTT-AT-GTT-TTAA-AGT-AT-ATT-CC, SEQ ID NO: 41) is located at positions 1 561 221-1561244 in 5′ of exon 51 of DMD gene, within intron 50.

The DMD35 sequence is partially a combination of the 10 TTT_P (SEQ ID NO: 42), 5 GTT_P (SEQ ID NO: 43), 10 AAT_P (SEQ ID NO: 44) 5 ACT_P (SEQ ID NO: 45), target sequences which are shown on Figure. These sequences are cleaved by mega-nucleases obtained as described in International PCT applications WO 2006/097784 and WO 2006/097853, Arnould et al. (J. Mol. Biol., 2006, 355, 443-458) and Smith et al. (Nucleic Acids Res., 2006).

Two palindromic targets, DMD35.3 and DMD35.4, and two pseudo palindromic targets, DMD35.5 and DMD35.6, were derived from DMD35 and DMD35.2 (FIG. 12). Since DMD35.3 and DMD35.4 are palindromic, they are be cleaved by homodimeric proteins. Therefore, homodimeric I-CreI variants cleaving either the DMD35.3 palindromic target sequence of SEQ ID NO: 47 or the DMD35.4 palindromic target sequence of SEQ ID NO: 48 were constructed using methods derived from those described in Chames et al. (Nucleic Acids Res., 2005, 33, e178), Arnould et al. (J. Mol. Biol., 2006, 355, 443-458), Smith et al. (Nucleic Acids Res., 2006, 34, e149) and Arnould et al. (Arnould et al. J Mol Biol. 2007 371:49-65).

Single chain obligate heterodimer constructs were generated for the I-CreI variants able to cleave the DMD35 target sequences when forming heterodimers. These single chain constructs were engineered using either the linker RM2 (AAGGSDKYNQALSKYNQALSKYNQALSGGGGS) (SEQ ID NO: 61) for pCLS4901 (SEQ ID NO: 96), pCLS4902 (SEQ ID NO: 97), pCLS4903 (SEQ ID NO: 98) and pCLS4904 (SEQ ID NO: 99), either the linker BQY (GDSSVSNSEHIAPLSLPSSPPSVGS) (SEQ ID NO: 120) for pCLS6601 (SEQ ID NO: 121). During this design step, mutations K7E, K96E were introduced into the mutant cleaving DMD35.3 (monomer 1) and mutations E8K, G19S, E61R into the mutant cleaving DMD35.4 (monomer 2) to create the single chain molecules: monomer1(K7E K96E)-RM2-monomer2(E8K G19S E61R) that is called SCOH-DMD35 (Table VI).

Four additional amino-acid substitutions have been found in previous studies to enhance the activity of I-CreI derivatives: these mutations correspond to the replacement of Phenylalanine 54 with Leucine (F54L), Glutamic acid 80 with Lysine (E80K), Valine 105 with Alanine (V105A) and Isoleucine 132 with Valine (I132V). Some combinations were introduced into the coding sequence of N-terminal and C-terminal protein fragment, and some of the resulting proteins were assayed for their ability to induce cleavage of the DMD35 target.

TABLE VI
example of SCOH-DMD 35 useful for DMD35 targeting

| SCOH- | Nterminal mutations in | Cterminal mutations in Single | Cleavage in | SEQ ID |
|---|---|---|---|---|
| DMD35 | Single Chains (SC) | Chains (SC) | CHO | NO: |
| pCLS4901 | 7E33C38A54L70S75H77Y96 | 8K19S24V32K44R46A61R66H6 | + | 96 |
| | E132V | 8Y70S75Y77N115T132V | | |
| pCLS4902 | 7E33C38A54L70S75H77Y80 | 8K19S24V32K44R46A54L61R66 | + | 97 |
| | K96E 132V | H68Y70S75Y77N111R121R132 | | |
| | | V139R | | |
| pCLS4903 | 7E33C38A54L70S75H77Y80 | 8K19S24V32K44R46A61R66H6 | + | 98 |
| | K96E 132V | 8Y70S75Y77N81V132V | | |
| pCLS4904 | 7E33C38A54L70S75H77Y80 | 8K19S24V32K44R46A61R66H6 | + | 99 |
| | K96E 132V | 8Y70S75Y77N1158K T132V | | |
| pCLS6601 | 7E19S33C38A54L70S75H | 8K24V32K44R60E61R64A66H | + | 121 |
| | 77Y80K96E132V | 68Y70F75Y77N79C109T132V | | |

b) Validation of Some SCOH-DMD35 Variants in a Mammalian Cells Extrachromosomal Assay.

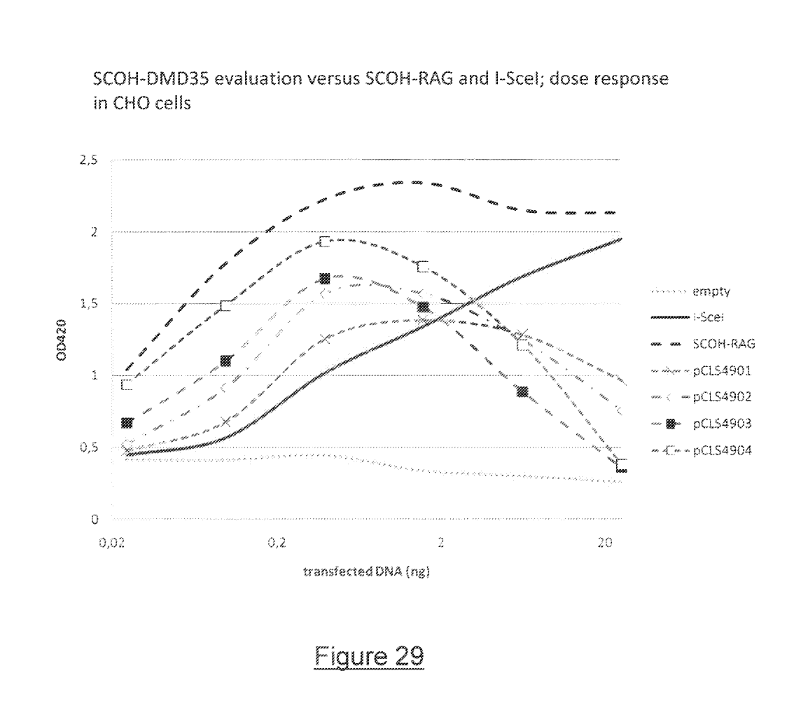

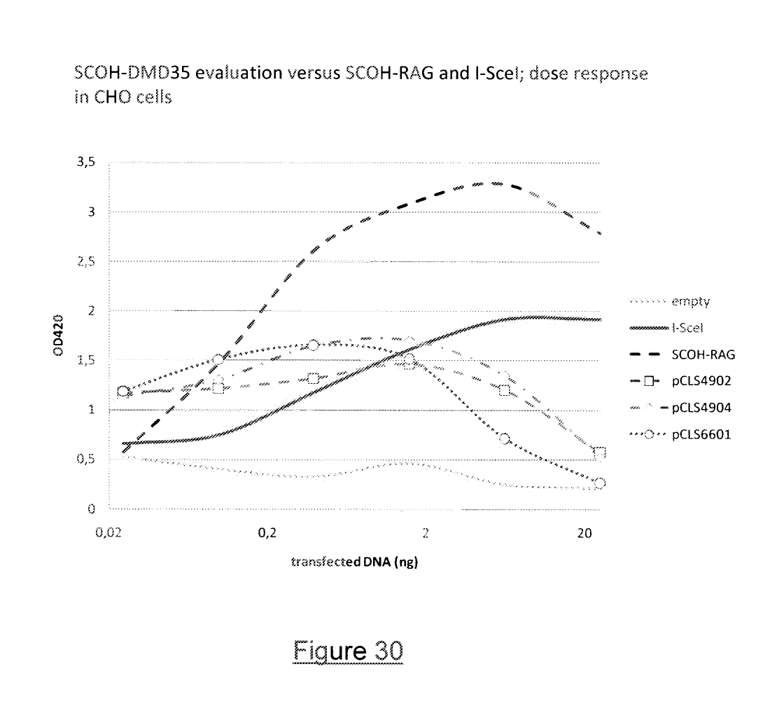

The activity of the single chain molecules against the DMD35 target was monitored using the described CHO assay along with our internal control SCOH-RAG and I-Sce I meganucleases. All comparisons were done from 0.78 to 25 ng transfected variant DNA (FIG. 29 and FIG. 30). All the single molecules displayed DMD35 target cleavage activity in CHO assay as listed in Table VI.

### Example 6

### Engineering Meganucleases Targeting the DMD37 Locus

a) Construction of Variants Targeting the DMD37 Locus

DMD37 is an example of a target for which meganuclease variants have been generated. The DMD37 target sequence (GA-ATC-CT-GTT-GTTC-ATC-AT-CCT-AG, SEQ ID NO: 51) is located at positions 1 659 873-1659896 in 5′ of exon 53 of DMD gene, within intron 52.

The DMD37 sequence is partially a combination of the 10 ATC_P (SEQ ID NO: 52), 5 GTT_P (SEQ ID NO: 53), 10 AGG_P (SEQ ID NO: 54) 5 GAT_P (SEQ ID NO: 55), target sequences which are shown on Figure. These sequences are cleaved by mega-nucleases obtained as described in International PCT applications WO 2006/097784 and WO 2006/097853, Arnould et al. (J. Mol. Biol., 2006, 355, 443-458) and Smith et al. (Nucleic Acids Res., 2006).

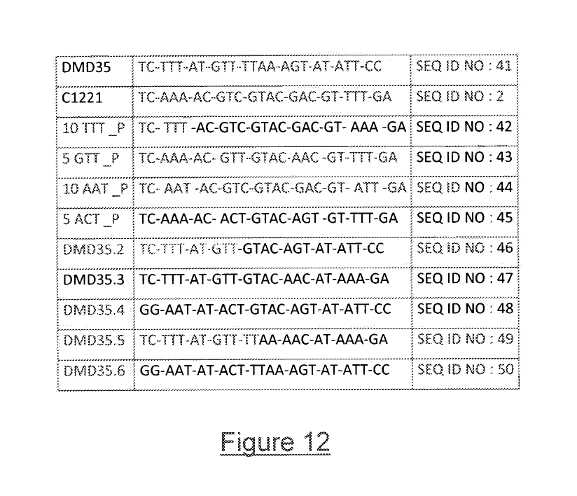

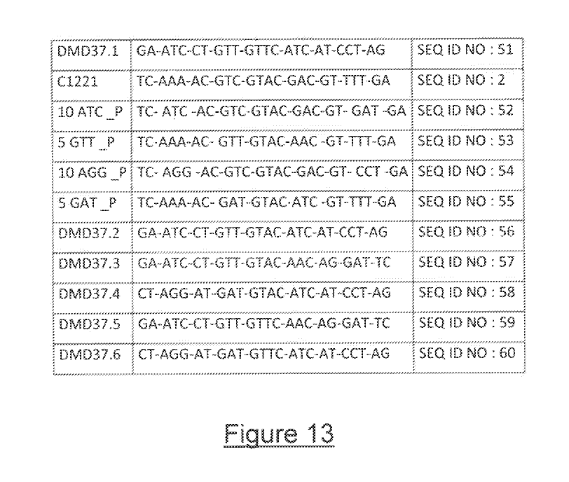

Two palindromic targets, DMD37.3 and DMD37.4, and two pseudo palindromic targets, DMD37.5 and DMD37.6, were derived from DMD37 and DMD37.2 (FIG. 13). Since DMD37.3 and DMD37.4 are palindromic, they are be cleaved by homodimeric proteins. Therefore, homodimeric I-CreI variants cleaving either the DMD37.3 palindromic target sequence of SEQ ID NO: 57 or the DMD37.4 palindromic target sequence of SEQ ID NO: 58 were constructed using methods derived from those described in Chames et al. (Nucleic Acids Res., 2005, 33, e178), Arnould et al. (J. Mol. Biol., 2006, 355, 443-458), Smith et al. (Nucleic Acids Res., 2006, 34, e149) and Arnould et al. (Arnould et al. J Mol. Biol. 2007 371:49-65).

Single chain obligate heterodimer constructs were generated for the I-CreI variants able to cleave the DMD37 target sequences when forming heterodimers. These single chain constructs were engineered using either the linker RM2 (AAGGSDKYNQALSKYNQALSKYNQALSGGGGS) (SEQ ID NO: 61) for pCLS4612 (SEQ ID NO: 122), pCLS4613 (SEQ ID NO: 123), pCLS4614 (SEQ ID NO: 124), pCLS7389 (SEQ ID NO: 127), pCLS7390 (SEQ ID NO: 128), pCLS7391 (SEQ ID NO: 129) and pCLS7392 (SEQ ID NO: 130), either the linker BQY (GDSSVSNSEHIAPLSLPSSPPSVGS) (SEQ ID NO: 120) for pCLS6602 (SEQ ID NO: 125) and pCLS6603 (SEQ ID NO: 126). During this design step, mutations K7E, K96E were introduced into the mutant cleaving DMD37.3 (monomer 1) and mutations E8K, G19S, E61R into the mutant cleaving DMD37.4 (monomer 2) to create the single chain molecules: monomer1 (K7E K96E)-RM2-monomer2(E8K G19S E61R) that is called SCOH-DMD37 (Table VII).

Four additional amino-acid substitutions have been found in previous studies to enhance the activity of I-CreI derivatives: these mutations correspond to the replacement of Phenylalanine 54 with Leucine (F54L), Glutamic acid 80 with Lysine (E80K), Valine 105 with Alanine (V105A) and Isoleucine 132 with Valine (I132V). Some combinations were introduced into the coding sequence of N-terminal and C-terminal protein fragment, and some of the resulting proteins were assayed for their ability to induce cleavage of the DMD37 target.

TABLE VII
example of SCOH-DMD37 useful for DMD37 targeting

| SCOH- | Nterminal mutations in | Cterminal mutations in | Cleavage in | SEQ ID |
|---|---|---|---|---|
| DMD37 | Single Chains (SC) | Single Chains (SC) | CHO | NO: |
| pCLS4606 | 7E16L30T40R54L70S75H77Y | 8K19S30G38R44T61R70Q | + | 100 |
| | 96E | 75Y96R | | |
| pCLS4607 | 7E16L30T40R54L70S75H77Y | 8K19S30G38R44T61R70Q | + | 101 |
| | 96E132V | 75Y96R132V | | |
| pCLS4608 | 7E16L30T40R54L70S75H77Y | 8K19S30G38R44T61R70Q | + | 102 |
| | 80K96E132V | 75Y96R132V | | |
| pCLS4609 | 7E19S16L30T40R54L70S75H | 8K30G38R39I44A61R70Q7 | + | 103 |
| | 77Y96E | 5N92H103D120G140K147S | | |
| pCLS4610 | 7E19S16L30T40R54L70S75H | 8K30G38R39I44A61R70Q7 | + | 104 |
| | 77Y96E | 5N92H103D120G132V140 | | |
| | | K147S | | |
| pCLS4611 | 7E19S16L30T40R54L70S75H | 8K30G38R39I44A61R70Q7 | + | 105 |
| | 77Y96E | 5N80K92H103D120G132V | | |
| | | 140K147S | | |
| pCLS4612 | 7E19S30T40R70S75H77Y | 8K30G38R44T61R70Q75Y | + | 122 |
| | 96E105A | 96R | | |
| pCLS4613 | 7E19S30T40R70S75H77Y | 8K30G38R44T61R70Q75Y | + | 123 |
| | 96E105A132V | 96R132V | | |
| pCLS4614 | 7E19S30T40R70S75H77Y | 8K30G38R44T61R70Q75Y | + | 124 |
| | 96E105A132V | 80K96R132V | | |
| pCLS6602 | 7E16L30T40R54L70S75H77Y | 8K19S30G38R44T61R70Q | + | 125 |
| | 96E132V | 75Y132V | | |
| pCLS6603 | 7E30T33T68A70S75N77R | 8K19S30G38R44T61R70Q | + | 126 |
| | 80K96E132V | 75Y96R132V | | |
| pCLS7389 | 7E16L30T40R54L70S75H77Y | 8K19S30G38R44T61R70Q | + | 127 |
| | 96E105A132V | 75Y96R132V | | |
| pCLS7390 | 7E16L30T40R54L70S75H77Y | 8K19S30G38R44T61R70Q | + | 128 |
| | 80K96E105A132V | 75Y96R132V | | |
| pCLS7391 | 7E30T40R70S75H77Y96E | 8K19S30G38R44T61R70Q | + | 129 |
| | 105A132V | 75Y96R132V | | |
| pCLS7392 | 7E30T40R70S75H77Y96E | 8K19S30G38R44T61R70Q | + | 130 |
| | 105A132V | 75Y80K96R132V | | |

b) Validation of Some SCOH-DMD37 Variants in a Mammalian Cells Extrachromosomal Assay.

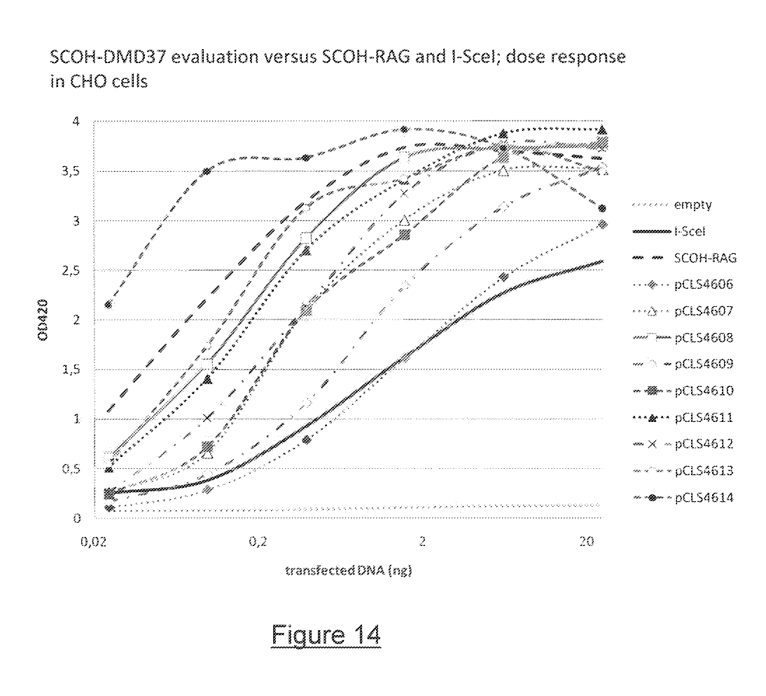

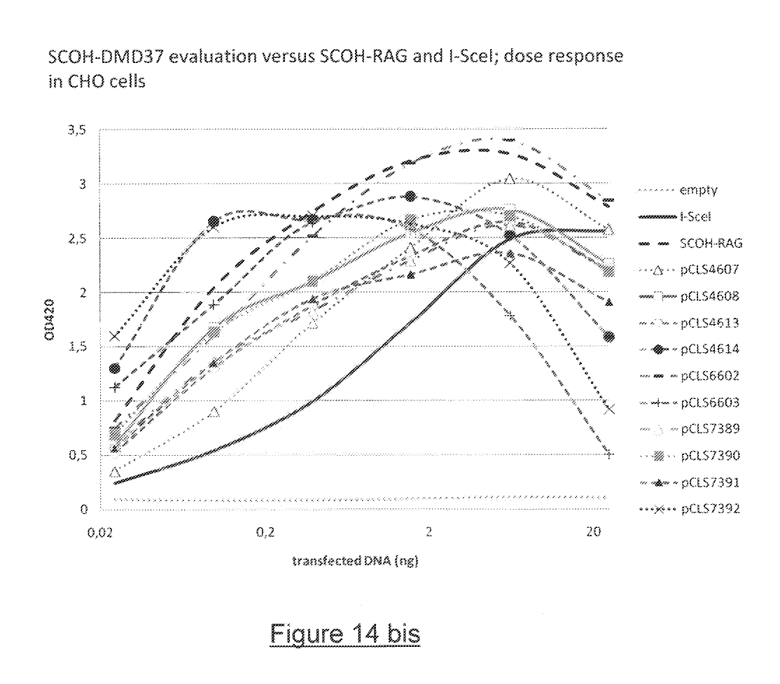

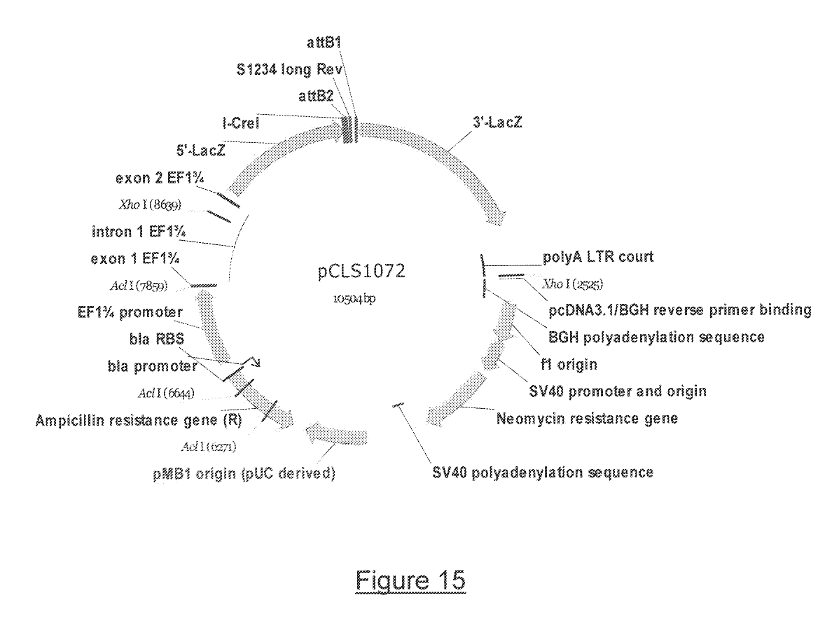

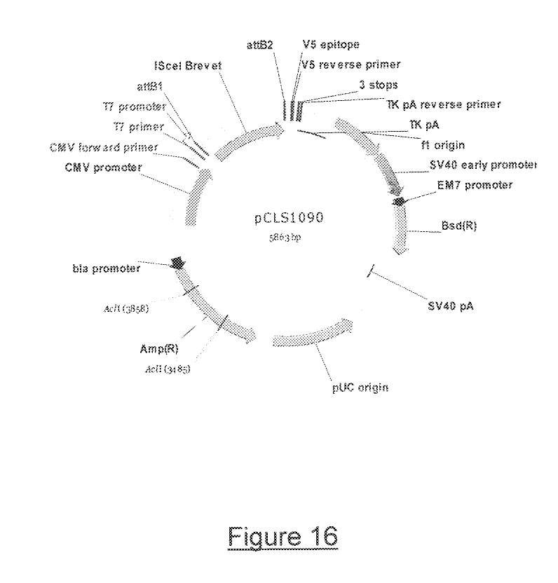

The activity of the single chain molecules against the DMD37 target was monitored using the described CHO assay along with our internal control SCOH-RAG and I-Sce I meganucleases. All comparisons were done from 0.78 to 25 ng transfected variant DNA (FIG. 14). All the single molecules displayed DMD37 target cleavage activity in CHO assay as listed in Table VII. Variants shared specific behaviour upon assayed dose depending on the mutation profile they bear (FIG. 14 and FIG. 14 bis). For example, pCLS4607 and pCLS4608 have a similar profile, even higher, than our standard control SCOH-RAG (pCLS2222). They reach a maxima at very low DNA concentration. All of the variants described are strongly active and can be used for targeting genes into the DMD37 locus.

### Example 7

### Cloning and Extrachromosomal Assay in Mammalian Cells

a) Cloning of DMD21, DMD24, DMD31, DMD33, DMD35, DMD37 Targets in a Vector for CHO Screen

The targets were cloned as follows using oligonucleotide corresponding to the target sequence flanked by gateway cloning sequence; the following oligonucleotides were ordered from PROLIGO.

These oligonucleotides have the following sequences:

DMD21:
(SEQ ID NO: 106)
5′- TGGCATACAAGTTTGAAACCTCAAGTACCAAATGTAAACAATCG
TCTGTCA -3′,
DMD24:
(SEQ ID NO: 107)
5′- TGGCATACAAGTTTTTTACCTATTTTAAGTCAGATACACAATCG
TCTGTCA -3′,
DMD31:
(SEQ ID NO: 108)
5′- TGGCATACAAGTTTAATGTCTGATGTTCAATGTGTTGACAATCG
TCTGTCA -3′,
DMD33:
(SEQ ID NO: 109)
5′- TGGCATACAAGTTTAAATCCTGCCTTAAAGTATCTCATCAATCG
TCTGTCA -3′,
DMD35:
(SEQ ID NO: 110)
5′- TGGCATACAAGTTTTCTTTATGTTTTAAAGTATATTCCCAATCG
TCTGTCA -3′,
DMD37:
(SEQ ID NO: 111)
5′- TGGCATACAAGTTTGAATCCTGTTGTTCATCATCCTAGCAATCG
TCTGTCA -3′

Double-stranded target DNA, generated by PCR amplification of the single stranded oligonucleotide, was cloned using the Gateway protocol (INVITROGEN) into CHO reporter vector (pCLS1058). Target was cloned and verified by sequencing (MILLEGEN).

b) Cloning of the Single Chain Molecules

A series of synthetic gene assembly was ordered to TopGene Technologies, MWG-EUROFINS. Synthetic genes coding for the different single chain variants targeting DMD were cloned in pCLS1853 using AscI and XhoI restriction sites.

c) Extrachromosomal Assay in Mammalian Cells

CHO K1 cells were transfected as described in example 1.2. 72 hours after transfection, culture medium was removed and 150 μl of lysis/revelation buffer for b-galactosidase liquid assay was added. After incubation at 37° C., OD was measured at 420 nm. The entire process is performed on an automated Velocity 11 BioCel platform. Per assay, 150 ng of target vector was cotransfected with an increasing quantity of variant DNA from 0.8 to 25 ng. The total amount of transfected DNA was completed to 175 ng (target DNA, variant DNA, carrier DNA) using an empty vector (pCLS0002).

Numerous modifications and variations on the present invention are possible in light of the above teachings. It is, therefore, to be understood that within the scope of the accompanying claims, the invention may be practiced otherwise than as specifically described herein.

### Example 8

### Meganuclease Activity at the DMD21 and DMD37 Loci: Example of Mutagenesis and Homologous Recombination

a) Meganuclease-Induced Mutagenesis Assay

The efficiency of the dedicated meganucleases to promote mutagenesis at their endogenous recognition site was evaluated by sequencing the DNA surrounding the meganuclease cleavage site after transfection of human 293H cells with, respectively, expression vectors bearing SCOH-DMD21 or SCOH-DMD37 genes without DNA repair matrix. Following the conditions described below, genomic DNA was extracted and DNA fragments bearing the targeted locus was amplified by PCR and submitted to 454 sequencing. The background was calculated using the sample conditions but an empty expression vector. PCR fragments carrying mutations were quantified and compared with the initial sequence. The percentage of PCR fragments carrying insertion or deletion at the meganuclease cleavage site was related to the mutagenesis induced by the meganuclease through NHEJ pathway in a cell population,

Materials and Methods

The human 293H cells (ATCC) were plated at a density of 1.2×10⁶ cells per 10 cm dish in complete medium (DMEM supplemented with 2 mM L-glutamine, penicillin (100 IU/ml), streptomycin (100 μg/ml), amphotericin B (Fongizone: 0.25 μg/ml, Invitrogen-Life Science) and 10% FBS. For this assay, 293H cells were co-transfected the following day with 3 μg of DMD21 or DMD37 meganuclease expressing vector using Lipofectamine 2000 transfection reagent (Invitrogen) according to the manufacturer's protocol.

Seven days post-transfection, genomic DNA was extracted. 200 ng of genomic DNA were used to amplify (PCR amplification) the endogenous locus surrounding the meganuclease cleavage site. PCR amplification is performed to obtain a fragment flanked by specific adaptor sequences [adaptor A: 5′-CCATCTCATCCCTGCGTGTCTCCGACTCAG-NNNN-3′ (SEQ ID NO: 131) and adaptor B, 5′-CCTATCCCCTGTGTGCCTTGGCAGTCTCAG-3′ (SEQ ID NO: 132)] provided by the company offering sequencing service (GATC Biotech AG, Germany) on the 454 sequencing system (454 Life Sciences). The primers sequences used for PCR amplification were DMD21_F: 5′-CCATCTCATCCCTGCGTGTCTCCGACTCAG-NNNN-AATTTCTAGAACTACACTAAAAAAGC-3′ (SEQ ID NO: 133) and DMD21_R: 5′-CCTATCCCCTGTGTGCCTTGGCAGTCTCAGAAACAACAAGTACAGTCTTCATTTTGG-3′ (SEQ ID NO: 134) and DMD37_F: 5′-CCATCTCATCCCTGCGTGTCTCCGACTCAG-NNNN-TCAACTGTTGCCTCCGGTTCTG-3′ (SEQ ID NO: 135) and DMD37_R: 5′-CCTATCCCCTGTGTGCCTTGGCAGTCTCAG-TGATGGGTGCTGAAGTGGCA-3′(SEQ ID NO: 136). Sequences specific to the locus are underlined. The sequence NNNN in primer F1 is a Barcode sequence (Tag) needed to link the sequence with a PCR product. The percentage of PCR fragments carrying insertion or deletion at the meganuclease cleavage site is related to the mutagenesis induced by the meganuclease through NHEJ pathway in a cell population, and therefore correlates with the meganuclease activity at its endogenous recognition site. 5000 to 10000 sequences were analyzed per conditions.

Results

Designed meganucleases targeting the DMD21 or the DMD37 sequences are able to promote InDel (Insertion/deletion) mutations in 1.4% and 1.0% (background 0.05% and 0.08%) (Table VIII).

TABLE VIII
Examples of InDel events and homologous recombination efficiencies
at the DMD21 and DMD37 loci using specific DMD21 and DMD37
meganucleases.

| | | | | RH frequencies |
|---|---|---|---|---|
| | | | InDel | corrected |
| | | InDel | background | by plating |
| Meganuclease | Plasmid | (%) | (%) | efficiency (%) |
| DMD21 | pCLS2874 | 1.4 | 0.05 | 8.7 |
| | (SEQ ID NO: 64) | | | |
| DMD37 | pCLS4607 | 1.0 | 0.08 | 4.0 |
| | (SEQ ID NO: | | | |
| | 101) | | | |

b) Meganuclease-Induced Gene Targeting Assay

Meganucleases were then evaluated for gene targeting at the endogenous locus DMD21 or DMD37. Following the method described below, expression vectors bearing the meganuclease gene and a DNA repair matrix were co-tranfected in 293H cells. A specific matrix was designed for DMD21 or DMD37 locus. After treatment, genomic DNA was extracted and targeted DNA matrix integration was monitored by specific PCR amplification.

Materials and Methods

The human 293H cells (ATCC) were plated at a density of 1.2×10⁶ cells per 10 cm dish in complete medium (DMEM supplemented with 2 mM L-glutamine, penicillin (100 IU/ml), streptomycin (100 μg/ml), amphotericin B (Fongizone: 0.25 μg/ml, Invitrogen-Life Science) and 10% FBS. For this assay, 293H cells were co-transfected the following day with 3 μg of DMD21 or DMD37 meganuclease expressing vector and 2 μg of respective DNA repair matrix using Lipofectamine 2000 transfection reagent (Invitrogen) according to the manufacturer's protocol.

The DNA repair matrix consists of a left and right arms corresponding to isogenic sequences of 1 kb located on both sides of the meganuclease recognition site. These two homology arms are separated by a heterologous fragment of 29 bp (sequence: AATTGCGGCCGCGGTCCGGCGCGCCTTAA, SEQ ID NO: 137). Two days post-transfection, cells were replated in 96 wells plate at a density of 10 cells per well. Two weeks later, DNA extraction was performed with the ZR-96 genomic DNA kit (Zymo research) according to the supplier's protocol. The detection of targeted DNA matrix integration at DMD21 locus was performed by specific PCR amplification using the primers DMD21_KI_F: 5′-AGGCCTCCATTCCTTTGAAGGAATTGG-3′ (SEQ ID NO: 138) and DMD21_KI_R: 5′-CCGGCGCGCCTTAAACTTGAGG-3′ (SEQ ID NO: 139); DMD21_KI_F is located outside the left homology arm of the integration matrix and DMD21_KI_R is located inside the heterologous fragment of said integration matrix. The detection of targeted DNA matrix integration at DMD37 locus was performed by specific PCR amplification using the primers DMD37_KI_F: 5′-TTAAGGCGCGCCGGACCGCGGC-3′ (SEQ ID NO: 140) and DMD37_KI_R: 5′-GCATCAGTTGCCTGGTATGTCTAGC-3′(SEQ ID NO: 141); DMD37_KI_F is located inside the heterologous fragment of the integration matrix and DMD37_KI_R is located outside the right homology arm of said integration matrix.

Results

Some results are shown in previous table VIII. The frequencies of targeted integrations using meganucleases designed for DMD21 and DMD37 sequences can respectively reach 8.7% and 4.0% with no selection pressure.

These results demonstrate that the meganucleases tailored for DMD21 or DMD37 sequences are active at their endogenous locus and can promote efficient targeted integration without selection pressure in a human cell line.

### Example 9

### Application: GOI Targeted Integrations in DMD Gene of Different Human Cell Lines Using Specific Meganucleases

### Example 9.1

### Transfection and Selection

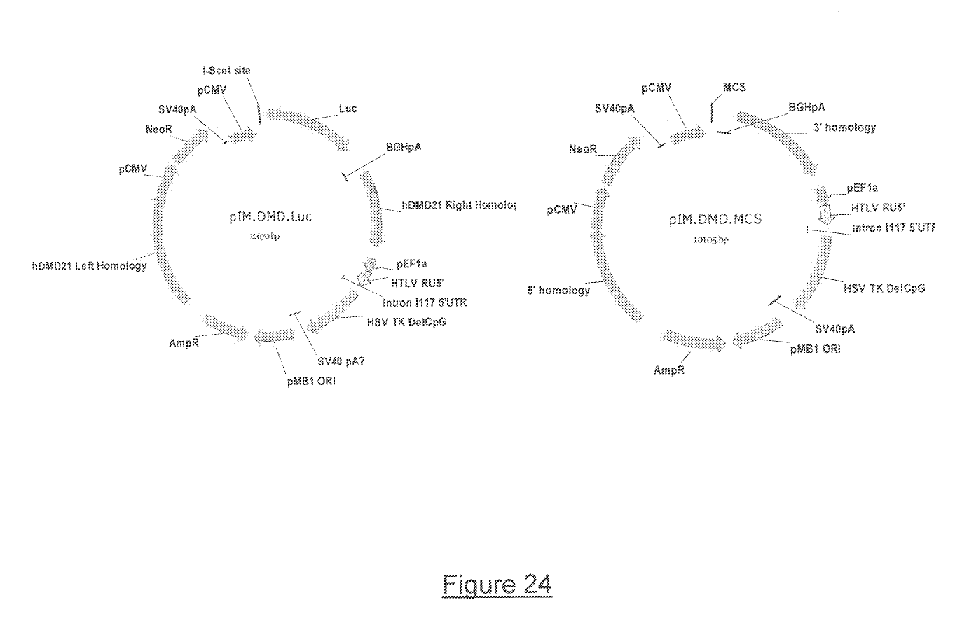

In this example, the technical process leading to the identification of gene of interest (GOI) targeted integration using a meganuclease specific for a target located in the DMD human gene is presented. Plasmid maps related to DMD-specific integration matrices that have been used for the demonstrations given here below [pIM-DMD-MCS (SEQ ID NO: 112) pIM-DMD-Luc (SEQ ID NO: 113)] are depicted in FIG. 24. Since the engineered meganuclease can recognize and cut within the human DMD gene, targeted integration can be obtained in virtually all human cell lines. Depending of the capacity of cells to adhere to plastic, transfection and selection procedures are different but both lead to the efficient identification of targeted clones.

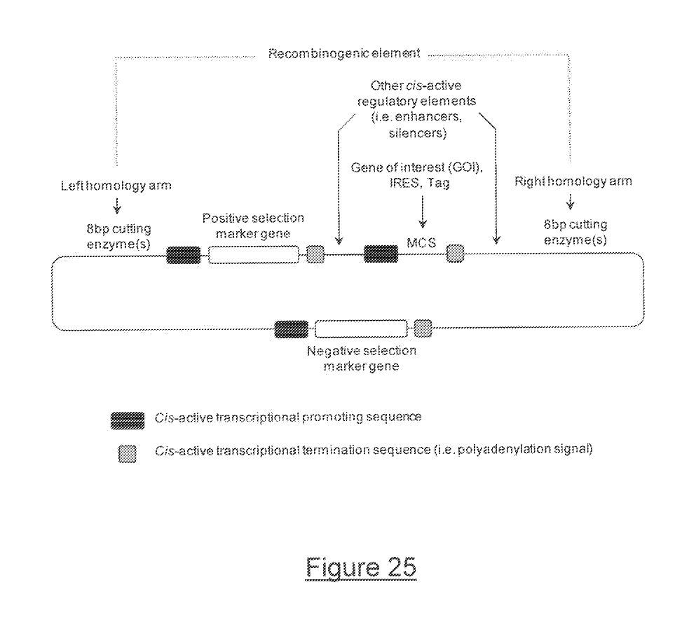

Universal plasmid backbones have been designed and constructed in order to allow meganuclease driven homologous recombination in any cell type (FIG. 25). Certain genetic elements which are cloned in the integration matrix are mandatory such as the homology arms, the selection cassette and the GOI expression cassette. The homology arms are necessary to achieve specific gene targeting. They are produced by PCR amplification using specific primers for i) the genomic region upstream of the meganuclease target site (left homology arm) and ii) the genomic region downstream of the meganuclease target site (right homology arm). The positive selection cassette is composed of a resistance gene controlled by a promoter region and a terminator sequence, which is also the case for the counter (negative) selection cassette. In pIM-DMD plasmids (FIG. 24, SEQ ID NO: 112 and 113), positive and negative selection marker genes are respectively neomycin and HSV TK. The expression cassette is composed of a multiple cloning site (MCS) where the GOI is cloned using classical molecular biology techniques. The MCS is flanked by promoter (upstream) and terminator (downstream) sequences. In pIM-DMD plasmids, the promoter is pCMV and the terminator sequence is bovine growth hormone polyadenylation signal BGH pA as described in FIG. 24.

Integration matrix and meganuclease expression vector are transfected into cells using known techniques. There are various methods of introducing foreign DNA into a eukaryotic cell and many materials have been used as carriers for transfection, which can be divided into three kinds: (cationic) polymers, liposomes and nanoparticles. Other methods of transfection include nucleofection, electroporation, heat shock, magnetofection and proprietary transfection reagents such as Lipofectamine, Dojindo Hilymax, Fugene, JetPEI, Effectene, DreamFect, PolyFect, Nucleofector, Lyovec, Attractene, Transfast, Optifect.

a) Transfection and Selection of Adherent HEK-293 Cells

As an example, the procedure used for the transfection of HEK-293 (human adherent cell line) with Lipofectamine® is described below.

### Materials and Methods

One day prior to transfection, HEK-293 cells were seeded in a 10 cm tissue culture dish (10⁶ cells per dish). On transfection day (D), Human DMD meganuclease expression plasmid and integration matrix (pIM-DMD-MCS and its derived GOI-containing plasmid with the GOI in place of the MCS, or pIM-DMD-Luc as positive control) were diluted in 300 μl of serum-free medium. On the other hand, 10 μl of Lipofectamine® reagent was diluted in 290 μl of serum-free medium. Both mixes were incubated 5 minutes at room temperature. Then, the diluted DNA was added to the diluted Lipofectamine® reagent (and never the way around). The mix was gently homogenized by tube inversion and was incubated 20 minutes at room temperature. The transfection mix was then dispensed over plated cells and transfected cells were incubated in a 37° C., 5% CO₂ humidified incubator. The next day, transfection medium was replaced with fresh complete medium.

Three days after transfection, cells were harvested and counted. Cells were then seeded in 10 cm tissue culture dishes at the density of 200 cells/ml in a total volume of 10 ml of complete medium. 10 cm tissue culture dishes were incubated at 37° C., 5% CO₂ for a total period of 7 days. At the end of the 7 days period, single colonies of cells were visible.

Ten days after transfection (or seven days after plating), culture medium was replaced with fresh medium supplemented with selection agent (i.e. corresponding to the resistance gene present on the integration matrix). In this example, the integration matrix contains a full neomycin resistance gene. Therefore, selection of clones was done with G418 sulfate at the concentration of 0.4 mg/ml. The medium replacement was done every two or three days for a total period of seven days. At the end of this selection phase, resistant cells were either isolated in a 96-well plates or were maintained in the 10 cm dish (adherent cells) or re-arrayed in new 96-well plates (suspension cells) for counter selection.

Since the HSV TK counter selection marker is present on the integration matrix, resistant cells or colonies can be cultivated in the presence of 10 μM of ganciclovir (GCV) to eliminate unwanted integration events such as random integration. After 5 days of culture in the presence of GCV, double resistant (G418^{R}-GCV^{R}) cell colonies can be isolated for further characterization.

At the end of this selection phase, resistant (G418^{R}-GCV^{R}) cell colonies can be isolated for molecular screening by PCR (see example 9.2 below).

b) Transfection and Selection of Adherent U-2 OS Cells

As another example, the procedure used for the transfection of U-2 OS (human adherent cell line) with the Amaxa® Cell Line Nucleofector® Kit V reagents commercialized by Lonza is described below.

Materials and Methods

On transfection day (D), cells were not more than 80% confluent. Cells were harvested from their sub-culturing vessel (T162 Tissue Culture Flask) by trypsinization and were collected in a 15 ml conical tube. Harvested cells were counted. 10⁶ cells were needed per transfection point. Cells were centrifuged at 300 g for 5 min and were resuspended in Cell Line Nucleofector® Solution V at the concentration of 10⁶ cells/100 μl. Amaxa electroporation cuvette was prepared by adding i) the integration matrix (pIM-DMD-MCS and its derived GOI-containing plasmid with the GOI in place of the MCS, or pIM-DMD-Luc as positive control) and the hsDMD Meganuclease Plasmid ((Endofree quality preparation), ii) 100 μl of cell suspension (10⁶ cells). Cells and DNA were gently mixed and electroporated using Amaxa® program X-001. Immediately after electroporation, pre-warmed complete medium was added to cells and cells suspension was split into two 10 cm dishes (5 ml per dish) containing 5 ml of 37° C. pre-warmed complete medium. 10 cm dishes were then incubated in a 37° C., 5% CO₂ humidified incubator.

Two days after transfection (D+2) the complete culture medium was replaced with fresh complete medium supplemented with 0.4 mg/ml of G418. This step was repeated every 2 or 3 days for a total period of 7 days. At D+9, the complete culture medium supplemented with 0.4 mg/ml G418 was replaced with fresh complete medium supplemented with 0.4 mg/ml of G418 and 50 μM Ganciclovir. This step was repeated every 2 or 3 days for a total period of 5 days. At D+14, G418 and GCV resistant clones were picked in a 96-well plate. At this step cells were maintained in complete medium supplemented with 0.4 mg/ml of G418 only.

At the end of this selection phase, resistant (G418^{R}-GCV^{R}) cell colonies were isolated for molecular screening by PCR (see example 9.2 below).

Transfection and Selection of Adherent HCT116 Cells

As another example, the procedure used for the transfection of HCT 116 (human adherent cell line) with FuGENE® HD is described below.

Materials and Methods

One day prior to transfection, HCT 116 cells were seeded in a 10 cm tissue culture dish (5×10⁵ cells per dish). On transfection day (D), Human DMD meganuclease expression plasmid and integration matrix (pIM-DMD-MCS and its derived GOI-containing plasmid with the GOI in place of the MCS, or pIM-DMD-Luc as positive control) were diluted in 500 μl of serum-free medium. Then, 15 μl of FuGENE® HD reagent was diluted in the DNA mix. The mix was gently homogenized by tube inversion and incubated 15 minutes at room temperature. The transfection mix was then dispensed over plated cells and transfected cells were incubated in a 37° C., 5% CO₂ humidified incubator.

The day after transfection (D+1), the complete culture medium was replaced with fresh complete medium supplemented with 0.4 mg/ml of G418. This step was repeated every 2 or 3 days for a total period of 7 days. At D+9, the complete culture medium supplemented with 0.4 mg/ml G418 was replaced with fresh complete medium supplemented with 0.4 mg/ml of G418 and 50 μM Ganciclovir. This step was repeated every 2 or 3 days for a total period of 5 days. At D+14, G418 and GCV resistant clones were picked in a 96-well plate. At this step, cells were maintained in complete medium supplemented with 0.4 mg/ml of G418 only.

At the end of this selection phase, resistant (G418^{R}-GCV^{R}) cell colonies were isolated for molecular screening by PCR (see example 9.2 below).

### Example 9.2

### PCR Screening Once the selection and optionally counter selection was achieved, resistant colonies or clones were re-arrayed in 96-well plates and maintained in the 96-well format. Replicas of plates were done in order to generate genomic DNA from resistant cells. PCR were then performed to identify targeted integration.

Materials and Methods

Genomic DNA preparation: genomic DNAs (gDNAs) from double resistant cell clones were prepared with the ZR-96 Genomic DNA Kit™ (Zymo Research) according to the manufacturer's recommendations.

PCR Primer Design:

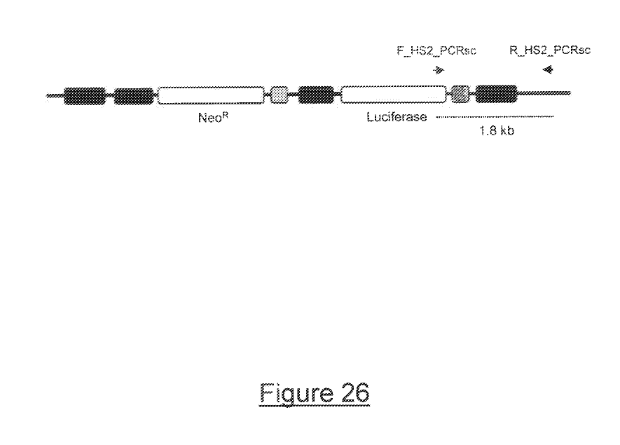

In the present example (human DMD locus), PCR primers were chosen according to the following rules and as represented in FIG. 26. The forward primer is located in the heterologous sequence (i.e. between the homology arms). For instance the forward PCR primer is situated in the BGH polyA sequence (F_HS2_PCR_{SC}: CCTTCCTTGACCCTGGAAGGTGCCACTCCC; SEQ ID NO: 114), terminating the transcription of the GOI. The reverse PCR primer is located within the DMD locus but outside the right homology arm (R_HS2_PCR_{SC}: TTAAACACTGCTATTCAGTAGGACACACACC; SEQ ID NO: 115). Therefore, PCR amplification was possible only when a specific targeted integration occurs. Moreover, this combination of primers can be used for the screening of targeted events, independently to the GOI to be integrated.

PCR Conditions:

PCR reactions were carried out on 5 μl of gDNA in 25 μl final volume with 0.25 μM of each primers, 10 μM of dNTP and 0.5 μl of Herculase II FusionDNA polymerase (Stratagene).

PCR program:

| Temperature | Time | Cycle |
|---|---|---|
| (° C.) | (minutes) | number |
| 95 | 2 | 1 |
| 95 | 0.5 | 30 |
| 68 | 0.5 | |
| 72 | 1.5 | |
| 72 | 5 | 1 |

### Results

According to this molecular screening by PCR, results of targeted integration into the hsDMD locus of the different human cell lines, for which a specific protocol has been developed (see §a) to c)) are summarized in Table IX. The level of specific targeted integration was comprised between 7% and 44%, demonstrating the efficacy of the cGPS custom system. It also demonstrate that the system could be applied to any kind of cell lines (adherent, suspension, primary cell lines), providing that an adapted protocol is optimized.

TABLE IX
Summary of targeted integration in the different cell lines.

| | | Targeted clones (%) | Single copy integrants (%) |
|---|---|---|---|
| Adherent | HEK-293 | 41 | 41.5 |
| cell line | U-2 OS | 14 | 54 |
| | HCT 116 | 4 | 31 |

In order to further characterize these positive clones, cells from corresponding wells were maintained in culture and individually amplified from the 96-well plate format to a 10 cm dish culture format.

### Example 9.3

### Molecular Characterization (Southern Blot)

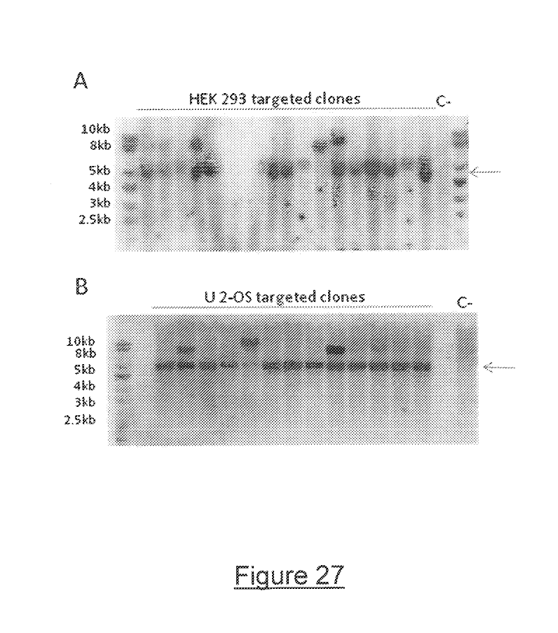

A correct targeted integration in double resistant clones can be easily identified at the molecular level by Southern blot analysis (FIG. 27).

Materials and Methods

gDNA from targeted clones was purified from 10⁷ cells (about a nearly confluent 10 cm dish) using the Blood and Cell culture DNA midi kit (Qiagen). 5 to 10 μg of gDNA was digested with a 10-fold excess of restriction enzyme by overnight incubation (here EcoRV restriction enzymes). Digested gDNA was separated on a 0.8% agarose gel and transfer on nylon membrane. Nylon membranes were then probed with a ³²P DNA probe specific for the neomycin gene. After appropriate washes, the specific hybridization of the probe was revealed by autoradiography (panel A: HEK-293 targeted clones; panel B: U 2-OS targeted clones).

Results

In the example presented here, the hybridization pattern of 15 HEK 293 targeted clones (panel A: FIG. 27) and 13 U 2-OS targeted clones (panel B: FIG. 27) were analyzed. According to the chosen restriction enzyme and the specific neo probe, a band at 4.8 kb in targeted clones was expected in contrast to the negative control (native untargeted cell line). It has been shown that the 4.8 kb band was present in 14 out of 15 HEK293 clones while this band was present in all U 2-OS targeted clones. These results demonstrate the efficiency of the DMD meganuclease driven targeted integration of exogenous DNA sequence, provided an efficient transfection and selection process.

### Example 10

### Luciferase Expression

In this example, the level of expression of luciferase under the control of 6 different promoters in HEK293 targeted clones was monitored.

The firefly luciferase reporter gene was cloned in different pIM-DMD-MCS vectors. The resulting vectors were transfected in HEK293 cells according to the protocol described in example 9.1, section a). Targeted cell clones surviving the selection and counter selection processes as described in example 9.1, section a) are isolated and characterized according to examples 9.2 and 9.3.

Materials and Methods

Luciferase Expression:

Cells from targeted clones were washed twice in PBS then incubated with 5 ml of trypsin-EDTA solution. After 5 min. incubation at 37° C., cells were collected in a 15 ml conical tube and counted.

Cells were then resuspended in complete DMEM medium at the density of 50,000 cells/ml. 100 μl (5,000 cells) were aliquoted in triplicate in a white 96-well plate (Perkin-Elmer). 100 μl of One-Glo reagent (Promega) was added per well and after a short incubation the plate was read on a microplate luminometer (Viktor, Perkin-Elmer).

Results

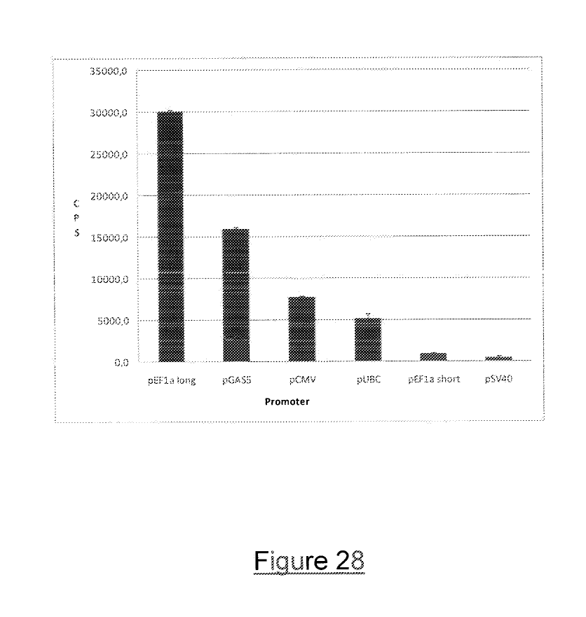

Corresponding data are presented in FIG. 28. For each promoter, the mean level of luciferase expression for 3 clones is shown. These data indicates that expression of the luciferase reporter gene is directly dependant on the strength of the chosen promoter, allowing the modulation of expression of a gene of interest.

### LIST OF REFERENCES CITED IN THE DESCRIPTION

1. Ohshima S, Shin J-H, Yuasa K, Nishiyama A, Kira J, Okada T et al. The journal of the American Society of Gene Therapy 2009; 17: 73-80.
2. Liu M, Yue Y, Harper S Q, Grange R W, Chamberlain J S, Duan D. Mol Ther 2005; 11: 245-256.
3. Lai Y, Yue Y, Liu M, Duan D. Hum Gene Ther 2006; 17: 1036-1042.
4. Wang Z, Kuhr C S, Allen J M, Blankinship M, Gregorevic P, Chamberlain J S et al. Mol Ther 2007; 15: 1160-1166.
5. Odom G L, Gregorevic P, Allen J M, Finn E, Chamberlain J S. Mol Ther 2008; 16: 1539-1545.
6. Harper S Q, Hauser M A, DelloRusso C, Duan D, Crawford R W, Phelps S F et al. Nat Med 2002; 8: 253-261.
7. Peault B, Rudnicki M, Torrente Y, Cossu G, Tremblay J P, Partridge T et al. Mol Ther 2007; 15: 867-877.
8. Deasy B M, Jankowski R J, Huard J. Blood Cells Mol Dis 2001; 27: 924-933.
9. Ikemoto M, Fukada S-I, Uezumi A, Masuda S, Miyoshi H, Yamamoto H et al. Molecular therapy: the journal of the American Society of Gene Therapy 2007; 15: 2178-2185.
10. Sampaolesi M, Blot S, D'Antona G, Granger N, Tonlorenzi R, Innocenzi A et al. Nature 2006; 444: 574-579.
11. Skuk D, Goulet M, Roy B, Chapdelaine P, Bouchard J-P, Roy R et al. Journal of neuropathology and experimental neurology 2006; 65: 371-386.
12. Skuk D, Goulet M, Roy B, Piette V, Cote C H, Chapdelaine P et al. Neuromuscul Disord 2007; 17: 38-46.
13. Welch E M, Barton E R, Zhuo J, Tomizawa Y, Friesen W J, Trifillis P et al. Nature 2007; 447: 87-91.
14. Wilton S. PTC124, Neuromuscul Disord 2007; 17: 719-720.
15. Williams J H, Schray R C, Sirsi S R, Lutz G J. BMC Biotechnol 2008; 8: 35.
16. Jearawiriyapaisarn N, Moulton H M, Buckley B, Roberts J, Sazani P, Fucharoen S et al. Mol Ther 2008; 16: 1624-1629.
17. Yokota T, Duddy W, Partridge T. Acta Myol 2007; 26: 179-184.
18. Gaspar, H. B. et al. Lancet 364, 2181-7 (2004).
19. Cavazzana-Calvo, M. et al. Science 288, 669-72 (2000).
20. Aiuti, A. et al. Science 296, 2410-3 (2002).
21. De Luca, M., Pellegrini, G. & Mavilio, F. Br J Dermatol (2009).
22. Bainbridge, J. W. et al. N Engl J Med 358, 2231-9 (2008).
23. Maguire, A. M. et al. N Engl J Med 358, 2240-8 (2008).
24. Hacein-Bey-Abina, S. et al. J Clin Invest 118, 3132-42 (2008).
25. Hacein-Bey-Abina, S. et al. Science 302, 415-9 (2003).
26. Howe, S. J. et al. J Clin Invest 118, 3143-50 (2008).
27. Chang, A. H. & Sadelain, M. Mol Ther 15, 445-56 (2007).
28. May, C. et al. Nature 406, 82-6 (2000).
29. Sadelain, M. Curr Opin Hematol 13, 142-8 (2006).
30. Ellis, J. Hum Gene Ther 16, 1241-6 (2005).
31. Yu, S. F. et al. Proc Natl Acad Sci USA 83, 3194-8 (1986).
32. Yee, J. K. et al. Proc Natl Acad Sci USA 84, 5197-201 (1987).
33. Puchta et al. Nucleic Acids Res 1993 21: 5034-5040
34. Rouet et al. Mol Cell Biol 1994 14: 8096-8106
35. Choulika et al. Mol Cell Biol 1995 15: 1968-1973
36. Puchta et al. Proc Natl Acad Sci U.S.A 1996 93: 5055-5060
37. Sargent et al. Mol Cell Biol 1997 17: 267-277
38. Cohen-Tannoudji et al. Mol Cell Biol 1998 18: 1444-1448
39. Donoho et al. Mol Cell Biol 1998 18: 4070-4078
40. Elliott et al. Mol Cell Biol 1998 18: 93-101
41. Chevalier and Stoddard Nucleic Acids Res 2001 29: 3757-3774
42. Smith et al. Nucleic Acids Res 1999 27: 674-681
43. Bibikova et al. Mol Cell Biol 2001 21: 289-297
44. Bibikova et al. Genetics 2002 161: 1169-1175
45. Bibikova et al. Science 2003 300: 764
46. Porteus and Baltimore Science 2003 300: 763
47. Alwin et al. Mol Ther 2005 12: 610-617
48. Urnov et al. Nature 2005 435: 646-651
49. Porteus M. H. Mol Ther 2006 13: 438-446
50. Pabo et al. Annu Rev Biochem 2001 70: 313-340
51. Jamieson et al. Nat Rev Drug Discov 2003 2: 361-368
52. Rebar and Pabo Science 1994 263: 671-673
53. Kim and Pabo Proc Natl Acad Sci USA 1998 95: 2812-2817
54. Klug et al. Proc Natl Acad Sci USA 1994 91: 11163-11167
55. Isalan and Klug Nat Biotechnol 2001 19: 656-660
56. Catto et al. Nucleic Acids Res 2006 34: 1711-1720
57. Chevalier et al. Nat Struct Biol 2001 8: 312-316
58. Chevalier et al. J Mol Biol 2003 329: 253-269
59. Moure et al. J Mol Biol 2003 334: 685-693,
60. Silva et al. J Mol Biol 1999 286: 1123-1136
61. Bolduc et al. Genes Dev 2003 17: 2875-2888
62. Ichiyanagi et al. J Mol Biol 2000 300: 889-901
63. Moure et al. Nat Struct Biol 2002 9: 764-770
64. Chevalier et al. Mol Cell 2002 10: 895-905
65. Epinat et al. Nucleic Acids Res 2003 31: 2952-62
66. Seligman et al. Genetics 1997 147: 1653-1664
67. Sussman et al. J Mol Biol 2004 342: 31-41
68. Arnould et al. J Mol Biol 2006 355: 443-458
69. Rosen et al. Nucleic Acids Res 2006 34: 4791-4800
70. Smith et al. Nucleic Acids Res 2006 34 e149
71. Doyon et al. J Am Chem Soc 2006 128: 2477-2484
72. Gimble et al. J Mol Biol 2003 334: 993-1008
73. Ashworth et al. Nature 2006 441: 656-659
74. Argast et al. J Mol Biol 1998 280: 345-353
75. Rosenecker, J., Huth, S. & Rudolph, C. Curr Opin Mol Ther 8, 439-45 (2006).

1. An I-CreI variant, comprising at least two I-CreI monomers, wherein at least one of the two I-CreI monomers comprises at least two substitutions, one in each of two functional subdomains of a LAGLIDADG core domain situated from positions 26 to 40 and 44 to 77 of I-CreI, the variant being able to cleave a DNA target sequence from a dystrophin gene (DMD), and wherein the I-CreI variant is obtained by a method comprising:
(a) constructing a first series of I-CreI variants comprising a substitution of at least one position selected from the group consisting of 26, 28, 30, 32, 33, 38 and 40 of a first functional subdomain of the LAGLIDADG core domain situated from positions 26 to 40 of I-CreI,
(b) constructing a second series of I-CreI variants comprising a substitution of at least one position selected from the group consisting of 44, 68, 70, 75 and 77 of a second functional subdomain of the LAGLIDADG core domain situated from positions 44 to 77 of I-CreI,
(c) selecting, screening, or selecting screening the variants from the first series of (a) which are able to cleave a mutant I-CreI site wherein
(i) a nucleotide triplet in positions −10 to −8 of the I-CreI site has been replaced with a nucleotide triplet which is present in positions −10 to −8 of the DNA target sequence from DMD and
(ii) a nucleotide triplet in positions +8 to +10 has been replaced with a reverse complementary sequence of a nucleotide triplet which is present in positions −10 to −8 of the DNA target sequence from DMD,
(d) selecting, screening, or selecting screening the variants from the second series of (b) which are able to cleave a mutant I-CreI site wherein
(i) a nucleotide triplet in positions −5 to −3 of the I-CreI site has been replaced with a nucleotide triplet which is present in positions −5 to −3 of the DNA target sequence from DMD and
(ii) a nucleotide triplet in positions +3 to +5 has been replaced with a reverse complementary sequence of the nucleotide triplet which is present in positions −5 to −3 of the DNA target sequence from DMD,
(e) selecting, screening, or selecting screening the variants from the first series of (a) which are able to cleave a mutant I-CreI site wherein
(i) a nucleotide triplet in positions +8 to +10 of the I-CreI site has been replaced with a nucleotide triplet which is present in positions +8 to +10 of the DNA target sequence from DMD and
(ii) a nucleotide triplet in positions −10 to −8 has been replaced with a reverse complementary sequence of the nucleotide triplet which is present in positions +8 to +10 of the DNA target sequence from DMD,
(f) selecting, screening, or selecting screening the variants from the second series of (b) which are able to cleave a mutant I-CreI site wherein
(i) a nucleotide triplet in positions +3 to +5 of the I-CreI site has been replaced with a nucleotide triplet which is present in positions +3 to +5 of the DNA target sequence from DMD and
(ii) a nucleotide triplet in positions −5 to −3 has been replaced with a reverse complementary sequence of the nucleotide triplet which is present in positions +3 to +5 of the DNA target sequence from DMD, and
wherein the method further comprises (g), (h), or (g) and (h) comprising:
(g) combining in a single variant, the mutation or mutations in positions 26 to 40 and 44 to 77 of two variants from (c) and (d), to obtain a novel homodimeric I-CreI variant which cleaves a sequence wherein
(i) the nucleotide triplet in positions −10 to −8 is identical to the nucleotide triplet which is present in positions −10 to −8 of the DNA target sequence from DMD,
(ii) the nucleotide triplet in positions +8 to +10 is identical to the reverse complementary sequence of the nucleotide triplet which is present in positions −10 to −8 of the DNA target sequence from DMD,
(iii) the nucleotide triplet in positions −5 to −3 is identical to the nucleotide triplet which is present in positions −5 to −3 of the DNA target sequence from DMD and
(iv) the nucleotide triplet in positions +3 to +5 is identical to the reverse complementary sequence of the nucleotide triplet which is present in positions −5 to −3 of the DNA target sequence from DMD, and
(h) combining in a single variant, the mutation or mutations in positions 26 to 40 and 44 to 77 of two variants from (e) and (f), to obtain a novel homodimeric I-CreI variant which cleaves a sequence wherein
(i) the nucleotide triplet in positions +8 to +10 of the I-CreI site has been replaced with the nucleotide triplet which is present in positions +8 to +10 of the DNA target sequence from DMD,
(ii) the nucleotide triplet in positions −10 to −8 is identical to the reverse complementary sequence of the nucleotide triplet in positions +8 to +10 of the DNA target sequence from DMD,
(iii) the nucleotide triplet in positions +3 to +5 is identical to the nucleotide triplet which is present in positions +3 to +5 of the DNA target sequence from DMD,
(iv) the nucleotide triplet in positions −5 to −3 is identical to the reverse complementary sequence of the nucleotide triplet which is present in positions +3 to +5 of the DNA target sequence from DMD, and
wherein the method further comprises:
(i) combining at least one variant obtained in (g) or (h) to form a heterodimer, and
(j) selecting, screening, or selecting and screening the heterodimer from (i) which is able to cleave the DNA target sequence from DMD.

2. (canceled)

3. (canceled)

4. (canceled)

5. (canceled)

6. (canceled)

7. The variant of claim 1, which comprises a substitution in positions 137 to 143 of I-CreI that modifies the specificity of the variant towards the nucleotide in at least one position selected from the group consisting of positions +1 to 2, +6 to 7 and +11 to 12 of the target site in DMD.

8. The variant of claim 1, which comprises a substitution on the entire I-CreI sequence that improves binding, cleavage, or binding cleavage properties of the variant towards the DNA target sequence from DMD.

9. The variant of claim 1, wherein the substitutions are replacements of the initial amino acids wherein the amino acids are selected from the group consisting of A, D, E, F, G, H, I, K, M, N, P, Q, R, S, T, Y, C, W, L and V.

10. The variant of claim 1, wherein the variant is a heterodimer, resulting from the association of a first and a second monomer comprising different mutations in positions 26 to 40 and 44 to 77 of I-CreI, wherein the heterodimer is able to cleave a non-palindromic DNA target sequence from DMD.

11. The variant of claim 10, wherein the variant is an obligate heterodimer, wherein the first and the second monomer, respectively, further comprises a D137R mutation and a R51D mutation.

12. The variant of claim 10, wherein the variant is an obligate heterodimer, wherein
the first monomer further comprises at least one mutation selected from the group consisting of K7R, E8R, E61R, K96R and L97F mutations or at least one mutation selected from the group consisting of K7R, E8R, F54W, E61R, K96R and L97F mutations, and
the second monomer further comprises at least one mutation selected from the group consisting of K7E, F54G, L58M and K96E mutations or at least one mutation selected from the group consisting of K7E, F54G, K57M and K96E mutations.

13. The variant according to claim 1, wherein the variant comprises a single polypeptide chain comprising two monomers or core domains of one or two variants.

14. The variant of claim 13, wherein the variant comprises the first and the second monomers connected by a peptide linker.

15. (canceled)

16. The variant according to claim 14, wherein the variant is selected from the group consisting of SEQ ID NO: 62 to SEQ ID NO: 105, SEQ ID NO: 116 to SEQ ID NO: 119, SEQ ID NO: 121 and SEQ ID NO: 122 to SEQ ID NO: 130.

17. A polynucleotide fragment encoding the variant of claim 1.

18. An expression vector comprising the polynucleotide fragment of claim 17.

19. The expression vector according to claim 18, further comprising a transgene and two sequences homologous to the genomic sequence flanking the target sequence by the variant from DMD.

20. A host cell which comprises the polynucleotide of claim 17.

21. Currently amended): A host cell which comprises the vector of claim 18.

22. A non-human transgenic animal which comprises the polynucleotide of claim 17.

23. A non-human transgenic animal which comprises the vector of claim 18.

24. A transgenic plant which comprises the polynucleotide of claim 17.

25. A transgenic plant which comprises the vector of claim 18.

26. A pharmaceutical composition comprising the variant of claim 1 and a pharmaceutically active carrier.

27. A pharmaceutical composition comprising the expression vector of claim 18.

28. A pharmaceutical composition comprising the expression vector of claim 19.

29. A method of treatment of a genetic disease caused by a mutation in a DMD gene, wherein the method comprises administering to a subject in need thereof an effective amount of the variant of claim 1.

30. A method of treatment of a genetic disease caused by a mutation in a gene other than DMD, wherein the method comprises administering to a subject in need thereof an effective amount of the variant of claim 1.

31. A method for inserting a transgene into a genomic DMD locus of a cell, a tissue or a non-human animal, wherein the variant of claim 1 is introduced into the cell, the tissue or the non-human animal.
